(19) United States
(12) Patent Application Publication
(10) Pub. No.: US 2023/0340124 A1
(43) Pub. Date: Oct. 26, 2023

(54) METHODS OF TREATING AN INDIVIDUAL THAT HAS FAILED AN ANTI-PD-1/ANTI-PD-L1 THERAPY

(71) Applicant: President and Fellows of Harvard College, Cambridge, MA (US)

(72) Inventors: Gordon J. Freeman, Brookline, MA (US); Francesca S. Gazzaniga, Jamaica Plain, MA (US); Joon Seok Park, Brookline, MA (US); Arlene H. Sharpe, Brookline, MA (US); Dennis L. Kasper, Brookline, MA (US)

(21) Appl. No.: 18/026,746

(22) PCT Filed: Sep. 16, 2021
(86) PCT No.: PCT/US21/50674

§ 371 Date: Mar. 16, 2023

Related U.S. Application Data
(60) Provisional application No. 63/165,574, filed on Mar. 24, 2021.
(60) Provisional application No. 63/079,245, filed on Sep. 16, 2020.

(51) Int. Cl. C07K 16/28; C07K 16/22

(52) U.S. Cl. CPC C07K 16/2818; C07K 16/2827; C07K 16/22; A61K 2039/507

(57) ABSTRACT

Provided herein are methods of treating cancer in an individual that has failed an anti-PD1/PD-L1 therapy, comprising selecting an individual that has failed a prior anti-PD1/PD-L1 therapy; and administering to the individual a first agent that blocks or disrupts PD-L2, RGMb, or a combination thereof, and a second agent that blocks or disrupts PD-L1, PD-1 or a combination thereof. Also provided herein are kits and therapeutic compositions for use in the methods described herein.

### RELATED APPLICATIONS

This application claims priority to U.S. Provisional Application 63/079,245, filed Sep. 16, 2020, and U.S. Provisional Application 63/165,574, filed Mar. 24, 2021, each of which is incorporated herein by reference in its entirety.

### STATEMENT AS TO FEDERALLY SPONSORED RESEARCH

This invention was made with government support under Grant No. 5T32HD55148-10 awarded by the National Institutes of Health. The government has certain rights in the invention.

### SUMMARY

Described herein, in some embodiments, is a method for treating cancer in an individual that has failed an immune checkpoint inhibitor therapy, comprising: a) selecting an individual that has failed a prior immune checkpoint inhibitor therapy, wherein the prior immune checkpoint inhibitor therapy comprises administering to the individual a therapeutic which blocks or disrupts an immune checkpoint protein; and b) administering to the individual i) a first agent that blocks or disrupts PD-L2, RGMb, or a combination thereof, and ii) a second agent that blocks or disrupts the immune checkpoint protein. In some embodiments, the immune checkpoint protein is selected from the group consisting of CTLA-4, PD-1, VISTA, B7-H2, B7-H3, PD-L1, B7-H4, B7-H6, ICOS, HVEM, CD160, gp49B, PIR-B, a KIR family receptor, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, or a combination thereof. Described herein, in some embodiments, is a method for treating cancer in an individual that has failed an anti-PD1/PD-L1 therapy, comprising: a) selecting an individual that has failed a prior anti-PD1/PD-L1 therapy; and b) administering to the individual i) a first agent that blocks or disrupts PD-L2, RGMb, or a combination thereof, and ii) a second agent that blocks or disrupts PD-L1, PD-1 or a combination thereof. In some embodiments, the first agent is an antibody, a non-activating form of PD-L2 or RGMb, a fusion protein, a nucleic acid molecule that blocks PD-L2 or RGMb transcription or translation, a small molecule, or a polypeptide. In some embodiments, the first agent is AMP-224 or CA-170. In some embodiments, the first agent is an antibody. In some embodiments, the first agent is an antibody that blocks or disrupts PD-L2. In some embodiments, the antibody that blocks or disrupts PD-L2 is a monoclonal antibody, or an antigen binding fragment thereof. In some embodiments, the antibody that blocks or disrupts PD-L2 binds the peptide sequence CFTVTVPKDLYVVEYGSN or CYRSMISYGGADYKRITV. In some embodiments, the antibody that blocks or disrupts PD-L2 comprises the heavy chain variable domain amino acid sequence encoded by SEQ ID NO: 3 and the light chain variable domain amino acid sequence encoded by SEQ ID NO: 5. In some embodiments, the antibody that blocks or disrupts PD-L2 comprises the heavy chain variable domain amino acid sequence encoded by SEQ ID NO: 4 and the light chain variable domain amino acid sequence encoded by SEQ ID NO: 6. In some embodiments, the antibody that blocks or disrupts PD-L2 is a humanized or fully human antibody. In some embodiments, the antibody that blocks or disrupts PD-L2 is a human anti-PD-L2 antibody that is structurally related to mouse anti-human PD-L2 antibodies 24F.10C12, GF17.2C9, MIH37, 3.2, or TY25. In some embodiments, the antibody that blocks or disrupts PD-L2 comprises a heavy chain variable region sequence comprising SEQ ID NOS:7-11 and/or a light chain variable region sequence comprising SEQ ID NO:12-14. In some embodiments, the antibody that blocks or disrupts PD-L2 is a bispecific antibody. In some embodiments, the first agent is an antibody that disrupts or blocks RGMb. In some embodiments, the antibody that disrupts or blocks RGMb is a monoclonal antibody. In some embodiments, the antibody that blocks or disrupts RGMb is a humanized antibody. In some embodiments, the antibody that disrupts or blocks RGMb comprises the heavy chain variable domain amino acid sequence encoded by SEQ ID NO: 17 and the light chain variable domain amino acid sequence encoded by SEQ ID NO: 16. In some embodiments, the antibody that blocks or disrupts RGMb is a bispecific antibody. In some embodiments, the second agent is an antibody. In some embodiments, the second agent is an antibody, a non-activating form of PD-L1, a fusion protein, a nucleic acid molecule that blocks PD-L1 transcription or translation, or a small molecule PD-L1 antagonist. In some embodiments, the second agent is an antibody that blocks PD-1. In some embodiments, the antibody that blocks PD-1 is a monoclonal antibody. In some embodiments, the antibody that blocks PD-1 is a humanized antibody. In some embodiments, the antibody that blocks PD-1 is a bispecific antibody. In some embodiments, the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188. In some embodiments, the second agent is an antibody that blocks PD-L1. In some embodiments, the antibody that blocks PD-L1 is a monoclonal antibody. In some embodiments, the antibody that blocks PD-L1 is a humanized antibody. In some embodiments, the antibody that blocks PD-L1 is a bispecific antibody. In some embodiments, the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244. In some embodiments, the first agent is administered to the subject systemically. In some embodiments, the first agent is administered orally. In some embodiments, the first agent is administered parenterally. In some embodiments, the first agent is administered intravenously. In some embodiments, the second agent is administered to the subject systemically. In some embodiments, the second agent is administered orally. In some embodiments, the second agent is administered parenterally. In some embodiments, the second agent is administered intravenously. In some embodiments, the cancer is a head and neck cancer lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, an ovarian cancer, a testicular cancer, a prostate cancer, a cervical cancer, a vaginal cancer, or a bladder cancer. In some embodiments, the cancer comprises a tumor. In some embodiments, the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

Described herein, in certain embodiments, is a therapeutic composition for treating an individual with cancer comprising, comprising: a) a first agent that blocks or disrupts PD-L2, RGMb, or a combination thereof, and b) a second agent that blocks or disrupts PD-L1, PD-1 or a combination thereof. In some embodiments, the therapeutic composition is for use in treating an individual that has failed an anti-PD1/PD-L1 therapy. In some embodiments, the first agent is an antibody, a non-activating form of PD-L2 or RGMb, a fusion protein, a nucleic acid molecule that blocks PD-L2 or RGMb transcription or translation, a small molecule, or a polypeptide. In some embodiments, the first agent is AMP-224, CA-170, or a combination thereof. In some embodiments, the first agent is an antibody. In some embodiments, the first agent is an antibody that blocks or disrupts PD-L2. In some embodiments, the antibody that blocks or disrupts PD-L2 is a monoclonal antibody, or an antigen binding fragment thereof. In some embodiments, the antibody that blocks or disrupts PD-L2 binds the peptide sequence CFTVTVPKDLYVVEYGSN or CYRSMISYGGADYKRITV. In some embodiments, the antibody that blocks or disrupts PD-L2 comprises the heavy chain variable domain amino acid sequence encoded by SEQ ID NO: 3 and the light chain variable domain amino acid sequence encoded by SEQ ID NO: 5. In some embodiments, the antibody that blocks or disrupts PD-L2 comprises the heavy chain variable domain amino acid sequence encoded by SEQ ID NO: 4 and the light chain variable domain amino acid sequence encoded by SEQ ID NO: 6. In some embodiments, the antibody that blocks or disrupts PD-L2 is a humanized antibody. In some embodiments, the antibody that blocks or disrupts PD-L2 is a human anti-PD-L2 antibody that is structurally related to mouse anti-human PD-L2 antibodies 24F.10C12, GF17.2C9, MIH37, 3.2, or TY25. In some embodiments, the antibody that blocks or disrupts PD-L2 comprises a heavy chain variable region sequence comprising SEQ ID NOS:7-11 and/or a light chain variable region sequence comprising SEQ ID NO:12-14. In some embodiments, the antibody that blocks or disrupts PD-L2 is a bispecific antibody. In some embodiments, the first agent is an antibody that disrupts or blocks RGMb. In some embodiments, the antibody that disrupts or blocks RGMb is a monoclonal antibody. In some embodiments, the antibody that blocks or disrupts RGMb is a humanized antibody. In some embodiments, the antibody that disrupts RGMb, comprises the heavy chain variable domain amino acid sequence encoded by SEQ ID NO: 17 and the light chain variable domain amino acid sequence encoded by SEQ ID NO: 16. In some embodiments, the antibody that disrupts RGMb, wherein the antibody that blocks or disrupts RGMb is a bispecific antibody. In some embodiments, the second agent is an antibody. In some embodiments, the second agent is an antibody, a non-activating form of PD-L1, a nucleic acid molecule that blocks PD-L1 transcription or translation, or a small molecule PD-L1 antagonist. In some embodiments, the second agent is an antibody that blocks PD-1. In some embodiments, the antibody that blocks PD-1 is a monoclonal antibody. In some embodiments, the antibody that blocks PD-1 is a humanized antibody. In some embodiments, the antibody that blocks PD-1 is a bispecific antibody. In some embodiments, the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188. In some embodiments, the second agent is an antibody that blocks PD-L1. In some embodiments, the antibody that blocks PD-L1 is a monoclonal antibody. In some embodiments, the antibody that blocks PD-L1 is a humanized antibody. In some embodiments, the antibody that blocks PD-L1 is a bispecific antibody. In some embodiments, the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244. In some embodiments, composition is administered to the subject systemically. In some embodiments, the composition is administered orally. In some embodiments, the composition is administered parenterally. In some embodiments, the composition is administered intravenously. In some embodiments, the cancer is a head and neck cancer, lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, an ovarian cancer, a testicular cancer, a prostate cancer, a cervical cancer, a vaginal cancer, or a bladder cancer. In some examples, the head and neck cancer is a squamous cell carcinoma, a lymphoma, an adenocarcinoma, or a sarcoma. In some embodiments, the head and neck cancer is a head and neck squamous cell carcinoma. In some embodiments, the cancer comprises a tumor. In some embodiments, the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

Described herein, in certain embodiments, is a kit comprising: a) a first agent that blocks or disrupts PD-L2, RGMb, or a combination thereof; b) a second agent that disrupts PD-L1, PD-1 or a combination thereof; and c) instructions for use of the first agent and the second agent in treating a cancer in an individual. In some embodiments, the first agent is an antibody, a non-activating form of PD-L2 or RGMb, a fusion protein, a nucleic acid molecule that blocks PD-L2 or RGMb transcription or translation, a small molecule, or a polypeptide. In some embodiments, the first agent is AMP-224, CA-170, or a combination thereof. In some embodiments, the first agent is an antibody. In some embodiments, the first agent is an antibody that blocks or disrupts PD-L2. In some embodiments, the antibody that blocks or disrupts PD-L2 is a monoclonal antibody, or an antigen binding fragment thereof. In some embodiments, antibody that blocks or disrupts PD-L2 binds the peptide sequence CFTVTVPKDLYVVEYGSN or CYRSMISYGGADYKRITV. In some embodiments, the antibody that blocks or disrupts PD-L2 comprises the heavy chain variable domain amino acid sequence encoded by SEQ ID NO: 3 and the light chain variable domain amino acid sequence encoded by SEQ ID NO: 5. In some embodiments, the antibody that blocks or disrupts PD-L2 comprises the heavy chain variable domain amino acid sequence encoded by SEQ ID NO: 4 and the light chain variable domain amino acid sequence encoded by SEQ ID NO: 6. In some embodiments, the antibody that blocks or disrupts PD-L2 is a humanized antibody. In some embodiments, the antibody that blocks or disrupts PD-L2 is a human anti-PD-L2 antibody that is structurally related to mouse anti-human PD-L2 antibodies 24F.10C12, GF17.2C9, MIH37, 3.2, or TY25. In some embodiments, the antibody that blocks or disrupts PD-L2 comprises a heavy chain variable region sequence comprising SEQ ID NOS:7-11 and/or a light chain variable region sequence comprising SEQ ID NOS:12-15. In some embodiments, the antibody that blocks or disrupts PD-L2 comprises a heavy chain variable region sequence of SEQ ID NO: 13 or 14, and a light chain variable region sequence of SEQ ID NO: 15, 16, or 17. In some embodiments, the antibody that blocks or disrupts PD-L2 is a bispecific antibody. In some embodiments, the first agent is an antibody that disrupts RGMb. In some embodiments, the antibody that disrupts RGMb is a monoclonal antibody. In some embodiments, the antibody that blocks or disrupts RGMb is a humanized antibody. In some embodiments, the antibody that disrupts RGMb is a human anti-RGMb antibody that is structurally related to 307.9D1, 307.8B2, 307.1H6, 307.9D3, or 307.5G1. In some embodiments, the antibody that disrupts or blocks RGMb, comprises the heavy chain variable domain amino acid sequence encoded by SEQ ID NO: 17 and the light chain variable domain amino acid sequence encoded by SEQ ID NO: 16. In some embodiments, the antibody that disrupts or blocks RGMb, wherein the antibody that blocks or disrupts RGMb is a bispecific antibody. In some embodiments, the second agent is an antibody, a non-activating form of PD-L1, a fusion protein, a nucleic acid molecule that blocks PD-L1 or transcription or translation, a small molecule, or a polypeptide. In some embodiments, the second agent is an antibody that blocks PD-1. In some embodiments, the antibody that blocks PD-1 is a monoclonal antibody. In some embodiments, the antibody that blocks PD-1 is a humanized antibody. In some embodiments, the antibody that blocks PD-1 is a bispecific antibody. In some embodiments, the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188. In some embodiments, the second agent is an antibody that blocks PD-L1. In some embodiments, the antibody that blocks PD-L1 is a monoclonal antibody. In some embodiments, the antibody that blocks PD-L1 is a humanized antibody. In some embodiments, the antibody that blocks PD-L1 is a bispecific antibody. In some embodiments, the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244. In some embodiments, the first agent is administered to the subject systemically. In some embodiments, the first agent is administered orally. In some embodiments, the first agent is administered parenterally. In some embodiments, the first agent is administered intravenously. In some embodiments, the second agent is administered to the subject systemically. In some embodiments, the second agent is administered orally. In some embodiments, the second agent is administered parenterally. In some embodiments, the second agent is administered intravenously. In some embodiments, the cancer is lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, an ovarian cancer, a testicular cancer, a prostate cancer, a cervical cancer, a vaginal cancer, or a bladder cancer. In some embodiments, the cancer comprises a tumor. In some embodiments, the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

Described herein, in certain embodiments, is a method for treating cancer in an individual that has failed a therapy selected from an anti-PD1 therapy or an anti-PD-L1 therapy, comprising administering to the individual i) a first agent that blocks or disrupts PD-L2, RGMb, or a combination thereof, and ii) a second agent that blocks or disrupts PD-L1, PD-1 or a combination thereof. In some embodiments, the cancer is refractory to an anti-PD1 therapy or an anti-PD-L1 therapy. In some embodiments, the cancer is not responsive to an anti-PD1 therapy or an anti-PD-L1 therapy. In some embodiments, the cancer has relapsed following an anti-PD1 therapy or an anti-PD-L1 therapy. In some embodiments, the patient has only been treated with the anti-PD1 therapy. In some embodiments, the patient has only been treated with the anti-PD-L1 therapy. In some embodiments, the patient has been treated with both the anti-PD1 therapy and the anti-PD-L1 therapy. In some embodiments, the anti-PD1 therapy is an antibody therapy. In some embodiments, the anti-PD-L1 therapy is selected from an antibody, a non-activating form of PD-L1, a fusion protein, a nucleic acid molecule that blocks PD-L1 transcription or translation, or a small molecule PD-L1 antagonist. In some embodiments, the anti-PD-L1 therapy is an antibody therapy. In some embodiments, the first agent is an antibody, a non-activating form of PD-L2 or RGMb, a fusion protein, a nucleic acid molecule that blocks PD-L2 or RGMb transcription or translation, a small molecule, or a polypeptide. In some embodiments, the first agent is AMP-224 or CA-170. In some embodiments, the first agent is an antibody. In some embodiments, the first agent is an antibody that blocks or disrupts PD-L2. In some embodiments, the antibody that blocks or disrupts PD-L2 is a monoclonal antibody, or an antigen binding fragment thereof. In some embodiments, the antibody that blocks or disrupts PD-L2 binds the peptide sequence CFTVTVPKDLYVVEYGSN or CYRSMISYGGADYKRITV. In some embodiments, the antibody that blocks or disrupts PD-L2 comprises the heavy chain variable domain amino acid sequence encoded by SEQ ID NO: 3 and the light chain variable domain amino acid sequence encoded by SEQ ID NO: 5. In some embodiments, the antibody that blocks or disrupts PD-L2 comprises the heavy chain variable domain amino acid sequence encoded by SEQ ID NO: 4 and the light chain variable domain amino acid sequence encoded by SEQ ID NO: 6. In some embodiments, the antibody that blocks or disrupts PD-L2 is a humanized or fully human antibody. In some embodiments, the antibody that blocks or disrupts PD-L2 is a human anti-PD-L2 antibody that is structurally related to mouse anti-human PD-L2 antibodies 24F.10C12, GF17.2C9, MIH37, 3.2, or TY25. In some embodiments, the antibody that blocks or disrupts PD-L2 comprises a heavy chain variable region sequence comprising SEQ ID NOS:7-11 and/or a light chain variable region sequence comprising SEQ ID NO:12-14. In some embodiments, the antibody that blocks or disrupts PD-L2 is a bispecific antibody. In some embodiments, the first agent is an antibody that disrupts or blocks RGMb. In some embodiments, the antibody that disrupts or blocks RGMb is a monoclonal antibody. In some embodiments, the antibody that blocks or disrupts RGMb is a humanized antibody. In some embodiments, the antibody that disrupts or blocks RGMb, comprises the heavy chain variable domain amino acid sequence encoded by SEQ ID NO: 17 and the light chain variable domain amino acid sequence encoded by SEQ ID NO: 16. In some embodiments, the antibody that blocks or disrupts RGMb is a bispecific antibody. In some embodiments, the second agent is an antibody. In some embodiments, the second agent is an antibody, a non-activating form of PD-L1, a fusion protein, a nucleic acid molecule that blocks PD-L1 transcription or translation, or a small molecule PD-L1 antagonist. In some embodiments, the second agent is an antibody that blocks PD-1. In some embodiments, the antibody that blocks PD-1 is a monoclonal antibody. In some embodiments, the antibody that blocks PD-1 is a humanized antibody. In some embodiments, the antibody that blocks PD-1 is a bispecific antibody. In some embodiments, the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188. In some embodiments, the second agent is an antibody that blocks PD-L1. In some embodiments, the antibody that blocks PD-L1 is a monoclonal antibody. In some embodiments, the antibody that blocks PD-L1 is a humanized antibody. In some embodiments, the antibody that blocks PD-L1 is a bispecific antibody. In some embodiments, the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the experimental design for identifying the mechanisms by which bacteria promote anti-tumor immunity. Mice are given antibiotics (0.5 mg/ml Vancomycin, 1 mg/ml Neomycin, 1 mg/ml metronidazole, 1 mg/ml Ampicillin) in drinking water 4 days before tumor implantation. On day zero, 2.5×10⁵ MC38 tumor cells are implanted subcutaneously in the abdomen of 6 week old female mice. On days 7, 10, 13, 16 mice are treated with 100 μg of isotype control or anti-PD-L1 by intraperitoneal injection. On day 7, half of the mice are orally gavaged with a slurry of Hmb feces and antibiotics are removed from the drinking water; the other half of the mice do not receive Hmb feces and continue with antibiotics in the drinking water for the remainder of the experiment. Tumors are measured on days 7, 10, 13, 16, 20, 23.

FIGS. 2A, FIG. 2B, and FIG. 2C show that mice treated with broad spectrum antibiotics (VNMA/ABX) for 17 days have an altered microbiota, referred to as dysbiosis. This microbiota is made up dominantly of a Proteobacteria, E. coli. Mice treated with VNMA, but then given an oral dose of a healthy human microbiota (Hmb) have a much more diverse microbiota. Each color represents a different species defined by 16S sequencing. Yellow represents E. coli.

FIG. 3 shows that mice (implanted with MC38 tumors) treated with VNMA do not respond to anti-PD-L1 therapy, whereas mice given an oral dose of Hmb are able to clear tumors with anti-PD-L1 therapy indicating that dysbiosis caused by VNMA treatment blocks the anti-tumor effects of anti-PD-L1 therapy.

FIGS. 4A-4B consists of two parts, A-B, and shows that 10 days (FIG. 4A) and 13 days (FIG. 4B) after MC38 tumor implantation, Hmb mice express significantly lower levels of PD-L2 on macrophages and dendritic cells compared to VNMA mice in the tumor draining lymph nodes (dLNs), indicating that PD-L2 over expression in dLNs from VNMA mice is involved in resistance to anti PD1/PD-L1.

FIG. 5 shows that VNMA-treated mice implanted with MC38 tumors do not respond to anti-PD-L1 or anti-PD-L2 alone. However, when given in combination, anti-PD-L1 and anti-PD-L2 therapy reverse the immuno-suppressive effects of dysbiosis and promote an anti-tumor response.

FIG. 6 shows that, whilst anti-PD-L1 alone is not effective, combined PD-L1 and PD-L2 blockade synergistically promotes anti-tumor response in germ-free (GF) mice (Germ-free mice are bred in isolators which fully block exposure to microorganisms, with the intent of keeping them free of detectable bacteria, viruses, and eukaryotic microbes) implanted with MC38 tumors.

FIGS. 7A-7C shows that combined PD-1 and PD-L2 blockage synergistically promotes anti-tumor responses in germ-free (FIG. 7A) and specific-pathogen-free (FIG. 7B and FIG. 7C) mice.

FIG. 8 shows that, whilst αPD-1 alone is not effective, combination between αPD-1 and αRGMb synergistically promotes anti-tumor response to immunotherapy in germ-free mice implanted with MC38 tumors.

FIG. 9 shows that, whilst αPD-L1 alone is not effective, combination between αPD-L1 and αRGMb synergistically promotes anti-tumor response to immunotherapy in germ-free mice implanted with MC3 8 tumors.

FIG. 10 shows the relative levels of over-expression of PD-L2 and RGMb molecules in lymph node dendritic cells isolated from antibiotic treated mice as opposed to mice having healthy human microbiota.

FIG. 11 shows that VNMA-treated mice implanted with MC38 tumors do not respond to anti-PD-L1 alone. However, when given in combination, anti-PD-L1 and anti-PD-L2 (2C9) therapy promotes a durable anti-tumor response over a period of 37 days.

FIG. 12 shows that αPD-L2 therapy combined with αPD-L1 increases survival compared to αPD-L1 therapy alone in VNMA antibiotic treated mice.

FIG. 13 shows that αPD-L2 therapy (either 3.2 or 2C9) combined with αPD-L1 therapy increases efficacy of αPD-L1 therapy alone in dysbiotic mice implanted with B16-OVA tumors (model for cancer immunotherapy, expressing ovalbumin OVA in order to facilitate strong immune responses to tumor antigens).

FIG. 14 shows that αPD-L2 therapy (either 3.2 or 2C9) combined with αPD-L1 increases survival compared to αPD-L1 therapy alone in dysbiotic mice implanted with B16-OVA tumors (10 mice per group).

FIG. 15 shows that αPD-L2 therapy (either 3.2 or 2C9) combined with αPD-L1 increases efficacy and tumor clearance of αPD-L1 therapy alone in germ free mice implanted with MC38 tumors (4-5 mice per group).

FIGS. 16A-16L shows (FIG. 16A) schematic of the experimental setups. Tumors were implanted subcutaneously at day 0 and antibodies were injected on days 7, 10, 13, 16 for all conditions. For GF+HMB mice, GF mice were orally gavaged with HMB stock 7 days before tumor implantation. GF and GF+Hmb experiments were performed in gnotobiotic isolators (ovals). For ABX mice, Vancomycin, Neomycin, Metronidazole, and Ampicillin were introduced in the drinking water 4 days before tumor implantation and remained in the drinking water for the duration of the experiment. For ABX+HMB mice, Vancomycin, Neomycin, Metronidazole, and Ampicillin were introduced in the drinking water 4 days before tumor implantation and removed from the drinking water at day 7 and mice were orally gavaged with HMB stock. s.c. subcutaneously, i.p. intraperitoneally, p.o. orally, i.g. intragastric (oral gavage). MC38 tumor growth in specific-pathogen-free (FIG. 16B) GF (FIG. 16C) ABX and ABX+HMB (FIG. 16D) GF+HMB (FIG. 16E). Schematic of experimental set up for dLN and tumor samples at days 10-16 (FIG. 16F). Heatmaps showing relative gene expression of co-signaling molecules most up or downregulated in HMB Responder mice in tumor draining lymph node dendritic cells (FIG. 16G) tumor infiltrating dendritic cells (FIG. 16H) tumor draining lymph node CD8+ T cells (FIG. 16I) and tumor infiltrating CD8⁺ T cells (FIG. 16J). MC38 tumor cells were implanted subcutaneously in ABX or ABX+HMB mice (FIG. 16K) and treated according to schematic A. Significance measured by Two Way ANOVA and Tukey's multiple comparisons test **<0.003. Individual growth curves of MC38 tumors in ABX+HMB mice treated with αPD-L1 from D shown in (FIG. 16L); 1 mouse did not respond to αPD-L1 treatment “NR” and 4 mice responded “R”. Representative example from 8 experiments 4-10 mice per group.

FIGS. 17A-17L shows status of immune cells from ABX and ABX+HMB mice treated with isotype or αPD-L1 at days 7 and 10, and sacrificed 13 days after tumor implantation. Numbers of CD45⁺ cells (FIG. 17A), CD8⁺ T cells (FIG. 17B), CD4⁺ T cells (FIG. 17C), MHCII⁺ CD11b⁺ cells (FIG. 17D), and MHCII⁺ CD11c⁺ cells (FIG. 17E) in tumor draining lymph nodes (dLNs). Percent of PD-1 expression on CD8⁺ T cells in tumors, dLNs, and mesenteric lymph nodes (MLNs) (FIG. 17F). Percent Tim3⁺ of PD-1⁺ CD8⁺ T cells in Tumors, dLNs, and MLNs (FIG. 17G) Percent CD44+ expression on PD1⁺ CD8⁺ T cells in Tumors, dLNs, and MLNs (FIG. 1711) Percent IFNγ+ CD8+ T cells in tumors, dLNs, and MLNs (FIG. 17I) Percent PD-L2 on MHCII+ CD11c⁺ MHCII⁺ CD11b⁺, and CD8⁺ T cells in dLNs (FIG. 17J) tumors (FIG. 17K), and MLNs (FIG. 17L). Significance determined by one-way ANOVA and Bonferroni's multiple comparisons test.

FIGS. 18A-18D shows microbiota impact on co-stimulatory and co-inhibitory protein expression on antigen presenting cells in the tumor draining lymph nodes of ABX vs ABX+HMB mice treated with isotype. Expression of PD-L1 (FIG. 18A) CD80 (FIG. 18B) CD86 (FIG. 18C) ICOSL (FIG. 18D) on CD11c⁺ MHCII⁺ and CD11b⁺ MHCII⁺ cells in draining lymph nodes of ABX and ABX+HMB mice treated with isotype. Significance measured by one-way ANOVA and Bonferroni's multiple comparisons. **** P<0.0001, *** P<0.001, ** P<0.01, *P<0.05.

FIGS. 19A-19E shows co-stimulatory and co-inhibitory protein expression on antigen presenting cells in tumors of ABX vs ABX+HMB mice treated with isotype. Expression of PD-L2 (FIG. 19A) PD-L1 (FIG. 19B) CD80 (FIG. 19C) CD86 (FIG. 19D) ICOSL (FIG. 19E) on CD11c⁺ MHCII⁺ and CD11b⁺ MHCII⁺ cells in tumors of ABX and ABX+HMB mice treated with isotype. Significance measured by one-way ANOVA and Bonferroni's multiple comparisons. **** P<0.0001, *** P<0.001, ** P<0.01, *P<0.05.

FIGS. 20A-20B shows suppression of PD-L2 by the microbiota. Expression of PD-L2 on CD11c⁺ MHCII⁺ and CD11b⁺ MHCII⁺ cells in draining lymph nodes at day 10 post implantation (p.i.) of ABX vs ABX+HMB mice treated with isotype (FIG. 20A) and at day 11 p.i. of GF vs SPF (specific-pathogen-free) mice treated with isotype (FIG. 20B). Significance measured by one-way ANOVA and Bonferroni's multiple comparisons. **** P<0.0001, *** P<0.001, ** P<0.01, *P<0.05.

FIGS. 21A-21E shows regulation of anti-tumor immunity in GF mice by RGMb (FIG. 21A) CD4⁺ T cells, CD8⁺ T cells, CD11c⁺ MHCII class II⁺ cells and CD11b+ cells were sorted from MC38 tumors of GF and SPF mice at post tumor implantation (p.i.) day 11, then, the levels of RGMb mRNA transcripts were quantified by qPCR. (FIG. 21B) Surface expression of RGMb protein on tumor-infiltrating leukocytes isolated from MC38 tumors at p.i. day 13 was measured by flow cytometry using monoclonal antibody (clone 9D3) against RGMb. Representative histograms of expression of RGMb on CD8⁺ T cells (upper left) and CD11c+MHC class II⁺ (lower left) in GF (black) and SPF (red) mice are shown and frequencies of RMGB⁺ cells within indicated cell populations quantified (right). (FIG. 21C-E) MC38 tumors were harvested from GF mice treated with indicated antibodies at p.i. day 11. (FIG. 21C) Frequencies of PD-1⁺ cells among CD8⁺ tumor-infiltrating lymphocytes and ICOS⁺ cells within T-bet⁺ CD8⁺ T cells from tumors (FIG. 21D) and tumor draining lymph nodes (FIG. 21E). Significance determined by one-way ANOVA and Bonferroni's multiple comparisons test for A-L.

FIGS. 22A-22D shows RGMb expression is modulated by the gut microbiota. Relative mRNA expression (FIG. 22A) and protein surface expression (FIG. 22B-D) of RGMb in CD4⁺ T cells, CD8⁺ T cells, CD11c⁺ MHCII⁺ and CD11b⁺ cells from tumor draining lymph nodes of GF and SPF mice at day 11 p.i. The levels of rgmb transcripts were normalized to expression of an internal control gene 18S rRNA. (FIG. 22B) Frequencies of RGMb⁺ cells were measured using 9D3 clone. Alternatively, geometric Mean Fluorescent Intensity (gMFI) of RGMb in indicated populations from tumor (FIG. 22C) and tumor draining lymph nodes (FIG. 22D) was assessed using αRGMb polyclonal antibody. Each group has 4-5 replicates. Significance measured by unpaired Student's t-test. **** P<0.0001, *** P<0.001, ** P<0.01, *P<0.05.

FIG. 23 shows CD8⁺ T cells are required for combined treatment of αPD-L1 and αPD-L2. MC38 tumor cells were implanted subcutaneously in β2m−/− mice (B2M KO), β2m+/− (Het), and WT littermate controls. n=3-5 mice per group. Significance measured by one-way ANOVA and Bonferroni's multiple comparisons. **** P<0.0001, *** P<0.001, ** P<0.01, *P<0.05.

FIGS. 24A-24E shows RGMb disruption induces a minor change in the number of tumor-infiltrating T cells. Total cell number of CD8⁺ T cells (FIG. 24A), CD4⁺ T cells (FIG. 24B) and CD4⁺ regulatory T (Treg) cells (FIG. 24C) per gram of tumors of GF mice treated with indicated antibodies at day 11 p.i. (FIG. 24D) CD8:Treg ratio in tumor burdens was quantified. (FIG. 24E) The absolute numbers of CD8⁺ T cells and CD4⁺ T cells in tumor draining lymph nodes were measured. Significance measured by one-way ANOVA and Bonferroni's multiple comparisons. **** P<0.0001, *** P<0.001, ** P<0.01, *P<0.05.

FIGS. 25A-25C shows RGMb does not alter expression of T cell exhaustion related markers and ICOS in tumor-infiltrating CD8⁺ T cells. Frequencies of Tim-3⁺ (FIG. 25A), LAG-3⁺ (FIG. 25B) and ICOS⁺ (FIG. 25C) populations among CD8⁺ T cells isolated from tumors of GF mice treated with indicated antibodies at day 11 p.i. were measured. Significance measured by one-way ANOVA and Bonferroni's multiple comparisons. **** P<0.0001, *** P<0.001, ** P<0.01, *P<0.05.

FIG. 26A-26B shows combined anti-RGMb and anti-PD-L1 treatment synergistically up-regulates PD-1 and ICOS on CD4⁺ T cells in tumor draining lymph nodes. Frequencies of PD-1⁺ (FIG. 26A) and ICOS⁺ (FIG. 26B) populations among CD4⁺ T cells in tumor draining lymph nodes of GF mice treated with indicated antibodies at day 11 p.i. were quantified. Significance measured by one-way ANOVA and Bonferroni's multiple comparisons. **** P<0.0001, *** P<0.001, ** P<0.01, *P<0.05.

FIG. 27 shows RGMb disruption potentiates pro-inflammatory cytokine TNF-α production by CD4⁺ tumor-infiltrating T cells. Tumor-infiltrating lymphocytes isolated from tumors of GF mice treated with indicated antibodies at day 11 p.i. were stimulated with PMA/Ionomycin for 5 hours. Frequencies of TNF-α producing cells among CD4⁺ T cell population were measured by intracellular staining and flow cytometry Significance measured by one-way ANOVA and Bonferroni's multiple comparisons. **** P<0.0001, *** P<0.001, ** P<0.01, *P<0.05.

FIGS. 28A-28D shows PD-L1 blockade and RGMb disruption do not increase expression of co-stimulatory ligands. Frequencies of CD80⁺ (FIG. 28A), CD86⁺ (FIG. 28B), CD40⁺ (FIG. 28C) and PD-L2⁺ (FIG. 28D) cells among CD11c⁺ MHC class II⁺ population in tumors of GF mice treated with indicated antibodies were graphed. Significance measured by one-way ANOVA and Bonferroni's multiple comparisons. **** P<0.0001, *** P<0.001, ** P<0.01, *P<0.05.

FIG. 29A and FIG. 29B show that αPD-L2 therapy (3.2) combined with αPD-L1 increases survival compared to αPD-L1 therapy alone in mice treated with broad spectrum antibiotics implanted with MB49 tumors.

FIG. 30 shows that αPD-L2 therapy (3.2) combined with αPD-L1 increases survival compared to αPD-L1 therapy alone and reduces size of tumors in mice treated with broad spectrum antibiotics and implanted with MB49 tumors.

FIG. 31 show that αPD-L2 therapy (3.2) combined with αPD-1 increases survival compared to αPD-1 therapy alone in mice treated with broad spectrum antibiotics and implanted with MB49 tumors.

FIG. 32 shows that αPD-L2 therapy (3.2) combined with αPD-1 increases survival compared to αPD-1 therapy alone in mice treated with broad spectrum antibiotics and implanted with MB49 tumors.

FIG. 33 shows an experimental timeline in which germ free mice were orally inoculated with stool stock from three melanoma patients to investigate the effect of immune checkpoint inhibitors on patients with gut microbiota altered by melanoma.

FIGS. 34A, FIG. 34B and FIG. 34C show that combined anti-PD-1 and anti-PD-L2 therapy promotes a more durable anti-tumor response than anti-PD-1 therapy alone in mice inoculated with stool from melanoma patients.

FIG. 35A and FIG. 35B show that combined anti-PD-1 and anti-PD-L2 therapy promotes a more durable anti-tumor response than anti-PD-1 therapy alone in mice inoculated with stool from melanoma patients.

FIG. 36A and FIG. 36B show that combined anti-PD-1 and anti-PD-L2 therapy promotes a more durable anti-tumor response than anti-PD-1 therapy alone in mice inoculated with stool from melanoma patients.

FIG. 37A and FIG. 37B show that combined anti-PD-1 and anti-PD-L2 therapy promotes a more durable anti-tumor response than anti-PD-1 therapy alone in mice inoculated with stool from melanoma patients.

This patent or application file contains at least one drawing executed in color. Copies of this patent or patent application publication with color drawings will be provided by the Office upon request and payment of necessary fee.

### DETAILED DESCRIPTION

Immune checkpoint blockade, or immunotherapy, is a novel therapeutic approach that reinvigorates tumor-specific T cells to efficiently kill cancer cells by blocking inhibitory pathways in T cells including CTLA-4 and PD-1. In recent years, antibodies against immune checkpoint molecules have attracted attention as new therapeutic agents for cancer. Immune checkpoint inhibitors promote the activation of T cells by inhibiting a molecule that suppresses the activation and function of T cells, and enhances the antitumor response of the T cells. In a treatment with an immune checkpoint inhibitor, cancer is eliminated by activating the immune state of the living body.

Despite the clinical success of immune checkpoint blockade-based drugs, for example drugs which modulate the anti-PD-1/anti-PD-L1 pathway, a significant fraction of cancer patients do not respond to or fail the therapy.

Described herein, in one aspect, is a method for treating a cancer in an individual that has failed an anti-PD1/PD-L1 therapy, comprising a) selecting an individual that has failed a prior anti-PD1/PD-L1 therapy; and b) administering to the individual i) a first agent that blocks or disrupts PD-L2, RGMb, or a combination thereof, and ii) a second agent that blocks or disrupts PD-L1, PD-1 or a combination thereof.

Described herein, in another aspect, is a therapeutic composition for treating a cancer in an individual comprising: a) a first agent that blocks or disrupts PD-L2, RGMb, or a combination thereof, and b) a second agent that blocks or disrupts PD-L1, PD-1 or a combination thereof.

Described herein, in another aspect is a kit for treating a cancer in an individual comprising: a) a first agent that blocks or disrupts PD-L2, RGMb, or a combination thereof; b) a second agent that disrupts PD-L1, PD-1 or a combination thereof and c) instructions for use of the first agent and the second agent in treating a cancer in an individual.

A. Therapeutic Methods

Described herein, in one aspect, is a method for treating cancer in an individual that has failed an anti-PD1/PD-L1 therapy, comprising a) selecting an individual that has failed a prior anti-PD1/PD-L1 therapy; and b) administering to the individual i) a first agent that blocks or disrupts PD-L2, RGMb, or a combination thereof, and ii) a second agent that blocks or disrupts PD-L1, PD-1 or a combination thereof.

### Patient Selection

In some examples, methods described herein comprise selecting an individual that has failed an anti-PD-1/PD-L1 therapy. In some examples, the anti-PD-1/PD-L1 therapy is an anti-PD-1/PD-L1 therapy administered to treat any of the indications described herein. In some examples, the failed anti-PD-1/PD-L1 therapy is administered to treat a cancer. In some examples, the cancer comprises a solid tumor. In some examples, the failed anti-PD-1/PD-L1 therapy comprises administering to an individual an agent that disrupts the interaction between PD-1 and PD-L1. In some examples, the failed therapy treatment comprises administering to an individual at least one anti-PD-1 agent or one anti-PD-L1 agent. In some examples, the at least anti-PD-1 or anti-PD-L1 agent is selected from the group consisting of an antibody or antigen binding fragment thereof, a peptide, a small molecule, or an inhibitory nucleic acid. In some examples, the at least one anti-PD-1 agent is selected from the group consisting of cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188. In some examples, the list of anti-PD-L1 agents is selected from the group consisting of atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244. In some examples, the failed therapy comprises administering the at least one anti-PD-1 agent or anti-PD-L1 agent systemically, locally, or a combination thereof. In some examples, an individual is considered to have failed an anti-PD-1/PD-L1 therapy if the treated cancer is resistant to therapy, if the treated cancer has no response or an incomplete response (e.g., a response that is less than a complete remission) to the therapy, if the treated cancer progresses or relapses after the therapy, if the individual that initially responds to therapy but develops a resistance to the therapy, or if the individual has been taken off of the therapy due to intolerance to the therapy (for example, due to toxicity of the therapy in view of the individual's age or condition).

In some examples, the individual that has failed the anti-PD-1/PD-L1 therapy has dysbiosis. Dysbiosis refers to any altered state of microbiota of the gastrointestinal (GI) tract. As used herein, “dysbiosis” includes patients that have been treated with antibiotics, received chemotherapy, or have or have had conditions known to alter the microbiome such as: intestinal infections, ulcerative colitis, Crohn's disease, irritable bowel syndrome, colon cancer, dramatic changes or significant changes in diet (for example extended hospital stays). In a normal distribution of bacterial phlya in the gut, Bacteroidetes and Firmicutes are dominant. In some examples, a patient has dysbiosis if the gastrointestinal microbiota of the subject is comprised dominantly of E. coli or bacteria from other phyla instead of being dominantly comprised of Bacteroidetes and/or Firmicutes bacteria. Additionally, patients have dysbiosis if they have had their microbiome sequenced at different time points and the microbiome makeup has changed. For example, the gastrointestinal microbiota of the subject may comprise at least 50%, at least 60%, at least 70%, at least 80%, or at least 90% E. coli or other bacteria from other phyla that is not Bacteroidetes and/or Firmicutes. The gastrointestinal microbiota of the subject may have an imbalance of the normal distribution of bacterial phyla in the gut. Dysbiosis includes any microbiota profile typical of a patient with cancer, and/or a microbiota profile not typically seen in patients without cancer. For example, imbalance in the GI tract of a subject with dysbiosis includes a higher level of bacteria that are not Bacteroidetes and Firmicutes in the GI tract of the subject when compared to the level of other phyla that is not Bacteroidetes and Firmicutes in the GI tract of a subject without cancer, or when compared to the average or median level of other phyla that is not Bacteroidetes and Firmicutes in the GI tract of a population of subjects without cancer. Dysbiosis may also refer to a lack of microbiota diversity in the GI tract. Dysbiosis includes the altered GI microbiota typically found in an individual after antibiotic administration. Dysbiotic patients may be identified, for example, by the symptoms of dysbiosis, such as diarrhea, constipation, abdominal cramping, loose stool, and/or abnormal amounts of gas or bloating. Some patients may be assumed to be dysbiotic because of previous administration of cancer or antibiotic treatments known to cause dysbiosis. Dysbiotic patients may be identified, for example, by the symptoms of dysbiosis, such as diarrhea, constipation, abdominal cramping, loose stool, and/or abnormal amounts of gas or bloating. Some patients may be assumed to be dysbiotic because of previous administration of cancer or antibiotic treatments known to cause dysbiosis. In some instances, the dysbiosis causes, at least partially, an increase in immunological tolerance in the individual. In some examples, the dysbiosis, causes, at least partially, an increase in PD-L2 expression in tumor cells or antigen presenting cells in the individual. Without being limited to any theory, in some examples, an increase in PD-L2 expression in tumor cells or antigen presenting cells caused by the dysbiosis impairs the benefit of an immune checkpoint blockade (e.g., the PD-1/PD-L1 blockade induced by the anti-PD-1/anti-PD-L1 therapy, or a blockade induced by any other immune checkpoint inhibitor provided herein) that has failed in the individual.

In some examples, the combination of the first agent and the second agent results in an anti-tumor response, where an anti-tumor response was not caused by the failed PD-1/PD-L1 therapy. In some examples, the combination of the first agent and the second agent results in a synergistic effect in that the combination achieves at least one of: a greater therapeutic effect (i.e., more efficacious) than the additive therapeutic effect obtained by administration of the first or second agent alone, a greater therapeutic effect than achieved by administration of a higher dose of the first or second agent alone, a similar or greater therapeutic effect but with a decrease in adverse events or side effects relative to that observed by administration of the first or second agent alone (i.e., improved therapeutic window), or increased duration of effects, or a similar or greater therapeutic effect at a smaller dose of one or both of the first or second agents. or a combination thereof. In some examples, the synergistic effect is increased survival time, increased tumor stability or volume reduction, or increased anti-tumor activity as compared to single agent therapy alone.

In some examples, the combination of the first agent and the second agent yields improved anti-tumor results as compared to that produced by the PD-1/PD-L1 therapy. For example, the combination of the first agent and the second agent increases anti-tumor activity, increased survival, or increased tumor stability as compared to either agent alone. In some embodiments, tumor reduction is at least 1-fold, 2-fold, 3-fold, 4-fold, 5-fold, 6-fold, 7-fold, 8-fold, 9-fold, or up to 10-fold greater as compared to administration of a PD-1/PD-PL1 therapy alone. In some embodiments, survival is at least 1-fold, 2-fold, 3-fold, 4-fold, 5-fold, 6-fold, 7-fold, 8-fold, 9-fold, or up to 10-fold greater as compared to administration of the PD-1/PD-L1 therapy alone. In some embodiments, survival is increased from about 1 week, 1 month, 2 months, 3 months, 4 months, 5 months, 6 months, 7 months, 8 months, 1 year, 2 years, 3 years, 4 years, 5 years, 10 years, 15 years, 20 years, or up to about 30 years more as compared to administration of either agent alone.

### Antibody Agents

In certain aspects, the methods disclosed herein comprise administering to the individual i) a first agent that blocks or disrupts PD-L2, RGMb, or a combination thereof, and ii) a second agent that blocks or disrupts PD-L1, PD-1 or a combination thereof. In certain examples, the first agent and/or the second agent comprises an antibody or antigen binding fragment thereof that binds specifically to PD-L2 (e.g., a first agent), RGMb (e.g., a first agent), PD-1 (e.g., a second agent) or PD-L1 (e.g., a second agent). In some examples, the first agent and/or the second agent comprises an antibody that disrupts the molecules disclosed herein. Such antibodies can be polyclonal or monoclonal and can be, for example, murine, chimeric, humanized or fully human. In some examples, the antibody is bispecific (e.g., bispecific for PD-L2 and RGMb).

Polyclonal antibodies can be prepared by immunizing a suitable subject (e.g., a mouse) with a polypeptide antigen (e.g., a polypeptide having a sequence of PD-L2, RGMb, or a fragment thereof). The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal specific for a receptor or ligand provided herein can be identified and isolated by screening a recombinant combinatorial immunoglobulin library (e.g., an antibody phage display library or an antibody yeast display library) with the appropriate polypeptide to thereby isolate immunoglobulin library members that bind the polypeptide.

Additionally, recombinant antibodies specific for a receptor or ligand provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a receptor or ligand provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In some examples, the first or second agent is a composite antibody, i.e., an antibody which has variable regions comprising germline or non-germline immunoglobulin sequences from two or more unrelated variable regions. A composite, human antibody can be an antibody which has constant regions derived from human germline or non-germline immunoglobulin sequences and variable regions comprising human germline or non-germline sequences from two or more unrelated human variable regions. A composite, human antibody may exhibit lowered antigenicity in the human body.

In some examples, the first or second agent is a dual binding monoclonal antibody or antigen-binding fragment thereof that binds to both PD-L1 and PD-L2. In some examples, the dual binding monoclonal antibody or antigen-binding fragment thereof is produced by a hybridoma. In some examples, the dual binding monoclonal antibody or antigen-binding fragment thereof binds to the peptide sequence CFTVTVPKDLYVVEYGSN (SEQ ID NO: 1) or CYRSMISYGGADYKRITV (SEQ ID NO: 2). In some examples, the dual binding monoclonal antibodies are produced by a hybridoma. In some examples, dual binding agent comprises a) a heavy chain variable region sequence selected from the group consisting of SEQ ID NOS: 3 and 5, or a sequence with at least about 95% homology to a heavy chain variable region sequence selected from the group consisting of SEQ ID NOS: 3 and 5; and a light chain variable region sequence selected from the group consisting of SEQ ID NOS: 4 and 6, or a sequence with at least about 95% homology to a light chain variable region sequence selected from the group consisting of SEQ ID NOS: 4 and 6.

In some examples, the first agent that blocks or disrupts, PD-L2, RGMB or a combination thereof is a composite antibody in which a non-human antibody (e.g., a mouse anti-human PD-L2 antibody, such as 24F.10C12) is used to create a structurally related human anti-human PD-L2 antibody that retains at least one functional property of the non-human antibody, such as binding to PD-L2. SEQ ID NOS: 7-11 comprises the sequences of composite, human heavy chain variable region sequences designed to correspond to that of the mouse anti-human PD-L2 antibody, 24F.10C12. SEQ ID NOS: 12-15 comprise the sequences of composite, human light chain variable region sequences designed to correspond to that of the mouse anti-human PD-L2 antibody, 24F.10C12. In some examples, the agent comprises an isolated antibody or antigen-binding fragment thereof comprising a) a heavy chain variable region sequence selected from the group consisting of SEQ ID NOs: 7-11, or a sequence with at least about 95% homology to a heavy chain variable region sequence selected from the group consisting of SEQ ID NOs: 7-11; and a light chain variable region sequence selected from the group consisting of SEQ ID NOs: 12-15, or a sequence with at least about 95% homology to a light chain variable region sequence selected from the group consisting of SEQ ID NOs: 12-15.

In some examples, the first agent that blocks or disrupts, PD-L2, RGMB, or a combination thereof, is a monoclonal antibody, or fragment thereof produced by a hybridoma. In some examples, monoclonal antibody, or fragment thereof, produced by a hybridoma is rat monoclonal antibody, clone TY25, mouse monoclonal antibody, clone 3.2. mouse monoclonal antibody, clone MIH37, mouse monoclonal antibody, clone GF17.229, or rat anti-RGMB antibody, clone BFH-5C9. In some examples, the first agent is an antibody that binds to RGMB comprising a heavy chain variable region sequence comprising SEQ ID NO: 17, or a sequence with at least about 95% homology to a heavy chain variable region sequence selected from the group consisting of SEQ ID NOs: 17; and a light chain variable region sequence comprising SEQ ID NO: 16, or a sequence with at least about 95% homology to a light chain variable region sequence comprising SEQ ID NO: 16.

In some examples, the PD-L2 antibodies are those found in patents and published applications such as: U.S. Pat. Nos. 9,845,356, 10,370,448, US Patent Publication 2018/0002422, WO Pat. Publication WO2002000730, and US Patent Publication 2018/0258171, hereby incorporated by reference in their entireties.

In some examples, the first agent that blocks or disrupts PD-1, PD-L1, or a combination thereof is an antibody that blocks PD-1. In some examples, the antibody that blocks PD-1 is selected from the antibody that blocks PD-1 may be selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188.

In some examples, the first agent that blocks or disrupts PD-1, PD-L1, or a combination thereof is an antibody that blocks PD-L1. The antibody that blocks PD-L1 may be selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

### Proteins

In certain aspects, the methods disclosed herein comprise administering to the individual i) a first agent that blocks or disrupts PD-L2, RGMb, or a combination thereof, and ii) a second agent that blocks or disrupts PD-L1, PD-1 or a combination thereof. In some examples, the first agent and/or the second agent is a polypeptide that specifically binds to PD-L2, RGMb, PD-1, or PD-L1. In some examples, the polypeptides disrupt PD-L2, RGMb, PD-1, or PD-L1. In some examples, the polypeptides disrupt the interaction between PD-L2 and RGMb or disrupt the interaction between PD-1 and PD-L1.

In some examples, the polypeptides and proteins described herein are isolated from cells or tissue sources by an appropriate purification scheme using standard protein purification techniques. In some examples, polypeptides and proteins described herein are produced by recombinant DNA techniques. In some examples, polypeptides described herein can be chemically synthesized using standard peptide synthesis techniques.

In some examples provided herein are chimeric or fusion proteins. As used herein, a “chimeric protein” or “fusion protein” comprises a polypeptide or protein described herein linked to a distinct polypeptide to which it is not linked in nature. For example, the distinct polypeptide can be fused to the N-terminus or C-terminus of the polypeptide either directly, through a peptide bond, or indirectly through a chemical linker. In some examples, the peptide described herein is linked to an immunoglobulin constant domain (e.g., an IgG constant domain, such as a human IgG constant domain).

A chimeric or fusion polypeptide described herein can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The polypeptides and proteins described herein can be produced in prokaryotic or eukaryotic host cells by expression of polynucleotides encoding a polypeptide(s) described herein. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

In some examples, the first or second agent comprises peptide antagonist NP-12 [Ser-Asn-Thr-Ser-Glu-Ser-Phe-Lys(Ser-Asn-Thr-Ser-Glu-Ser-Phe)-Phe-Arg-Val-Thr-Gln-Leu-Ala-Pro-Lys-Ala-Gln-Ile-Lys-Glu-NH2] synthesized according to the processes described in Example 2 of U.S. Pat. No. 8,907,053.

In some examples, the first or second agent comprises peptide fusion protein AMP-224, an anti-PD-1 recombinant fusion protein composed of the extracellular domain of the human programmed cell death 1 ligand 2 (PD-L2) fused to the Fc domain of human immunoglobulin G1, which binds to PD-1 on the cell surface of T cells. See Charalampos et al, “A Pilot Study of the PD-1 Targeting Agent AMP-224 Used With Low-Dose Cyclophosphamide and Stereotactic Body Radiation Therapy in Patients With Metastatic Colorectal Cancer,” Clinical Colorectal Cancer, Vol. 18, Is. 4, 12-2019, Pages 349-360.

### Small Molecule Agents

In certain aspects, the methods disclosed herein comprise administering to the individual i) a first agent that blocks or disrupts PD-L2, RGMb, or a combination thereof, and ii) a second agent that blocks or disrupts PD-L1, PD-1 or a combination thereof. In some examples, the first agent and/or the second agent is a small molecule agent that specifically binds and disrupts PD-L2, RGMb, PD-1 or PD-L1. The agent may be a small molecule that disrupts the interaction between PD-L2/RGMb or PD-1/PD-L1.

In some examples, the first and/or the second agent is CA-170. Musielak et al, “A Potent Small-Molecule PD-L1 Inhibitor or Not?” Molecules 2019, 24, 2804.

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

### Interfering Nucleic Acid Agents

In certain aspects, the methods disclosed herein comprise administering to an individual that has failed an anti-PD-L1/anti-PD-1 treatment i) a first agent that blocks or disrupts PD-L2, RGMb, or a combination thereof, and ii) a second agent that blocks or disrupts PD-L1, PD-1 or a combination thereof. In some examples, the first agent and/or the second agent is an interfering nucleic acid agent that disrupts PD-L2, RGMb, PD-1 or PD-L1. The agent may be an interfering nucleic acid agent that disrupts the interaction between PD-L2/RGMb or PD-1/PD-L1.

In certain examples, interfering nucleic acid molecules that selectively target a product of a gene that encodes for PD-L2 or RGMb. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some examples, the interfering nucleic acid molecule is double-stranded RNA. The double-stranded RNA molecule may have a 2 nucleotide 3′ overhang. In some examples, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™. has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases 51 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain examples, a viral, retroviral or lentiviral vector is used. In some examples the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some examples, the interfering nucleic acids contain a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some examples, the interfering nucleic acid molecule is a siRNA molecule. Such siRNA molecules should include a region of sufficient homology to the target region, and be of sufficient length in terms of nucleotides, such that the siRNA molecule downregulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some examples, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some examples, the strand is at least 19 nucleotides in length. For example, each strand can be between 21 and 25 nucleotides in length. In some examples, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some examples, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some examples, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some examples, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional examples related to the shRNAs, as well as methods of designing and synthesizing such shRNAs, are described in U.S. patent application publication number 2011/0071208, the disclosure of which is herein incorporated by reference in its entirety for all purposes.

In some examples, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some examples, antisense oligonucleotide compounds are provided herein. In certain examples, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain examples, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some examples, the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is hereby incorporated by reference in its entirety.

In some examples, of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some examples, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some examples, the opsonization-inhibiting moiety is a PEG, PPG, or derivatives thereof. Liposomes modified with PEG or PEG-derivatives are sometimes called “PEGylated liposomes.”

### CRISPR/Gene Editing

In certain aspects, the methods disclosed herein comprise administering to an individual that has failed an anti-PD-L1/anti-PD-1 treatment i) a first agent that blocks or disrupts PD-L2, RGMb, or a combination thereof, and ii) a second agent that blocks or disrupts PD-L1, PD-1 or a combination thereof. In some examples, the first agent and/or the second agent is a gene editing agent that disrupts the interaction between PD-L2/RGMb or PD-1/PD-L1.

In some examples, the agent disclosed herein is an agent for genome editing (e.g., an agent used to delete at least a portion of a gene that encodes a PD-L2 or RGMb peptide). Deletion of DNA may be performed using gene therapy to knock-out or disrupt the target gene. As used herein, a “knock-out” can be a gene knock-down or the gene can be knocked out by a mutation such as, a point mutation, an insertion, a deletion, a frameshift, or a missense mutation by techniques known in the art, including, but not limited to, retroviral gene transfer. In some examples, the agent is a nuclease (e.g., a zinc finger nuclease or a TALEN). Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs). A TALEN is composed of a TALE DNA binding domain for sequence-specific recognition fused to the catalytic domain of an endonuclease that introduces double-strand breaks (DSB). The DNA binding domain of a TALEN is capable of targeting with high precision a large recognition site (for instance, 17 bp). Meganucleases are sequence-specific endonucleases, naturally occurring “DNA scissors,” originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

In another embodiment, the agent comprises a CRISPR-Cas9 guided nuclease and/or a sgRNA (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety). Like the TALENs and ZFNs, CRISPR-Cas9 interference is a genetic technique which allows for sequence-specific control of gene expression in prokaryotic and eukaryotic cells by guided nuclease double-stranded DNA cleavage. It is based on the bacterial immune system—derived CRISPR (clustered regularly interspaced palindromic repeats) pathway. In some examples, the agent is an sgRNA. An sgRNA combines tracrRNA and crRNA, which are separate molecules in the native CRISPR/Cas9 system, into a single RNA construct, simplifying the components needed to use CRISPR/Cas9 for genome editing. In some examples, the crRNA of the sgRNA has complementarity to at least a portion of a gene that encodes PD-L2 or RGMb (or a fragment thereof). In some examples, the sgRNA may target at least a portion of a gene that encodes a PD-L2 or RGMb protein.

### Dosages and Administration

In certain aspects, the methods disclosed herein comprise administering to an individual that has failed an anti-PD-L1/anti-PD-1 treatment i) a first agent that blocks or disrupts PD-L2, RGMb, or a combination thereof, and ii) a second agent that blocks or disrupts PD-L1, PD-1 or a combination thereof. In some examples, the first agent and/or the second agent is a gene editing agent that disrupts the interaction between PD-L2/RGMb or PD-1/PD-L1. In certain aspects, agents and/or compositions disclosed herein may be administered at a dose sufficient to achieve the desired result.

In certain examples, the method may comprise administering about 1 μg to about 1 gram of agent or composition to the subject, such as about 1 μg to about 1 mg, about 2 μg to about 2 mg, about 3 μg to about 3 mg, about 4 μg to about 4 mg, about 100 μg to about 2 mg, about 200 μg to about 2 mg, about 300 μg to about 3 mg, about 400 μg to about 4 mg, about 250 μg to about 1 mg, or about 250 μg to about 750 μg of the agent or composition. In some examples, the method may comprise administering about 25 about 50 about 75 μg/kg, about 100m/kg, about 125m/kg, about 150m/kg, about 175m/kg, about 200m/kg, about 225m/kg, about 250m/kg, about 275m/kg, about 300m/kg, about 325m/kg, about 350m/kg, about 375m/kg, about 400m/kg, about 425m/kg, about 450 μg/kg, about 475m/kg, about 500m/kg, about 600m/kg, about 650m/kg, about 700 μg/kg, about 750m/kg, about 800m/kg, about 850m/kg, about 900m/kg, about 950 μg/kg, about 1000m/kg, about 1200m/kg, about 1250m/kg, about 1300m/kg, about 1333 μg/kg, about 1350m/kg, about 1400m/kg, about 1500m/kg, about 1600m/kg, about 1750 μg/kg, about 1800m/kg, about 2000m/kg, about 2200m/kg, about 2250m/kg, about 2300 μg/kg, about 2333 μg/kg, about 2350m/kg, about 2400m/kg, about 2500m/kg, about 2667 μg/kg, about 2750m/kg, about 2800m/kg, about 3 mg/kg, about 3.5 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, or about 100 mg/kg. In some examples, the method may comprise administering about 1 mg/kg to about 10 mg/kg, about 10 mg/kg to about 20 mg/kg, about 20 mg/kg to about 50 mg/kg, about 50 mg/kg to about 100 mg/kg of the agent or composition. The dose may be titrated up or down following initial administration to any effective dose.

Immune checkpoint inhibitor dosing may follow any dosing regime or schedule known in the art. For example, dosing can be determined by cancer type or cancer disease stage, as well as the characteristics of the afflicted patient, such as weight, sex, ethnicity, and/or sensitivity to medication. Exemplary dosing regimes and schedules can be found at https://packageinserts.bms.com/pi/pi_opdivo.pdf; https://www.merck.com/product/usa/pi_circulars/k/keytruda/keytruda_pi.pdf; https://www.accessdata.fda.gov/drugsatfda_docs/label/2018/761069s0021b1.pdf; or https://www.accessdata.fda.gov/drugsatfda_docs/label/2017/761049s0001b1.pdf.

In some examples, the first agent and second agent are administered at the same time. In some examples, the first agent is administered 2, 3, 4, or 5 times as frequently as the second agent. In some examples, the second agent is administered 2, 3, 4, or 5 times as frequently as the first agent.

In some examples, administering an agent (e.g., a first and/or second agent) or composition to the subject comprises administering a bolus of the composition. The method may comprise administering the composition to the subject at least once per month, twice per month, three times per month. In certain examples, the method may comprise administering the composition at least once per week, at least once every two weeks, or once every three weeks. In some examples, the method may comprise administering the composition to the subject 1, 2, 3, 4, 5, 6, or 7 times per week.

### Additional Therapies

In certain aspects, the methods disclosed herein comprise administering to an individual that has failed an anti-PD-L1/anti-PD-1 treatment i) a first agent that blocks or disrupts PD-L2, RGMb, or a combination thereof, and ii) a second agent that blocks or disrupts PD-L1, PD-1 or a combination thereof. In some embodiments, the first and second agents described herein are administered in combination with an additional therapeutic agent described herein. Also described herein are therapeutic compositions, e.g., pharmaceutical compositions, for treating a cancer in an individual comprising: a) a first agent that blocks or disrupts PD-L2, RGMb, or a combination thereof, and b) a second agent that blocks or disrupts PD-L1, PD-1 or a combination thereof. In some examples, the agent or pharmaceutical composition is administered with an additional therapeutic agent. In some examples, the additional therapeutic agent is a chemotherapeutic agent. Exemplary chemotherapeutic agents include alkylating agents such as thiotepa and cyclophosphamide (Cytoxan™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phili); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (Taxol™, Bristol Meyers Squibb Oncology, Princeton, N.J.) and docetaxel (Taxoteret™, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™) letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above. In some examples, the additional therapeutic agent is an immune checkpoint inhibitor. Immune Checkpoint inhibition broadly refers to inhibiting the checkpoints that cancer cells can produce to prevent or downregulate an immune response. Examples of immune checkpoint proteins are CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof.

### Indications

In certain aspects, the methods disclosed herein comprise administering to an individual that has failed an anti-PD-L1/anti-PD-1 treatment i) a first agent that blocks or disrupts PD-L2, RGMb, or a combination thereof, and ii) a second agent that blocks or disrupts PD-L1, PD-1 or a combination thereof. In some examples, the first agent and/or the second agent is a gene editing agent that disrupts the interaction between PD-L2/RGMb or PD-1/PD-L1.

In some cases, the individual is (or is identified as) a non-responder, a partial responder, a relapser, or a combination thereof, to an anti-PD-1 therapy. In some cases, the individual is a non-responder to an anti-PD-1 therapy. In some instances, a non-responder to an anti-PD-1 therapy is an individual that has a cancer that does not respond to the anti-PD-1 therapy (e.g., is a stable cancer or a cancer that has stable progression). In some cases, a non-responder to an anti-PD1 therapy has a cancer that exhibits less than a 50%, 45%, 40%, 35%, 30%, 25%, 20%, 15%, 10%, 5%, or 0% reduction in tumor volume after receiving an anti-PD1 therapy. In some cases, a non-responder has a cancer that exhibits less than a 50% reduction in tumor volume after at least 10 days, 14 days, 18 days, 22 days, 26 days, 30 days, 34 days or 38 days of an anti-PD1 therapy. In some case, a non-responder has a cancer that exhibits less than a 50% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a non-responder has a cancer that exhibits less than a 40% reduction in tumor volume after at least 10 days, 14 days, 18 days, 22 days, 26 days, 30 days, 34 days or 38 days of an anti-PD-1 therapy. In some case, a non-responder has a cancer that exhibits less than a 40% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a non-responder has a cancer that exhibits less than a 30% reduction in tumor volume after at least 10 days, 14 days, 18 days, 22 days, 26 days, 30 days, 34 days or 38 days of an anti-PD-1 therapy. In some case, a non-responder has a cancer that exhibits less than a 30% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a non-responder has a cancer that exhibits less than a 20% reduction in tumor volume after at least 10 days, 14 days, 18 days, 22 days, 26 days, 30 days, 34 days or 38 days of an anti-PD-1 therapy. In some case, a non-responder has a cancer that exhibits less than a 20% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a non-responder has a cancer that exhibits less than a 10% reduction in tumor volume after at least 10 days, 14 days, 18 days, 22 days, 26 days, 30 days, 34 days or 38 days of an anti-PD1 therapy. In some case, a non-responder has a cancer that exhibits less than a 10% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a non-responder has tumor which is an “escaper tumor” from an anti-PD1 therapy. In some cases, an escaper tumor is a tumor which evades or is not reduced in size by an anti-PD1 therapy. In some cases, an escaper tumor is caused, at least in part, by dysbiosis. In some cases, dysbiosis inhibits the anti-tumor effect of anti-PD-1 therapy, thereby resulting in a “escaper tumor”. In some instances, the individual is a partial responder to an anti-PD-1 therapy. In some instances, a partial responder to an anti-PD-1 therapy is an individual having a cancer that exhibits a partial response to an anti-PD-1 therapy. In some cases, a partial responder to an anti-PD1 therapy has a cancer that exhibits less than a 90%, 85%, 80%, 75%, 70%, 65%, or 60% reduction in tumor volume after receiving an anti-PD1 therapy. In some cases, a non-responder has a cancer that exhibits less than a 90% reduction in tumor volume after at least 10 days, 14 days, 18 days, 22 days, 26 days, 30 days, 34 days or 38 days of an anti-PD-1 therapy. In some case, a partial responder has a cancer that exhibits less than a 90% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a partial responder has a cancer that exhibits less than a 80% reduction in tumor volume after at least 10 days, 14 days, 18 days, 22 days, 26 days, 30 days, 34 days or 38 days of an anti-PD-1 therapy. In some case, a partial responder has a cancer that exhibits less than a 80% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a partial responder has a cancer that exhibits less than a 70% reduction in tumor volume after at least 10 days, 14 days, 18 days, 22 days, 26 days, 30 days, 34 days or 38 days of an anti-PD-1 therapy. In some case, a partial responder has a cancer that exhibits less than a 70% reduction in tumor volume after at least 1 month, 2 months, 3 months, 4 months, 5 months, 6 months, 7 months, 8 months, 9 months, 10 months, 11 months, or 12 months of an anti-PD-1 therapy. In some cases, a partial responder has a cancer that exhibits less than a 60% reduction in tumor volume after at least 10 days, 14 days, 18 days, 22 days, 26 days, 30 days, 34 days or 38 days of an anti-PD-1 therapy. In some case, a partial responder has a cancer that exhibits less than a 60% reduction in tumor volume after at least 1 month, 2 months, 3 months, 4 months, 5 months, 6 months, 7 months, 8 months, 9 months, 10 months, 11 months, or 12 months of an anti-PD-1 therapy. In some instances, the individual is a relapser to an anti-PD-1 therapy. In some cases, a relapser experiences reappearance or an increase in a cancer or a tumor volume after an initial period of responsiveness to an anti-PD-1 therapy (e.g., a reduction in tumor volume or cancer cells below 50%, 45%, 40%, 35%, 30%, 25%, 20%, 15%, 10%, 5% or less).

In some cases, the individual is (or is identified as) a non-responder, a partial responder, a relapser, or a combination thereof, to an anti-PD-L1 therapy. In some cases, the individual is a non-responder to an anti-PD-L1 therapy. In some instances, a non-responder to an anti-PD-L1 therapy is an individual that has a cancer that does not respond to the anti-PD-L1 therapy (e.g., is a stable cancer or a cancer that has stable progression). In some cases, a non-responder to an anti-PD1 therapy has a cancer that exhibits less than a 50%, 45%, 40%, 35%, 30%, 25%, 20%, 15%, 10%, 5%, or 0% reduction in tumor volume after receiving an anti-PD1 therapy. In some cases, a non-responder has a cancer that exhibits less than a 50% reduction in tumor volume after at least 10 days, 14 days, 18 days, 22 days, 26 days, 30 days, 34 days or 38 days of an anti-PD1 therapy. In some case, a non-responder has a cancer that exhibits less than a 50% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a non-responder has a cancer that exhibits less than a 40% reduction in tumor volume after at least 10 days, 14 days, 18 days, 22 days, 26 days, 30 days, 34 days or 38 days of an anti-PD-L1 therapy. In some case, a non-responder has a cancer that exhibits less than a 40% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a non-responder has a cancer that exhibits less than a 30% reduction in tumor volume after at least 10 days, 14 days, 18 days, 22 days, 26 days, 30 days, 34 days or 38 days of an anti-PD-L1 therapy. In some case, a non-responder has a cancer that exhibits less than a 30% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a non-responder has a cancer that exhibits less than a 20% reduction in tumor volume after at least 10 days, 14 days, 18 days, 22 days, 26 days, 30 days, 34 days or 38 days of an anti-PD-L1 therapy. In some case, a non-responder has a cancer that exhibits less than a 20% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a non-responder has a cancer that exhibits less than a 10% reduction in tumor volume after at least 10 days, 14 days, 18 days, 22 days, 26 days, 30 days, 34 days or 38 days of an anti-PD1 therapy. In some case, a non-responder has a cancer that exhibits less than a 10% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a non-responder has tumor which is an “escaper tumor” from an anti-PD1 therapy. In some cases, an escaper tumor is a tumor which evades or is not reduced in size by an anti-PD1 therapy. In some cases, an escaper tumor is caused, at least in part, by dysbiosis. In some cases, dysbiosis inhibits the anti-tumor effect of anti-PD-L1 therapy, thereby resulting in a “escaper tumor”. In some instances, the individual is a partial responder to an anti-PD-L1 therapy. In some instances, a partial responder to an anti-PD-L1 therapy is an individual having a cancer that exhibits a partial response to an anti-PD-L1 therapy. In some cases, a partial responder to an anti-PD1 therapy has a cancer that exhibits less than a 90%, 85%, 80%, 75%, 70%, 65%, or 60% reduction in tumor volume after receiving an anti-PD1 therapy. In some cases, a non-responder has a cancer that exhibits less than a 90% reduction in tumor volume after at least 10 days, 14 days, 18 days, 22 days, 26 days, 30 days, 34 days or 38 days of an anti-PD-L1 therapy. In some case, a partial responder has a cancer that exhibits less than a 90% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a partial responder has a cancer that exhibits less than a 80% reduction in tumor volume after at least 10 days, 14 days, 18 days, 22 days, 26 days, 30 days, 34 days or 38 days of an anti-PD-L1 therapy. In some case, a partial responder has a cancer that exhibits less than a 80% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a partial responder has a cancer that exhibits less than a 70% reduction in tumor volume after at least 10 days, 14 days, 18 days, 22 days, 26 days, 30 days, 34 days or 38 days of an anti-PD-L1 therapy. In some case, a partial responder has a cancer that exhibits less than a 70% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some cases, a partial responder has a cancer that exhibits less than a 60% reduction in tumor volume after at least 10 days, 14 days, 18 days, 22 days, 26 days, 30 days, 34 days or 38 days of an anti-PD-L1 therapy. In some case, a partial responder has a cancer that exhibits less than a 60% reduction in tumor volume after at least 1 month, 2 months, 3 months, 4 months, 5 months, 6 months, 7 months, 8 months, 9 months, 10 months, 11 months, or 12 months of an anti-PD-L1 therapy. In some instances, the individual is a relapser to an anti-PD-L1 therapy. In some cases, a relapser experiences reappearance or an increase in a cancer or a tumor volume after an initial period of responsiveness to an anti-PD-L1 therapy (e.g., a reduction in tumor volume or cancer cells below 50%, 45%, 40%, 35%, 30%, 25%, 20%, 15%, 10%, 5% or less).

In some examples, the methods described herein may be used to treat any cancerous or pre-cancerous tumor. In some examples, the cancer includes a solid tumor. Cancers that may be treated by methods and compositions provided herein include, but are not limited to, cancer cells from the bladder, blood, bone, bone marrow, brain, breast (e.g., estrogen receptor (ER)-positive breast cancer, triple negative breast cancer, or HER2 positive breast cancer), colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some examples, the methods described herein are used to treat a head and neck cancer. In some examples, the methods described herein are used to treat head and neck squamous cell carcinoma. In some examples, the cancer is a PD-L2 expressing cancer. In some examples, the PD-L2 expressing cancer is esophageal squamous cell carcinoma, head and neck squamous cell carcinoma, or a combination thereof. In some examples, the cancer is an RGMb-expressing cancer.

In some examples, the subject has cancer. In some examples, the cancer comprises a solid tumor. In some examples, the tumor is immunogenic or highly immunogenic. In some examples, the tumor is resistant to immunotherapy. In some examples, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor. In some embodiments, the tumor cells express RGMb, PD-L2 or a combination thereof.

B. Therapeutic Compositions

Described herein, in one aspect, is a therapeutic composition, e.g., a pharmaceutical composition, for treating a cancer in an individual comprising: a) a first agent that blocks or disrupts PD-L2, RGMb, or a combination thereof, and b) a second agent that blocks or disrupts PD-L1, PD-1 or a combination thereof. In some examples, the therapeutic composition is a pharmaceutical composition, containing at least one agent described herein together with a pharmaceutically acceptable carrier. In one embodiment, the composition includes a combination of multiple (e.g., two or more) agents described herein. In some embodiments, the combination of the first and second agent are administered in separate pharmaceutical compositions.

In some examples, the composition comprises an agent that disrupts the PD-L2/RGMb interaction, e.g., an antibody. Exemplary antibodies can be found in patents and published applications such as: U.S. Pat. Nos. 9,845,356, 10,370,448, US Patent Publication 2018/0002422, WO Pat. Publication WO2002000730, and US Patent Publication 2018/0258171, hereby incorporated by reference in their entireties.

In some examples, the composition comprises an agent that disrupts PD-L1 or PD-1, e.g., cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, CX-188, atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, or LY3415244.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

As described in detail below, the pharmaceutical compositions and/or agents disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

C. Kits

Described herein, in another aspect is a kit for treating a cancer in an individual comprising: a) a first agent that blocks or disrupts PD-L2, RGMb, or a combination thereof; b) a second agent that disrupts PD-L1, PD-1 or a combination thereof; and c) instructions for use of the first agent and the second agent in treating a cancer in an individual. In some examples, the kit is for the treatment of any of the indications disclosed herein. In some examples, a kit disclosed herein comprises the first agent and a second agent in amounts effective for use in a combination therapy, and a pharmaceutically acceptable carrier. In some examples, the first agent is any of the agents disclosed herein. In some examples, the first agent is an anti-PD-L2 antibody or an anti-RGMB antibody. In some examples, the second agent is any of the agents disclosed herein. In some examples, the second agent is an anti-PD1 antibody or an anti-PD-L2 antibody. In some examples, the first agent is disposed in a single container with the second agent. In some examples, the first agent is disposed in a first container, and the second agent is disposed in a second container. In some examples, the first agent and the second agent are to be administered approximately contemporaneously. In some examples, the first agent and the second agent are to be administered at different times.

In some examples, the kit comprises an agent that disrupts the PD-L2/RGMb interaction, e.g., an antibody. Exemplary antibodies are disclosed herein and can be found in patents and published applications such as: U.S. Pat. Nos. 9,845,356, 10,370,448, US Patent Publication 2018/0002422, WO Pat. Publication WO2002000730, and US Patent Publication 2018/0258171, hereby incorporated by reference in their entireties.

In some examples, the composition comprises an agent that disrupts PD-L1 or PD-1, e.g., cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, CX-188, atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, or LY3415244.

In some examples, a kit disclosed herein comprises an anti-PD-L2 antibody, a synergistically effective amount of an anti-PD-1 antibody or anti-PD-L1 antibody, and a pharmaceutically acceptable carrier or excipient. In some examples, a kit disclosed herein comprises an anti-RGMb antibody, a synergistically effective amount of an anti-PD-1 antibody or anti-PD-L1 antibody, and a pharmaceutically acceptable carrier or excipient.

### I. EXAMPLES

The following examples are included for illustrative purposes only and are not intended to limit the scope of the invention.

### Example 1: Mice Treated with Broad Spectrum Antibiotics have Dysbiosis

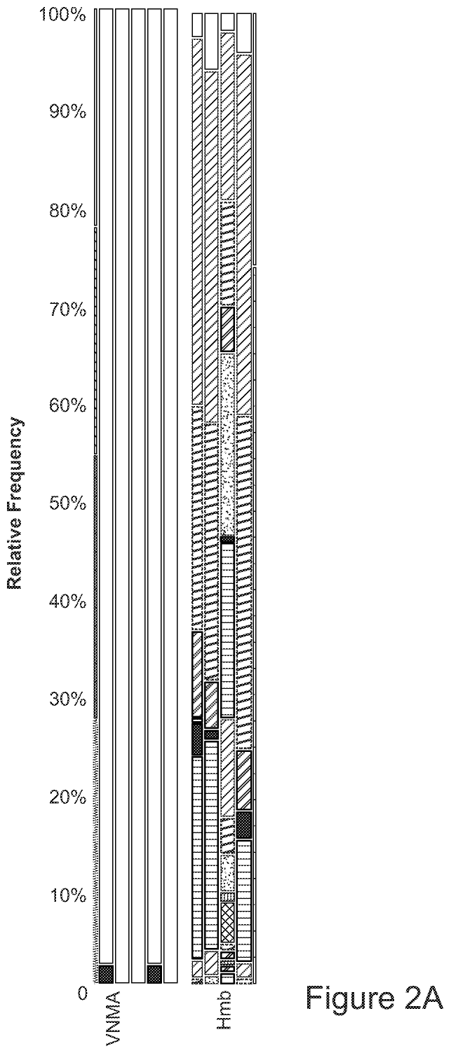

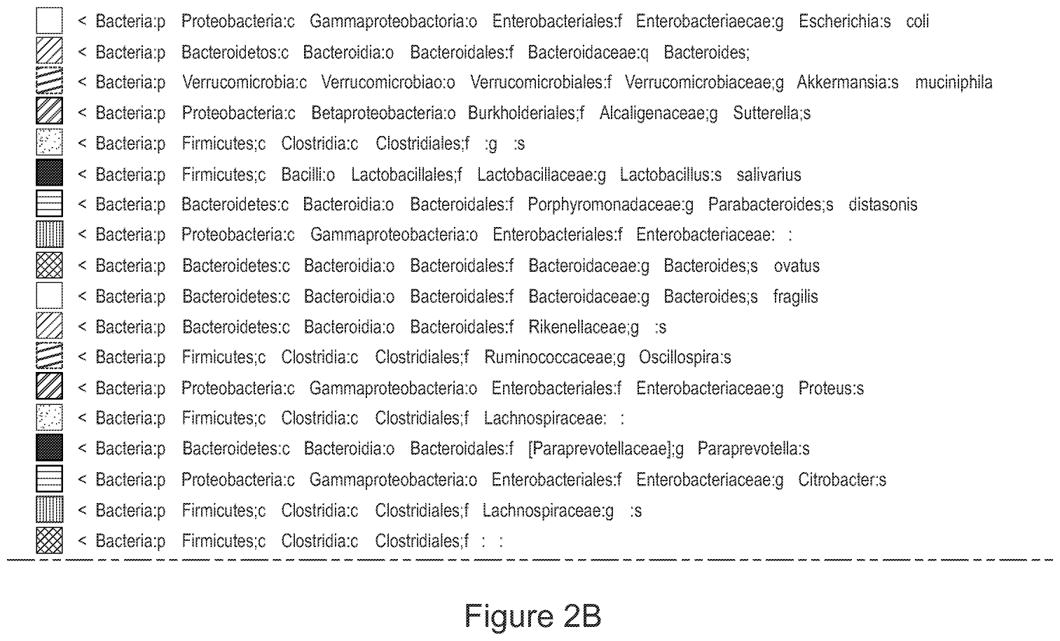

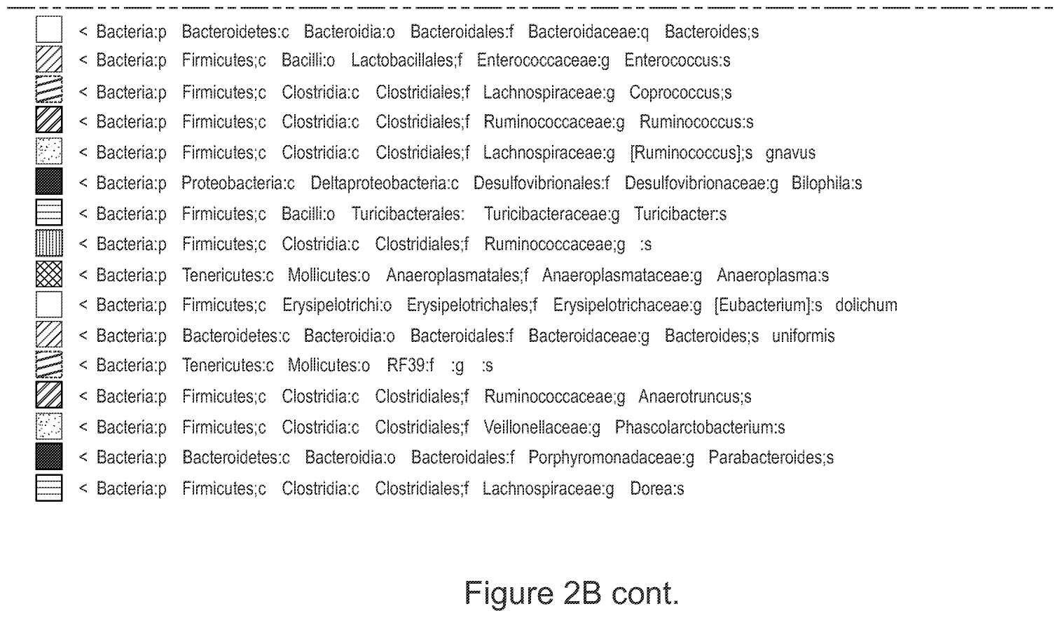

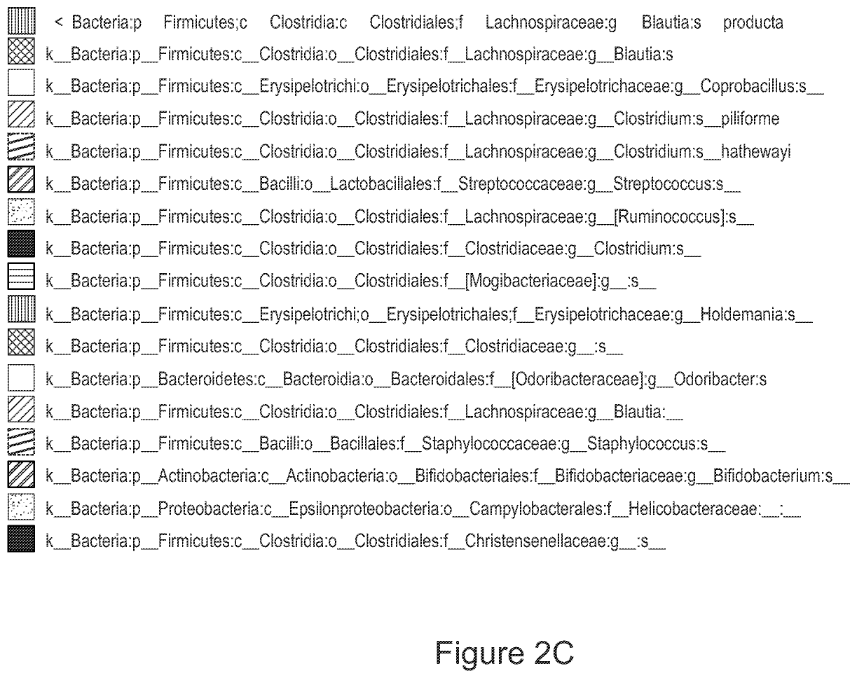

FIGS. 2-5 are data plots showing that mice treated with broad spectrum antibiotics (Vancomycin, Neomycin, Metronidazole, and Ampicillin or “VNMA/ABX”) have dysbiosis, an unhealthy microbiota.

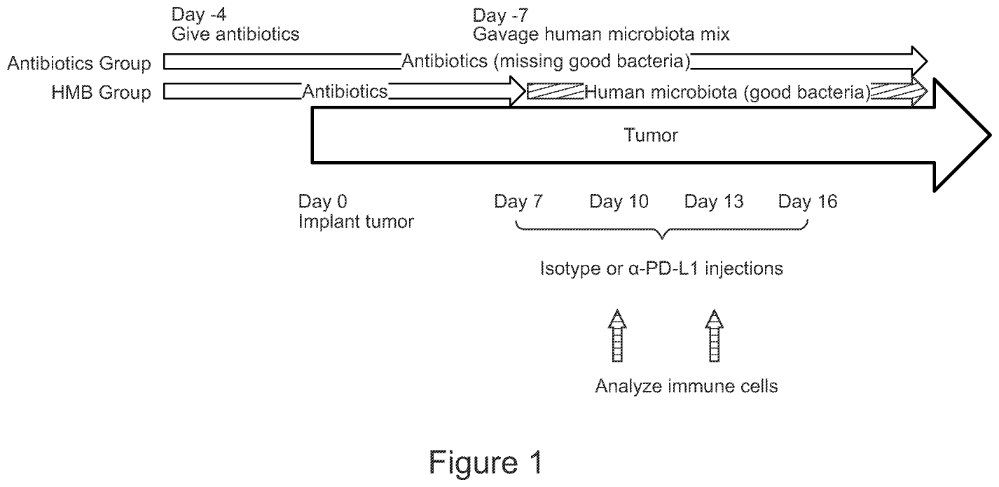

FIG. 1 shows the experimental timeline for FIGS. 1-4. Mice were given antibiotics (0.5 mg/ml Vancomycin, 1 mg/ml Neomycin, 1 mg/ml metronidazole, 1 mg/ml Ampicillin) in drinking water 4 days before tumor implantation. On day zero, 2.5×10⁵ MC38 tumor cells were implanted subcutaneously in the abdomen of 6 week old female mice. On days 7, 10, 13, 16 mice were treated with 100 μg of Isotype or anti-PD-L1 by intraperitoneal injection. On day 7, half of the mice were orally gavaged with a slurry of Hmb feces and antibiotics were removed from the drinking water; the other half of the mice did not receive Hmb feces and continue with antibiotics in the drinking water for the remainder of the experiment. Tumors were measured on days 7, 10, 13, 16, 20, 23.

FIG. 2 shows that mice treated with broad spectrum antibiotics (VNMA/ABX) for 17 days have an altered microbiota, referred to as dysbiosis. This microbiota is made up dominantly of a Proteobacteria, E. coli. Mice given a dose of healthy human microbiota (Hmb) have a much more diverse microbiota.

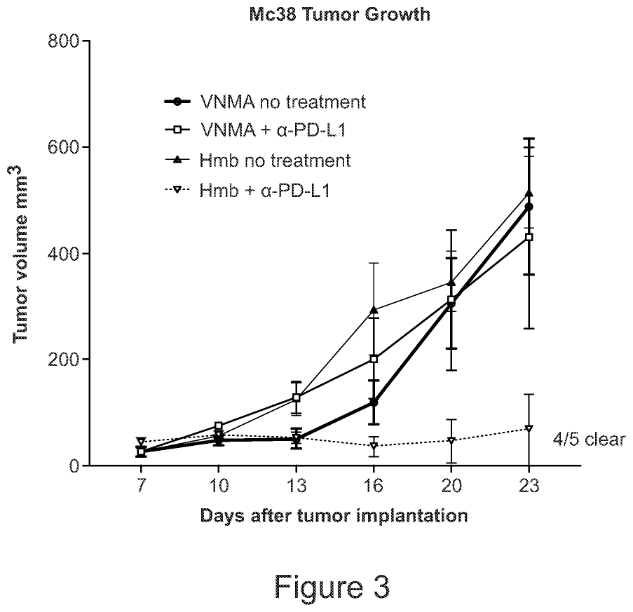

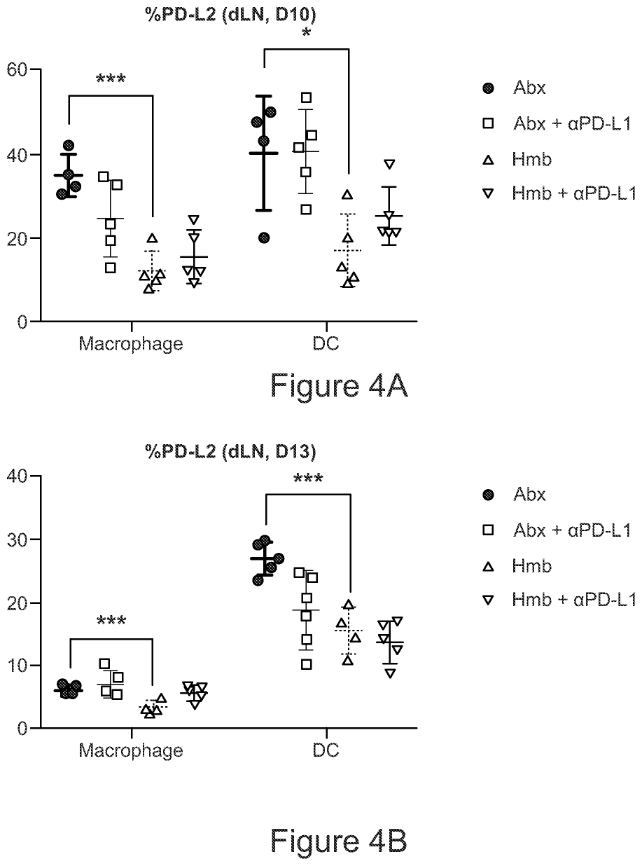

FIG. 3 shows that mice given VNMA do not respond to anti-PD-L1 therapy whereas mice given an oral dose of Hmb can clear or have significantly smaller tumors indicating that dysbiosis inhibits the anti-tumor effect of anti-PD-L1 therapy. To determine how dysbiosis abrogates the anti-tumor effects of anti-PD-L1 therapy, the expression of surface markers on the immune cells in the tumor draining lymph nodes of VNMA versus Hmb mice were compared. It was found that PD-L2 expression was significantly lower in macrophages and dendritic cells of Hmb mice, but higher in VNMA mice which were less response to anti PD-L1 therapy (FIG. 4).

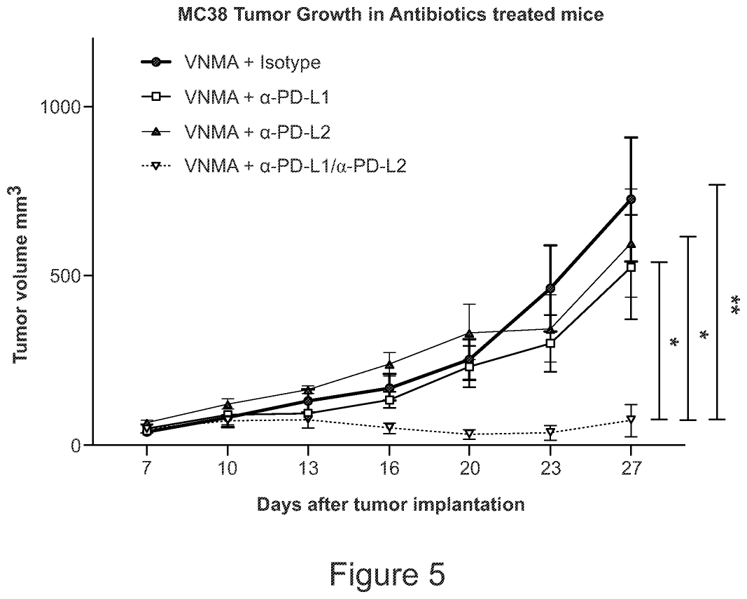

To determine if disrupting PD-L2 in VNMA mice would reverse the effects of dysbiosis on anti-tumor immunity, anti-PD-L1 and anti PD-L2 were combined in VNMA treated mice. FIG. 5 shows that while anti-PD-L1 and anti-PD-L2 individually do not promote an anti-tumor response in VNMA treated mice, they synergistically promote an anti-tumor response. These data show that combined anti-PD-L1 and anti-PD-L2 therapy can reverse the immune-suppressive effects of dysbiosis and promote anti-tumor responses. These data imply that in the clinic, patients with associated dysbiosis and poor response to anti-PD-L1 treatment, could benefit from combined anti-PD-L1 and anti-PD-L2 therapy.

### Example 2: Effects of Anti-PD-L1/Anti-PD-L2 Therapy in Germ Free Mice

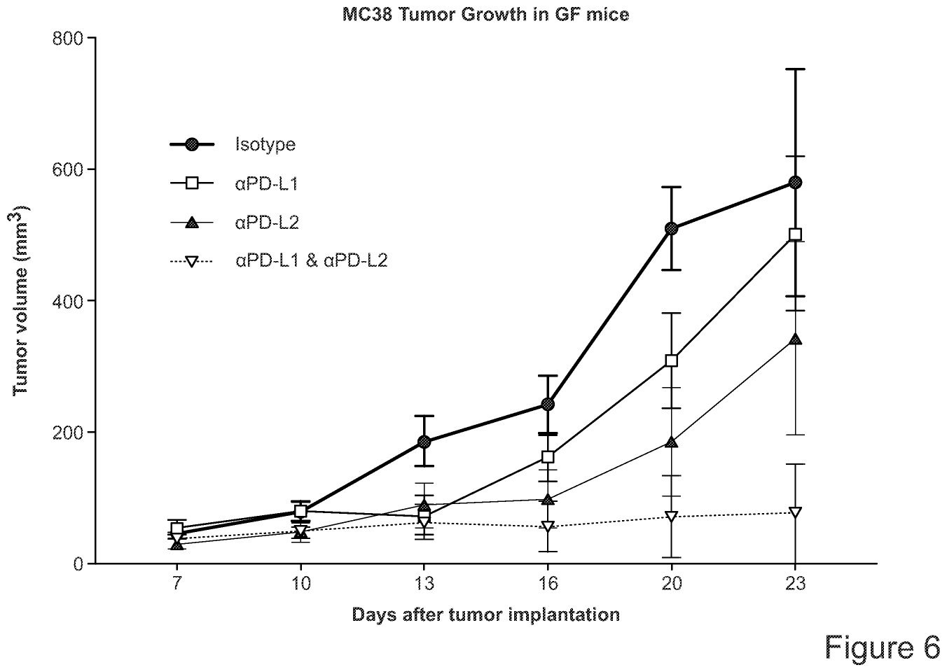

Germ free mice were implanted with MC38 cells, colon carcinoma, and treated with four doses of 100 μg of a control, an anti-PD-L1 antibody (clone: 10F.9G2), an anti-PD-L2 antibody (clone: 3.2 or GF17.2C9) disrupting PD-L2/RGMb interaction without disrupting the interaction between PD-L2 and PD-1, or an anti-PD-L1/anti-PD-L2 antibody combination every three day, starting on the post-implantation day 7. Tumor volume measured over the course of 23 days. See FIG. 6. As can be seen, combined anti-PD-L1 and anti-PD-L2 therapy, but not anti-PD-L1 or anti-PD-L2 therapy alone, promotes an anti-tumor response in germ free mice implanted with a highly immunogenic tumor. These data suggest that targeting PD-L2 mediated signaling pathways including the PD-L2/RGMb pathway is important for anti-tumor responses in patients receiving anti-PD-1 therapy.

### Example 3: Effects of Anti-PD-1/Anti-PD-L2 Therapy in Germ Free Mice

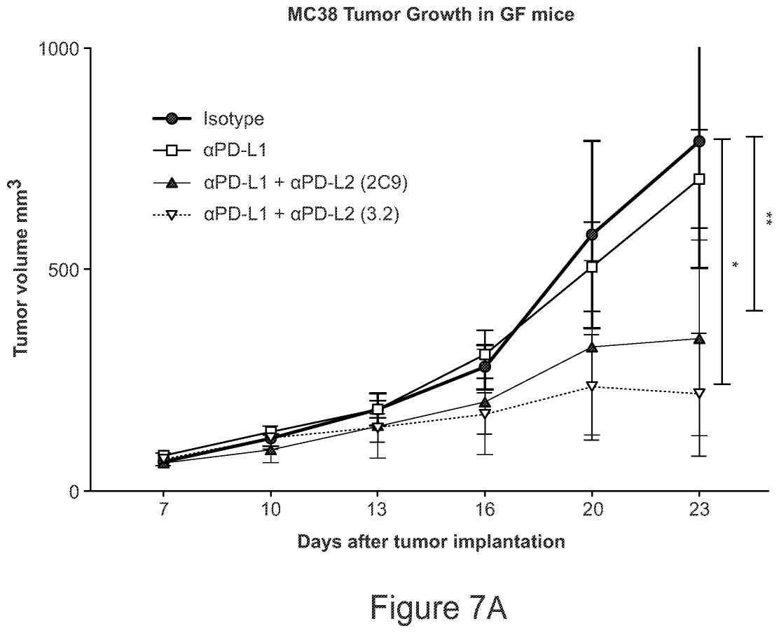

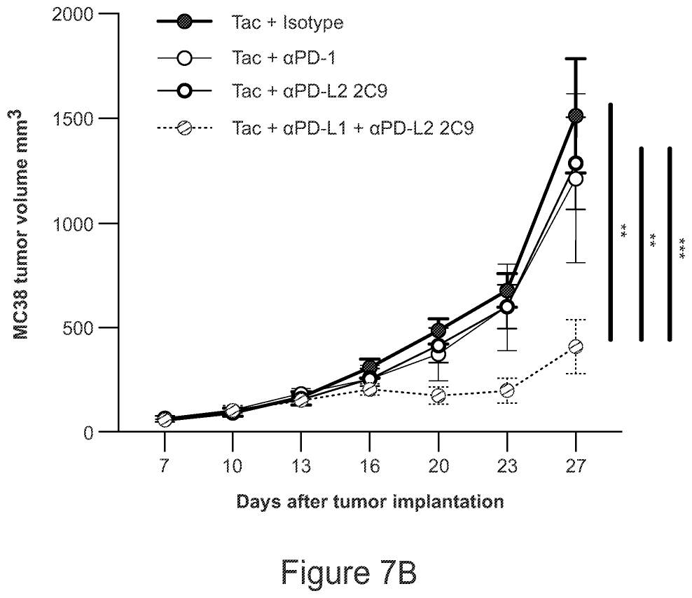

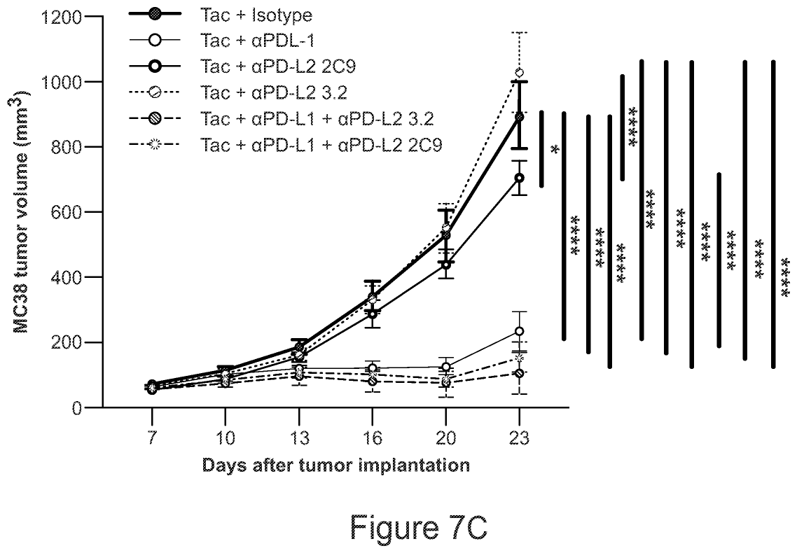

Germ free mice were implanted with MC38 cells (colon carcinoma) and treated with four doses of 100 μg of a control, an anti-PD-1 antibody (clone: RMP1-14) or an anti-PD-L2 antibody (clone: 3.2 or GF17.2C9) combination every three day, starting on the post-implantation day 7. Mouse anti-PD-L2 antibodies, clones GF17.2C9 (blocking PD-L2/RGMb interaction) and 3.2 (blocking PD-L2/PD-1 interaction) were used. Tumor volume measured over the course of 23 days. See FIG. 7A. FIG. 7 shows that combined anti-PD-1 and anti-PD-L2 therapy, but not anti-PD-1 therapy, promotes an anti-tumor response in germ free mice. Of note, both αPD-L2 antibodies (blocking PD-L2/PD-1 and PD-L2/RGMb interactions) showed a synergism with αPD-1 treatment in both GF and specific pathogen free mice (from Taconic Biosciences, Inc., “Tac”) whilst αPD-1 monotherapy did not promote an anti-tumor response. See FIG. 7A, B. These data suggest that targeting PD-L2 mediated signaling pathways including the PD-L2/RGMb pathway is important for anti-tumor responses in patients receiving anti-PD-1 therapy. To further test the translational potential of αPD-L2 antibodies to patients with a complex microbiota, we used Tac mice, in which the microbiota was shown to induce a lesser degree of response to αPD-L1 treatment. As expected, combinatorial treatment of αPD-L1 and αPD-L2 antibodies or αPD-L1 alone strongly suppressed growth of the highly immunogenic MC38 tumors (FIG. 7C).

### Example 4: Effects of Anti-PD-1 or Anti-PD-L1/Anti-RGMB Therapy in Germ Free Mice

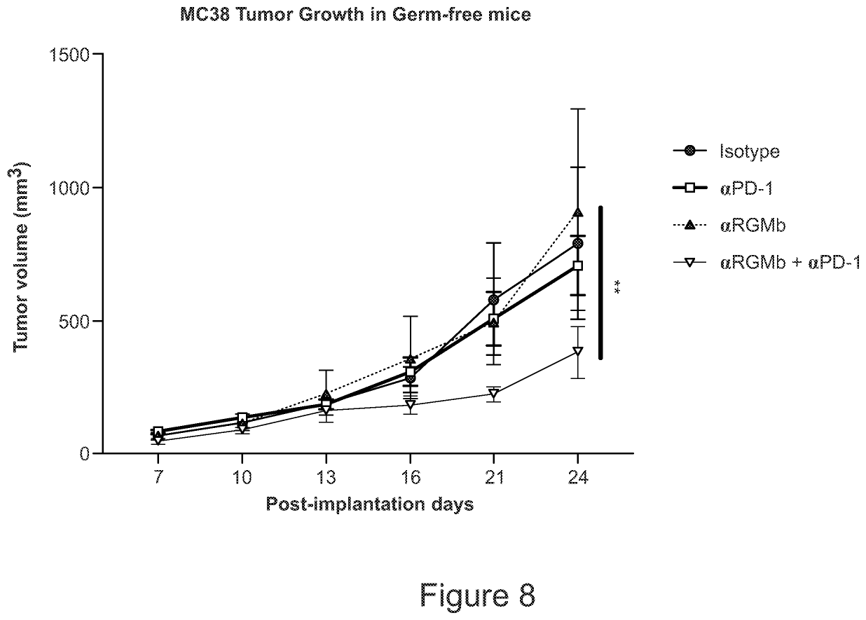

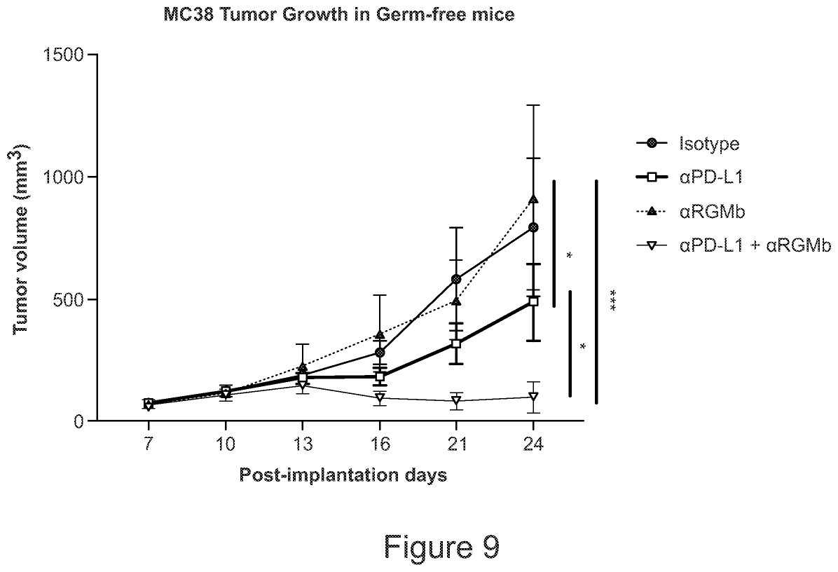

Because anti-PD-L2 mAb clone GF17. 2C9 (2C9), which disrupts the interaction between PD-L2 and RGMb without disrupting the interaction between PD-L2 and PD-1, promotes an anti-tumor response in GF mice with either anti-PD-L1 or anti-PD-1 treatment, we tested whether targeting RGMb instead of PD-L2 could also promote an anti-tumor response in GF mice treated with either anti-PD-1 or anti-PD-L1. Germ free mice were implanted with MC38 cells (colon carcinoma) and treated with four doses of 100 μg of a control, an anti-PD-1 antibody (clone: RMP1-14), an anti-RGMB antibody (clone: 307.9D1) or an anti-PD-1/anti-RGMB antibody combination every three day, starting on the post-implantation day 7. In another data set, germ free mice were implanted with MC38 cells (colon carcinoma) and treated with a control, an anti-PD-L1 antibody, an anti-RGMB antibody or an anti-PD-L1/anti-RGMB antibody combination. Tumor volume measured over the course of 23 days. See FIGS. 8 and 9. It was found that combination anti-RGMb and either anti-PD-1 (FIG. 8) or anti-PD-L1 (FIG. 9) promotes a significant anti-tumor response in GF mice suggesting that disrupting the PD-L2/RGMb response by not only targeting PD-L2 but also targeting RGMb can promote an anti-tumor response in patients receiving anti-PD-1 or anti-PD-L1 therapy.

### Example 5: PD-L2 and RGMb Transcription is Upregulated in Lymph Node Dendritic Cells from Antibiotic Treated Mice

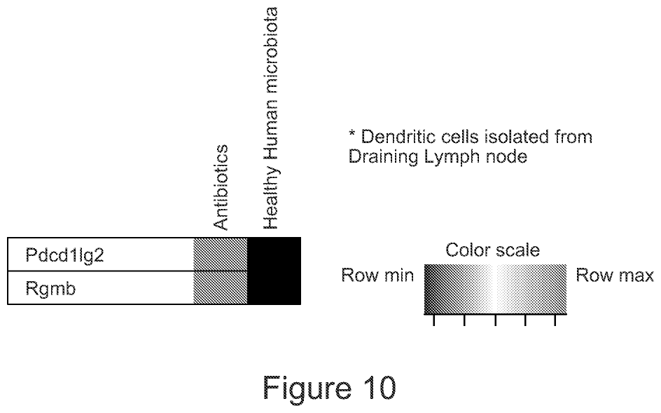

Two groups of mice were treated with broad spectrum antibiotics in the drinking water 4 days before tumor implantation. In one group of mice (HMB), antibiotic treatment was stopped seven days after tumor implantation and a healthy human microbiota was transplanted into the mice. In the other group (antibiotics), mice received antibiotics in the drinking water for the entire experiment. The expression of PD-L2 and RGMb in the cells isolated from both the antibiotic-treated mice and healthy human microbiota mice were determined using techniques known in the art. Briefly, dendritic cells were collected by flow cytometry into a lysis buffer then flash frozen by dry-ice and stored at −80° C. Smart-Seq2 libraries for low-input RNA-seq were prepared by the Broad Technology Labs and were subsequently sequenced through the Broad Genomics Platform. Normalized gene expression was analyzed using MultiPlot. FIG. 10 provides a gene expression heat map of comparing the expression of PD-L2 and RGMb in the cells isolated from the antibiotic treated mice versus the mice having a healthy human microbiota. As can be seen from FIG. 10, PD-L2 (Pdcd1lg2) and Rgmb transcription was increased in dendritic cells isolated from tumor draining lymph nodes in antibiotic treated mice as compared to the mice with a healthy human microbiota.

### Example 6: Effects of Anti-PD-L1/Anti-PD-L2 Therapy in VMNA Mice

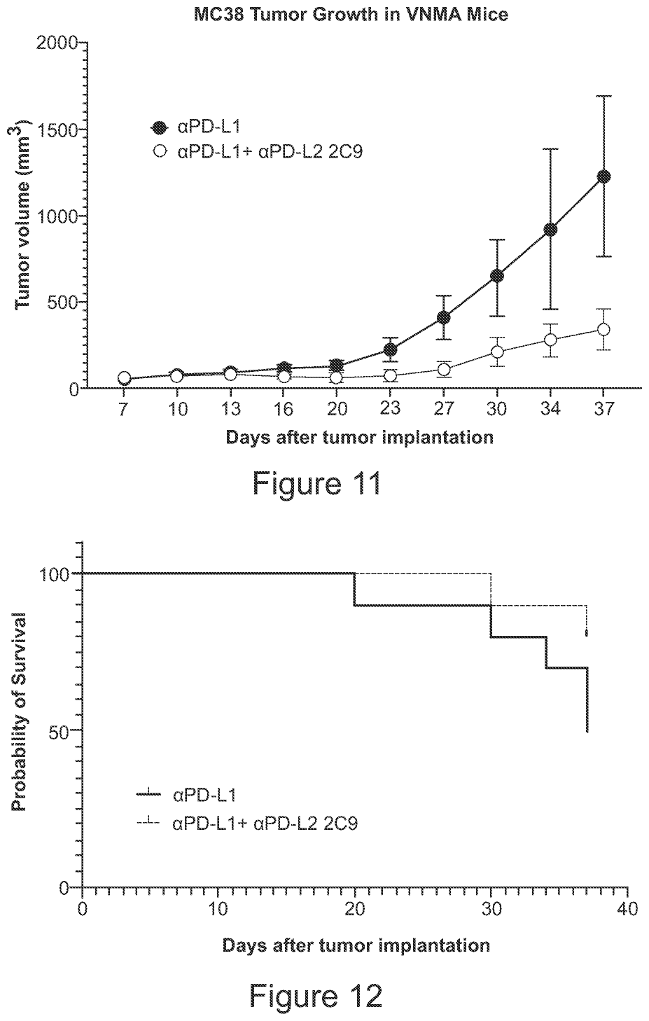

The effects of anti-PD-L1 therapy versus anti-PD-L1/anti-PD-L2 therapy on antibiotic treated mice (dysbiotic) implanted with MC38 tumors were analyzed. Mice were given antibiotics (0.5 mg/ml Vancomycin, 1 mg/ml Neomycin, 1 mg/ml metronidazole, 1 mg/ml Ampicillin) in drinking water 4 days before being implanted with M38 cells. On day zero, MC38 tumor cells are implanted subcutaneously in the abdomen of 6 week old female mice. On days 7, 10, 13, 16 mice (10 per group) are treated with an anti-PD-L1 antibody or a combination of anti-PD-L1 and anti-PD-L2 (2C9) antibodies. Tumors were measured daily, and tumor volume over time produced by each treatment is depicted in FIG. 11 (days 7, 10, 13, 16, 20, 23, 27, 30, 34 and 37), which shows that combined anti-PD-L1 and anti-PD-L2 therapy promotes a more durable anti-tumor response in antibiotic treated mice than anti-PD-L1 therapy alone. The probability of survival of the anti-PD-L1 treated versus anti-PD-L1/anti-PD-L2 treated mice over time is shown in FIG. 12. As can be seen in FIG. 12, anti-PD-L2 therapy combined with anti-PD-L1 therapy increases survival compared to anti-PD-L1 therapy alone.

### Example 7: Effects of Anti-PD-L1/Anti-PD-L2 Therapy in Dysbiotic Mice Implanted with B16-OVA

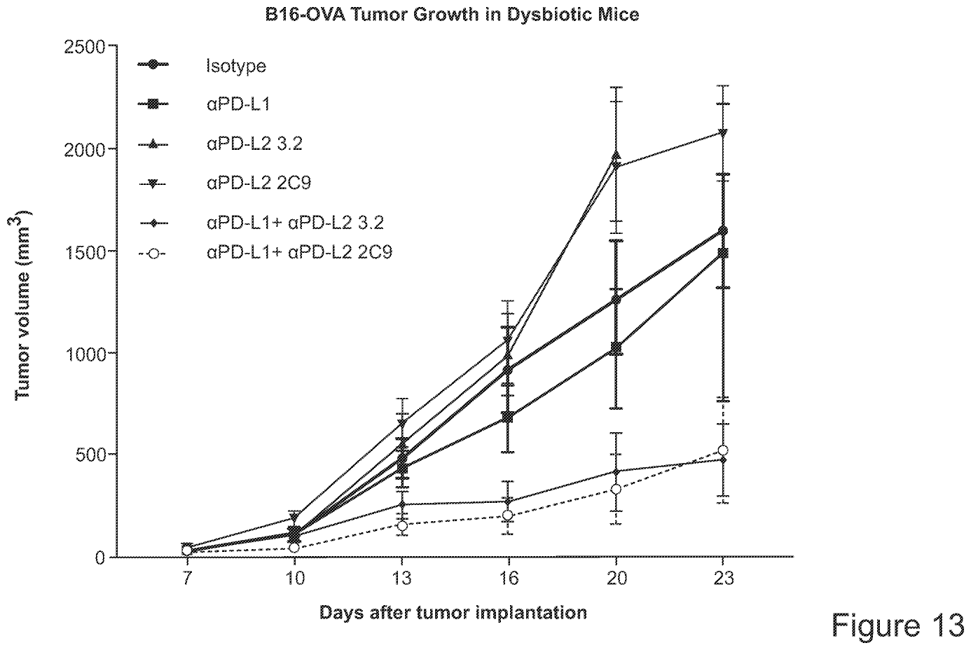

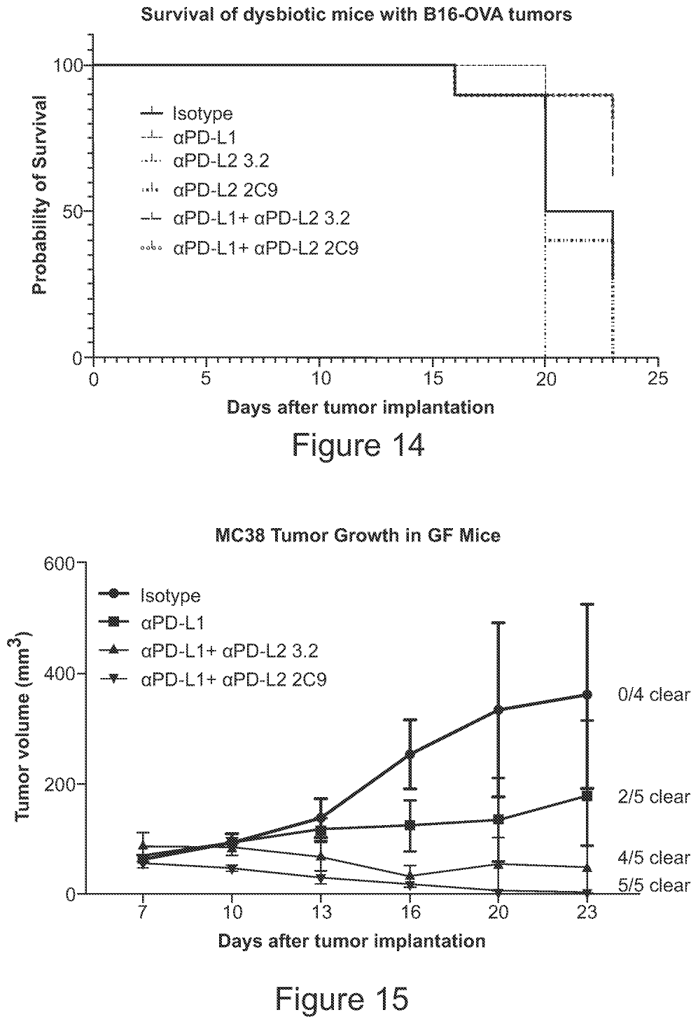

The effects of anti-PD-L1 therapy versus anti-PD-L2 versus anti-PD-L1/anti-PD-L2 therapy on antibiotic treated mice (dysbiotic) implanted with B-16-OVA immunogenic tumors were analyzed. Mice were given antibiotics (0.5 mg/ml Vancomycin, 1 mg/ml Neomycin, 1 mg/ml metronidazole, 1 mg/ml Ampicillin) in drinking water 4 days before being implanted with B160VA cells. On day zero, B16-OVA tumor cells (murine melanoma) were implanted subcutaneously in the abdomen of 6 week old female mice. On days 7, 10, 13, 16 mice (10 per group) were treated with either an anti-PD-L1 antibody or a combination of anti-PD-L1 and anti-PD-L2 (2C9 or 3.2) antibodies. Tumors were measured daily and days 7, 10, 13, 16, 20, and 23 are shown. The tumor volume over time resulting from each treatment is depicted in FIG. 13, which shows that combined anti-PD-L1 and anti-PD-L2 therapy, but not anti-PD-L1 therapy alone, promotes an anti-tumor response in dysbiotic mice implanted with a tumor that is more resistant to immunotherapy. The probability of survival of the anti-PD-L1 treated versus anti-PD-L1/anti-PD-L2 treated mice over time is shown in FIG. 14. As can be seen in FIG. 14, anti-PD-L2 therapy combined with anti-PD-L1 therapy increases survival compared to anti-PD-L1 therapy alone.

### Example 8: Effects of Anti-PD-L1/Anti-PD-L2 Therapy in Germ Free Mice

Germ free mice were implanted with MC38 cells (colon carcinoma) and treated with four doses of 100 μg of a control, an anti-PD-1 antibody (clone: RMP1-14) or an anti-PD-1/anti-PD-L2 antibody (clone: 3.2 or GF17.2C9 blocking PD-L2/RGMb interaction and 3.2 blocking PD-L2/PD-1 interaction) combination every three day, starting on the post-implantation day 7. Tumor volume was measured over the course of 23 days. See FIG. 15. FIG. 15 shows that combined anti-PD-L1 and anti-PD-L2 therapy, but not anti-PD-L1 therapy, promotes an anti-tumor response in germ free mice. These data suggest that targeting PD-L2 mediated signaling pathways including the PD-L2/RGMb pathway is important for anti-tumor responses in patients receiving anti-PD-L1 therapy.

### Example 9: Impact of Microbiota on Co-Inhibitory Molecule Gene Expression

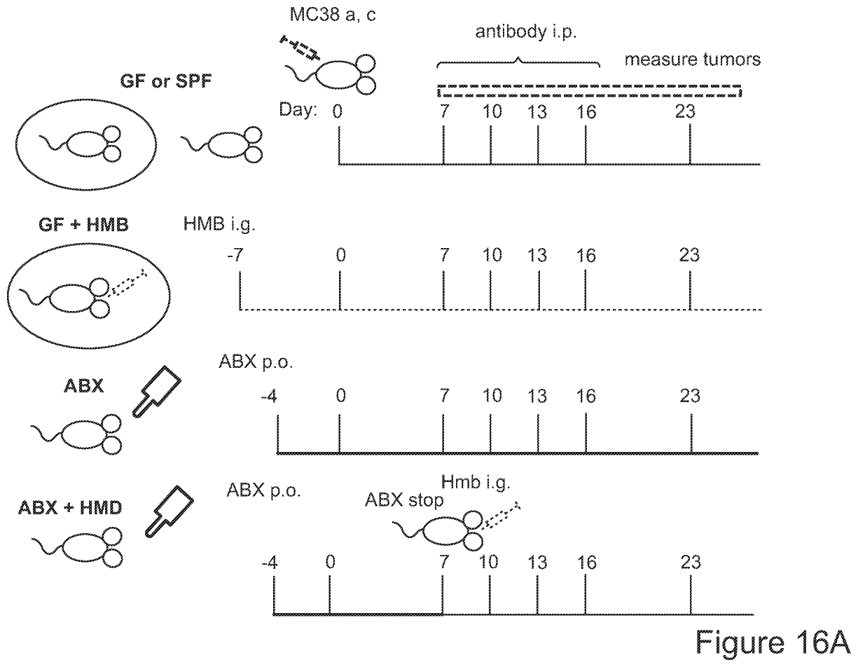

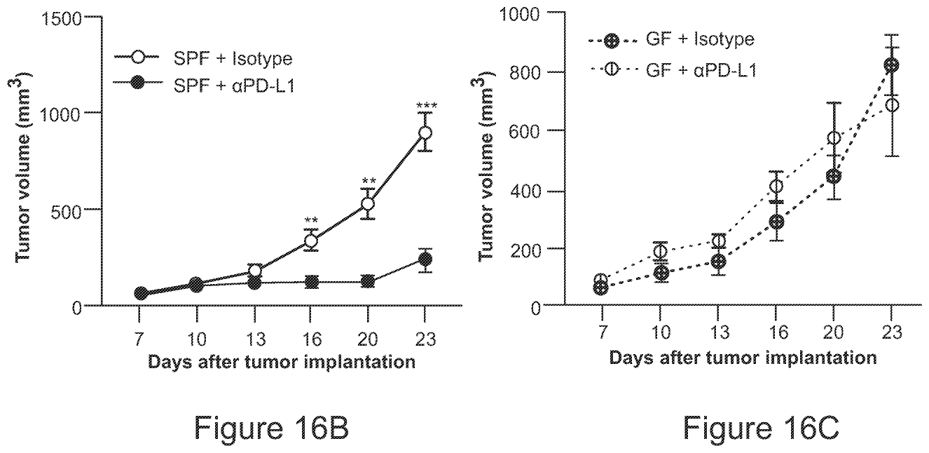

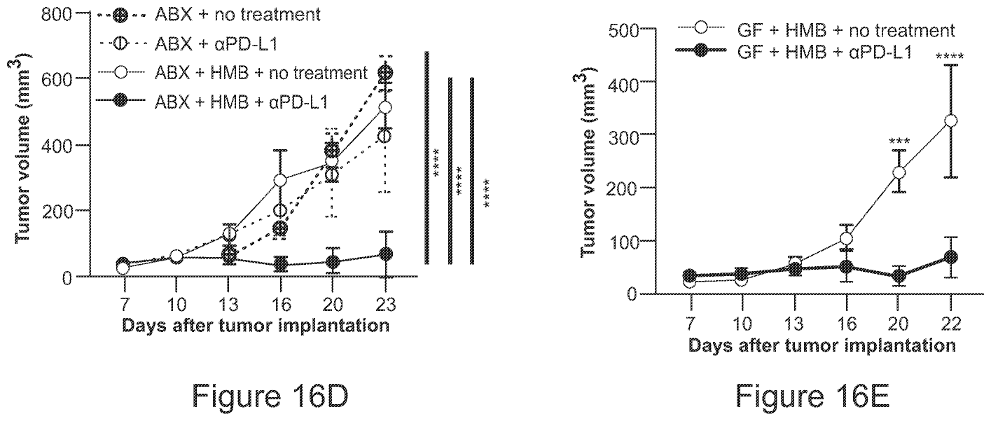

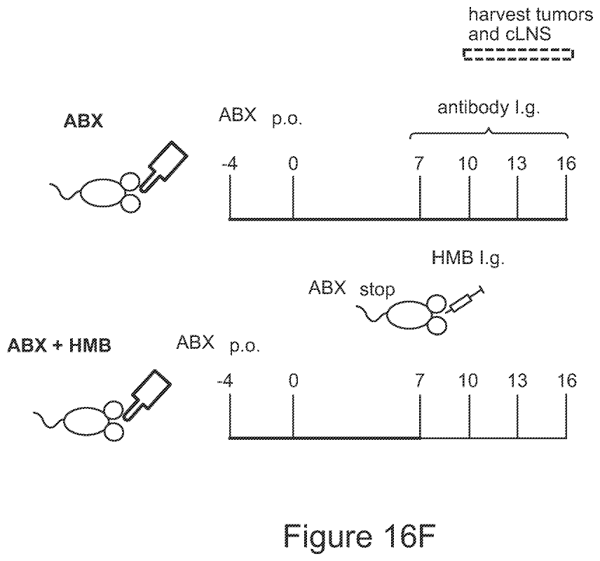

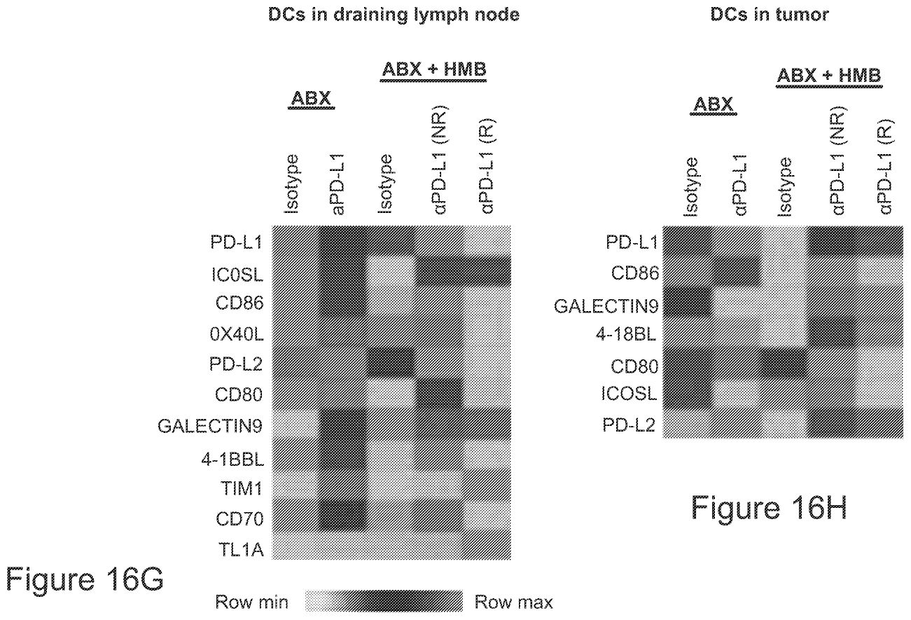

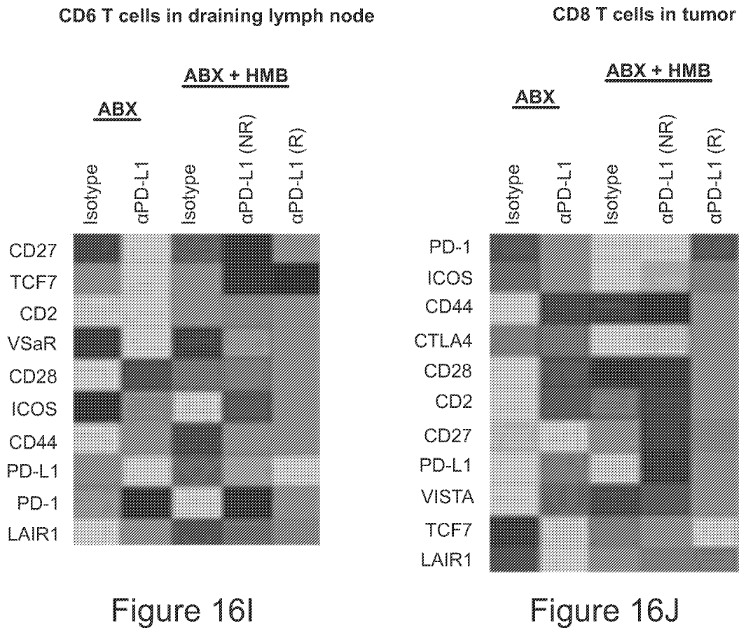

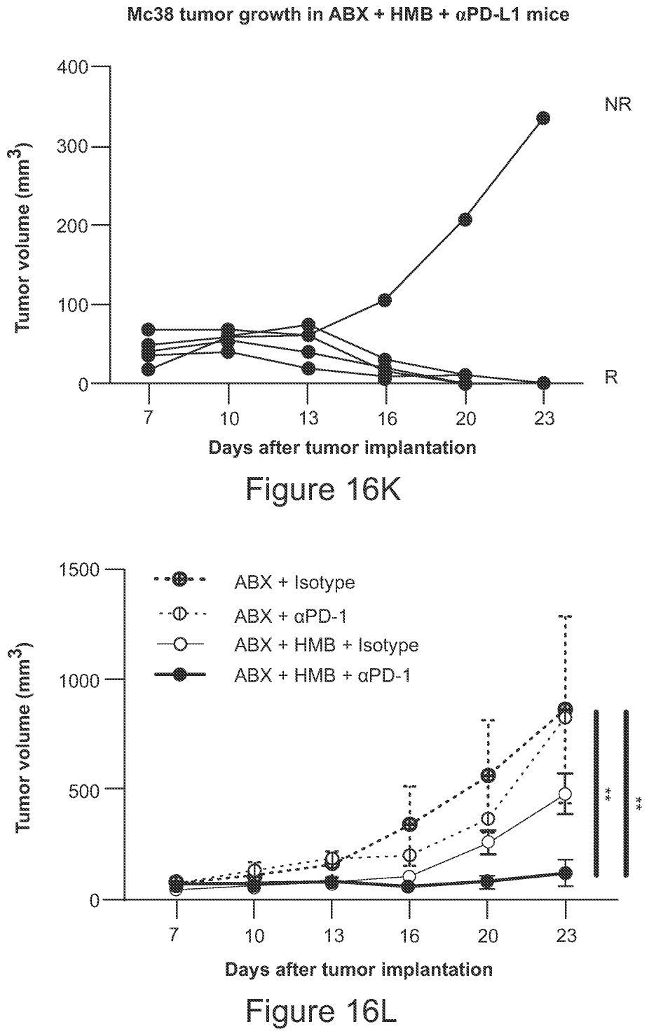

To investigate the role of gut microbiota in regulating responses to immune checkpoint inhibitors, mouse tumor models utilizing germ-free (GF) mice or mice treated with a combination of broad-spectrum antibiotics (Vancomycin, Neomycin, Ampicillin, and Metronidazole; ABX) were established (FIG. 16A). Conventional specific pathogen free (SPF; Taconic Biosciences, Inc.) mouse response to αPD-L1 therapy was confirmed (FIG. 16B) while absence or depletion of gut commensals prevented αPD-L1 or αPD-1 induced regression of MC38 colon carcinomas in mice (FIG. 16C, D). Additionally, inoculating GF or ABX mice with the well-characterized healthy human gut microbiota (HMB) promoted an anti-tumor response to αPD-L1 (FIG. 16D, E) or αPD-1 (FIG. 16K). The clear difference between ABX and ABX+HMB provided an excellent system to further examine molecular mechanisms by which the gut microbiota regulates anti-immune responses during αPD-L1 treatment (FIG. 16D). Transcriptomes of immune cells from tumors and tumor draining lymph nodes (dLNs) were analyzed at post-implantation (p.i.) day 16, when distinction of responders and non-responders started to appear (FIG. 16F, L). Notably, expressions of key T cell co-stimulatory and co-inhibitory molecules in CD11c⁺MHC class II^{hi} dendritic cells (DC) and CD8⁺ T cells were largely modulated by gut microbiota and anti-tumor response (FIG. 16G-J).

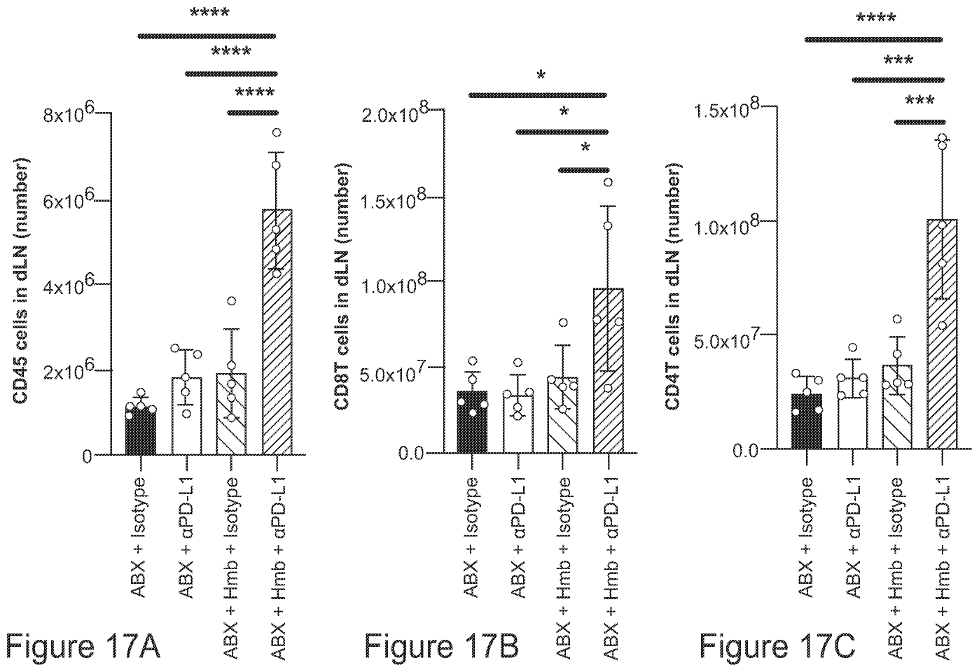

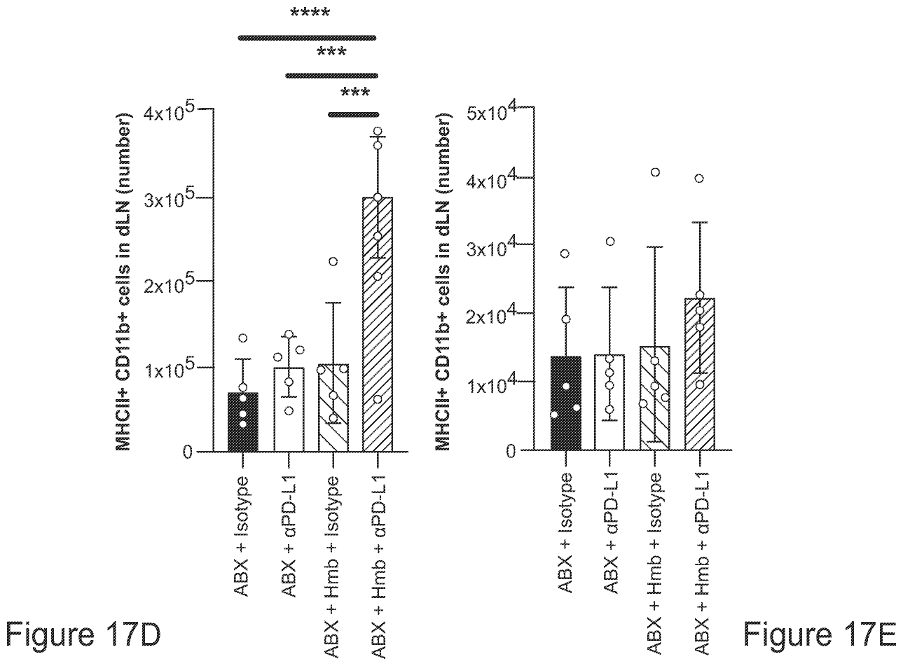

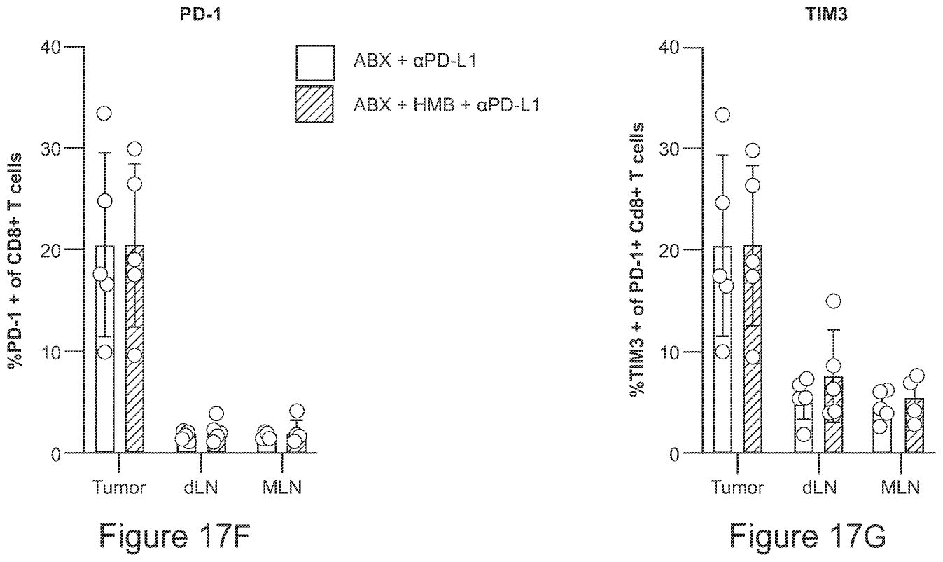

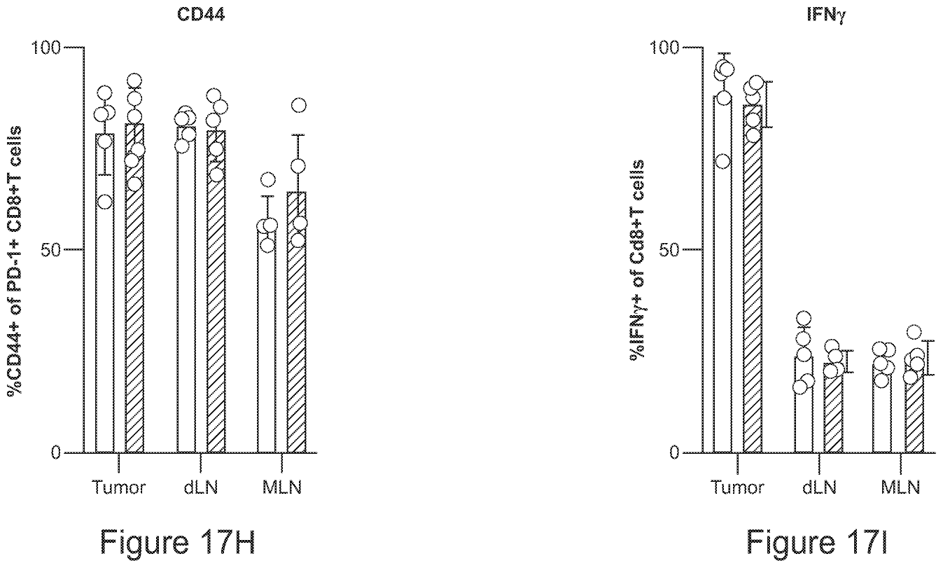

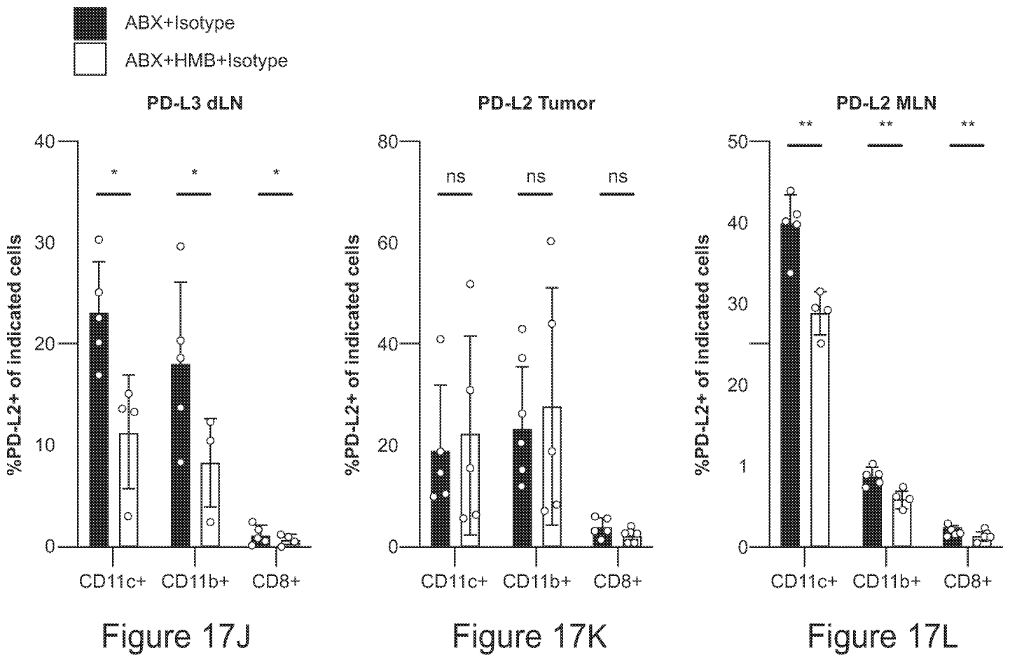

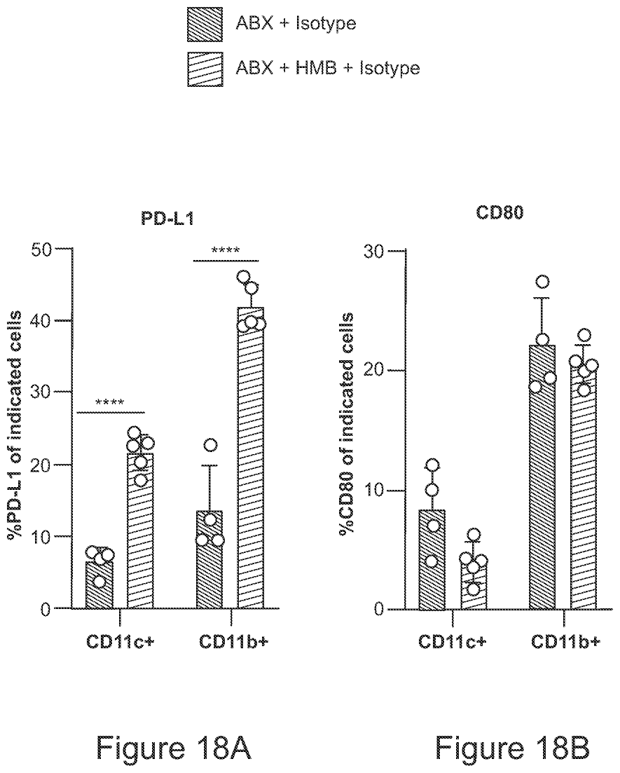

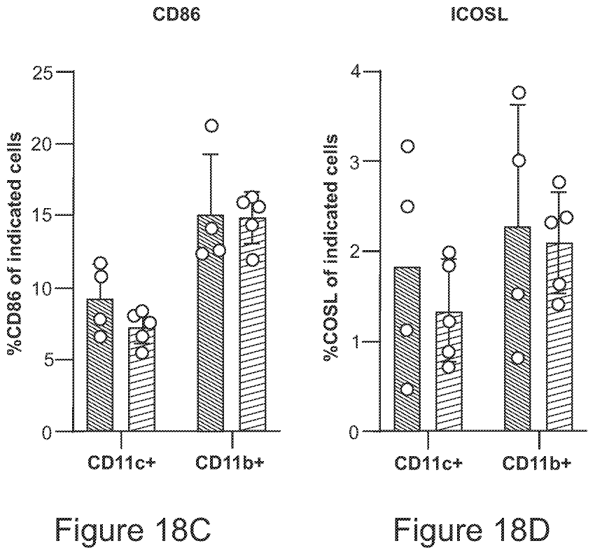

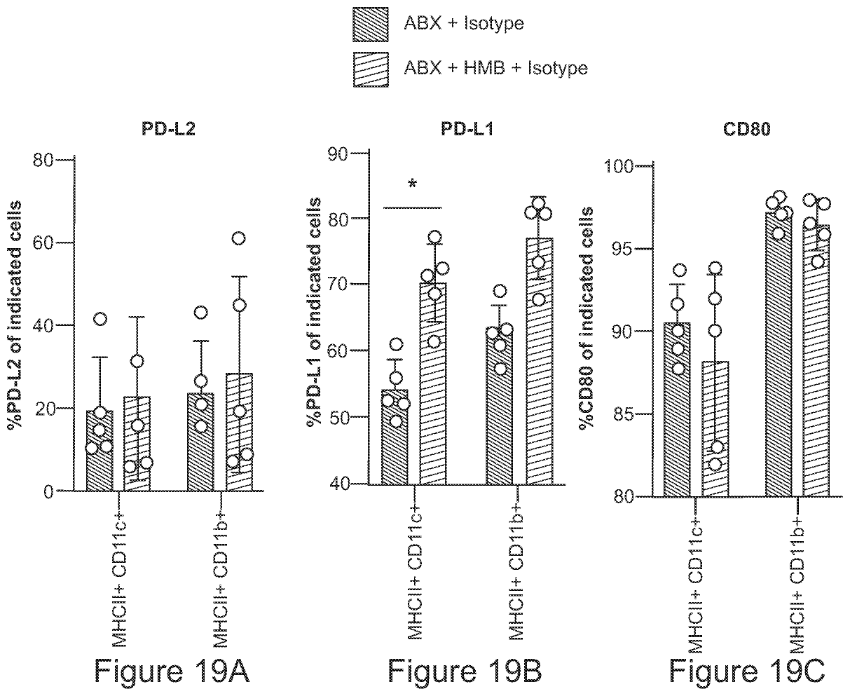

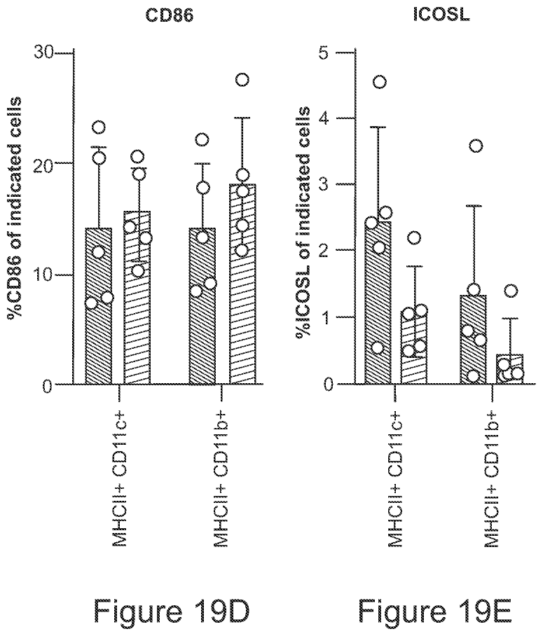

To identify which co-stimulatory and co-inhibitory molecules played a causative role in an anti-tumor response, immune cells from the dLNs and tumors at days 10-13 were analyzed, before tumor sizes significantly diverged in (i) isotype versus (ii) αPD-L1 treated mice. A change in cell count of a specific cell type prior the exhibition of a significant divergence in tumor size between isotype versus αPD-L1 treated mice was thought to suggest that molecules promoting anti-tumor response mediated by said specific cell type were molecules which played a causative role in anti-tumor response. At post-implantation day 13, the numbers of CD45⁺ immune cells, CD8⁺ cells, CD4⁺ T cells, and MHCII⁺ CD11b⁺ cells, but not MHCII⁺ CD11c⁺ cells in the dLNs were synergistically increased by HMB and αPD-L1 treatment, compared to ABX or Isotype groups (FIG. 17A-E). However, CD8⁺ T cell expression of PD-1, PD-1⁺ TIM3⁺ (exhausted), CD44^{+ PD-}1⁺ (activated), or IFNγ⁺, which has previously been shown to be increased in the microbiota mediated response to PD-1 blockade at later timepoints, was not significantly different in ABX vs ABX+HMB mice treated with αPD-L1 (FIG. 17F-I) indicating that capture of the timepoint before checkpoint blockade significantly enabled CD8⁺ T cell anti-tumor function in responder (ABX+HMB+αPD-L1) mice. To determine which co-stimulatory and co-inhibitory molecules on dendritic cells might have a causative role in promoting a CD8⁺ T cell mediated anti-tumor response, protein expression of several co-signaling molecules that were identified in RNAseq data to be impacted by the microbiota were measured. Notably, PD-L2, and PD-L1 were differentially expressed on CD11b⁺MHC class II⁺ and CD11c⁺MHC class II⁺ cells in the dLNs of ABX vs ABX+HMB mice while CD80, CD86, and ICOSL showed no significant differences (FIG. 17J, FIG. 18A-D). Tumor antigen presenting cells only showed significant differences in expression of PD-L1 between ABX vs ABX+HMB mice (FIG. 19A-E). Collectively, the data suggest that the gut microbiota regulates specific signaling pathways—particularly PD-L2 signaling pathways—involved in T cell co-stimulation.

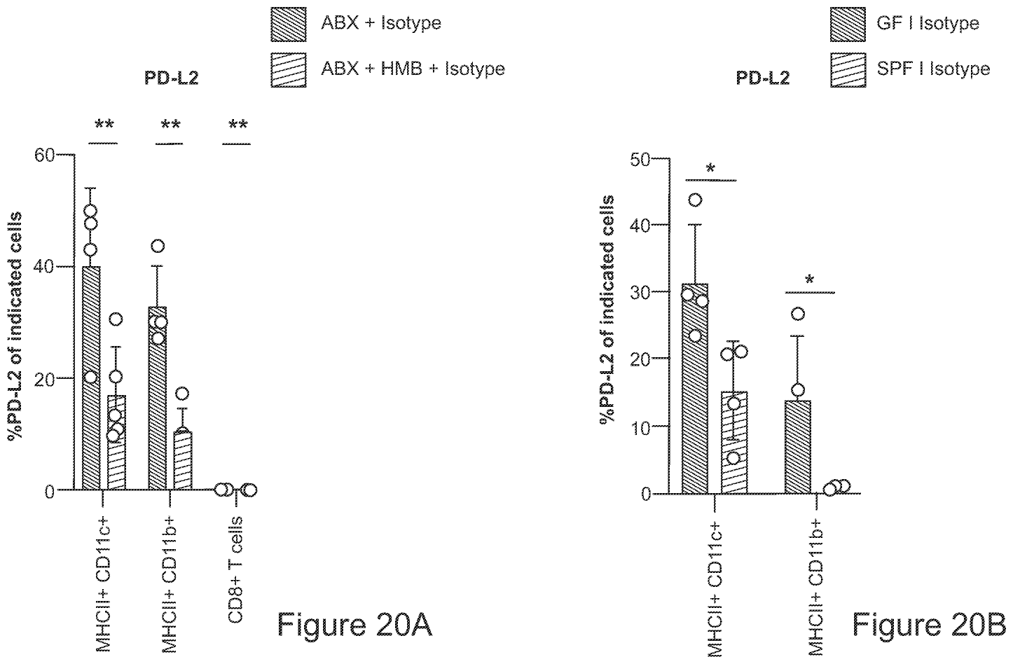

In identifying a specific pathway that played a causal role between gut microbiota and αPD-L1-mediated anti-tumor immunity, it was noted that PD-L2, a co-inhibitory molecule, was suppressed on CD11c⁺ and CD11b⁺ subsets in ABX+HMB compared to ABX mice at day 13 post implantation (p.i.) in dLNs and mesenteric lymph nodes (MLNs), but not in tumors. (FIG. 17J-L). This change was observed even at an earlier time point (day 10 p.i.) in the dLNs of ABX vs ABX+HMB and GF vs Specific-Pathogen-Free (SPF) mice (FIG. 20A, B). Given that dLNs play an important role in controlling anti-tumor immunity induced by PD-L1 blockade, suppression of systemic anti-tumor immunity by upregulation of PD-L2 on antigen presenting cells in ABX mice is consistent with the data presented herein, and suggests that PD-L2 serves a critical immunoregulatory role in the absence of the gut microbiota.

### Example 10: RGMb Regulates Anti-Tumor Immunity in GF Mice

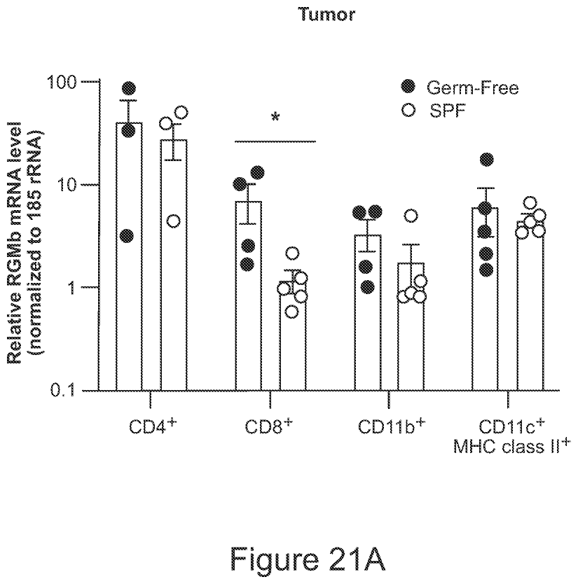

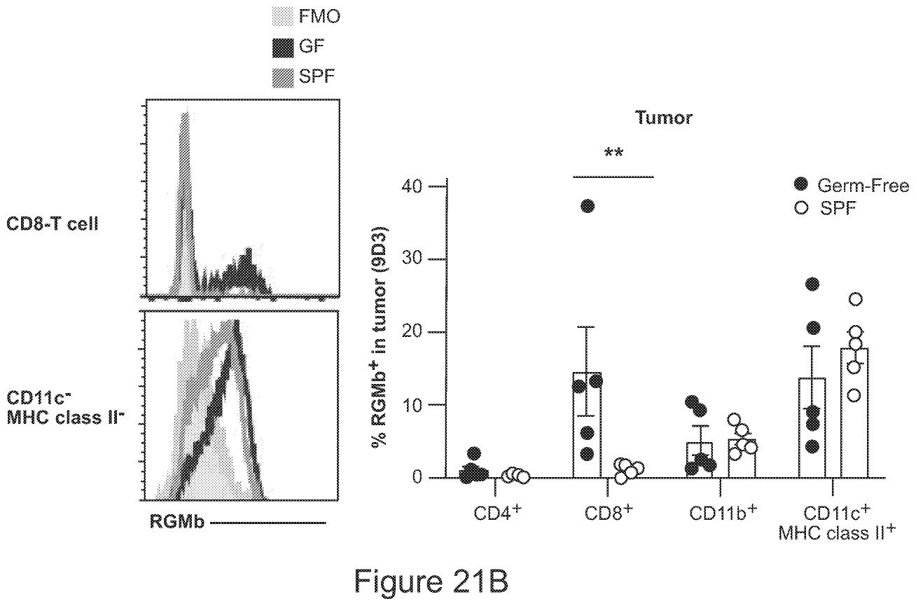

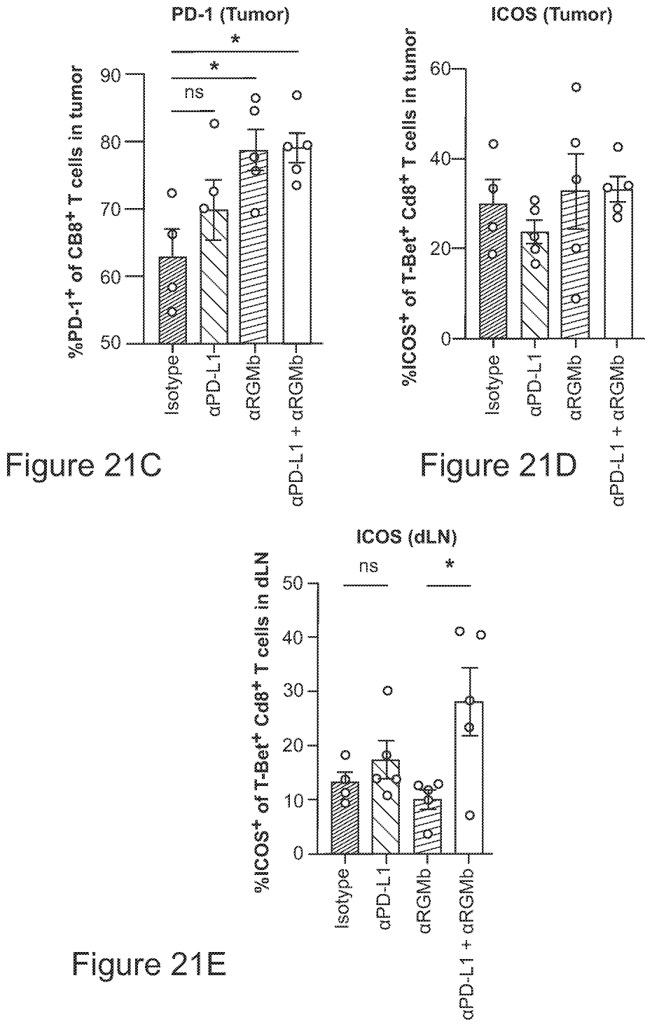

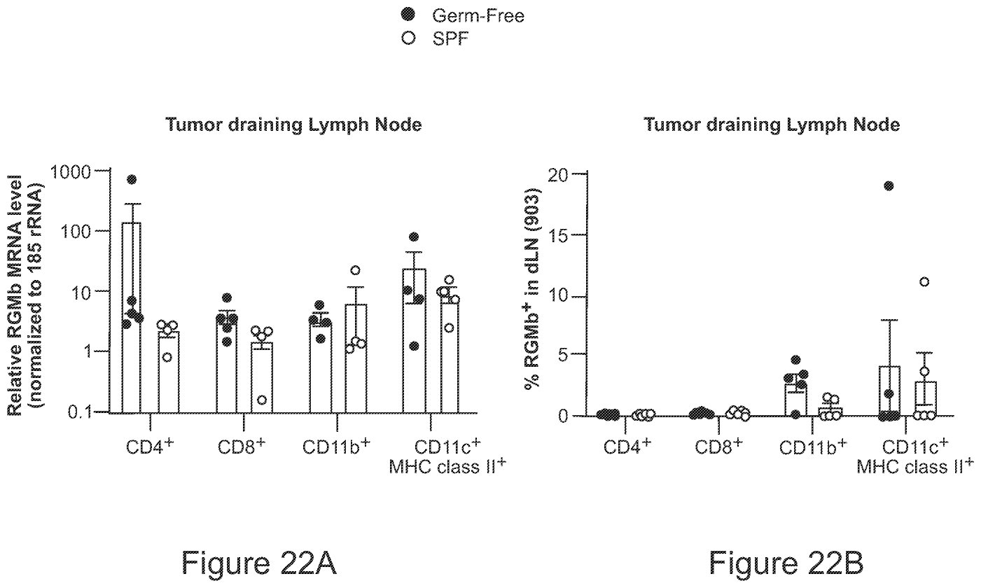

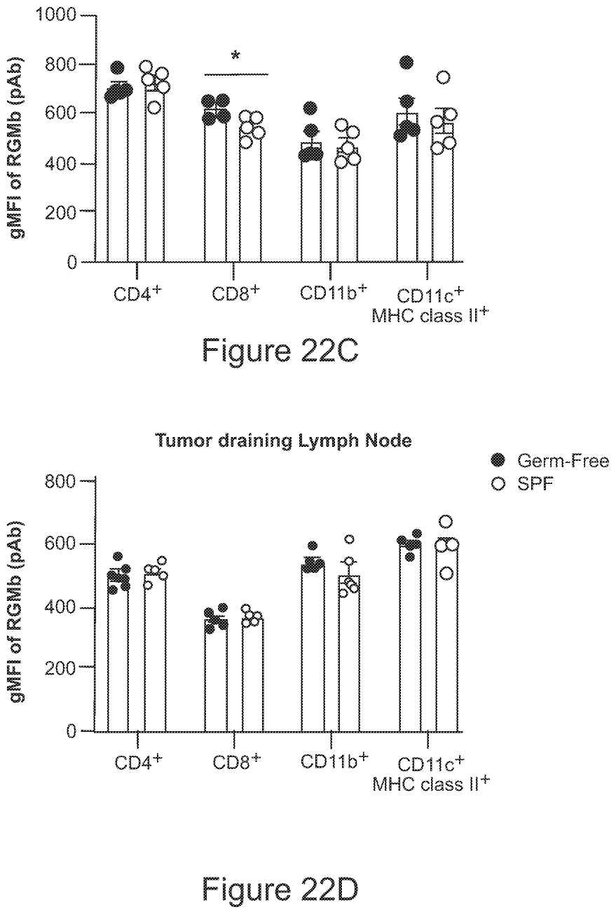

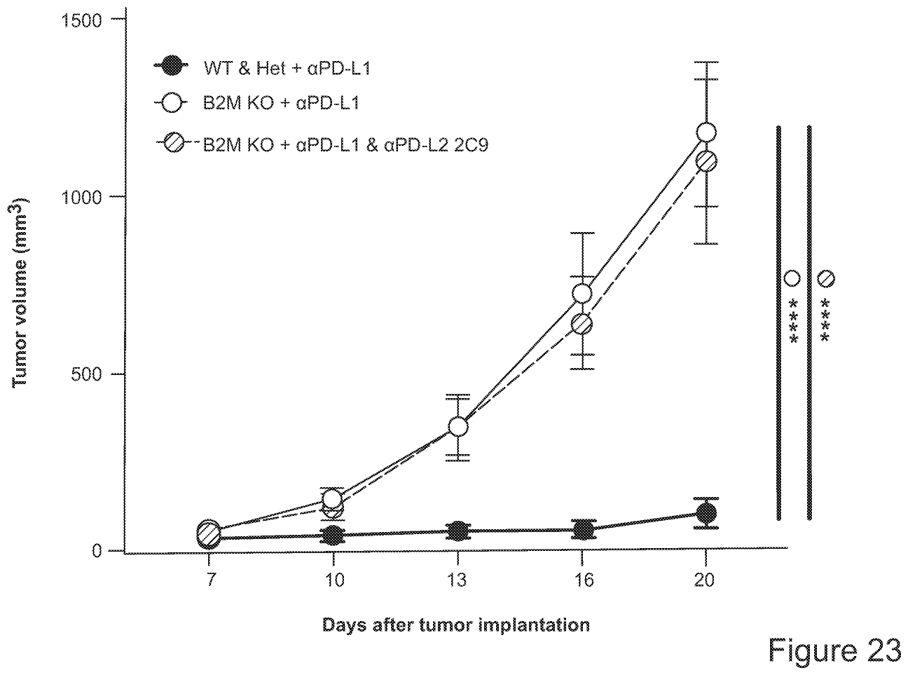

To obtain a mechanistic understanding of regulation of RGMb/PD-L2 by the gut microbiota, RGMb expression in SPF and GF mice was measured. Transcript levels of RGMb in CD8⁺ Tumor infiltrating T cells were 6.1-fold higher in GF mice compared to SPF mice (FIG. 21A). When surface expression levels of RGMb protein were measured by a monoclonal antibody (9D3 clone) or a polyclonal antibody (FIG. 21B, FIG. 22), CD8⁺ tumor-infiltrating T cells from GF mice expressed significantly higher levels of RGMb. Differences in RGMb expression in other cell subsets were not significant (FIG. 21B), indicating that RGMb expressed on T cells plays an important role in CD8⁺ T cell mediated anti-tumor immunity. In accordance with this finding, β2m−/− mice lacking CD8⁺ T cells fail to respond to combined treatment of αPD-L1 and 2C9 clone in the MC38 tumor model, confirming a functional relevance of CD8⁺ T cell and RGMb/PD-L2 pathway (FIG. 23). Taken together with the synergistic effect observed in αRGMb/αPD-L1 or αPD-1 treatment (i.e., FIGS. 8 and 9), these findings suggest that blocking the PD-1/PD-L1 pathway in combination with blocking the PD-L2/RGMb pathway can promote an anti-tumor response in patients who do not respond to PD-1/PD-L1 pathway blockade alone.

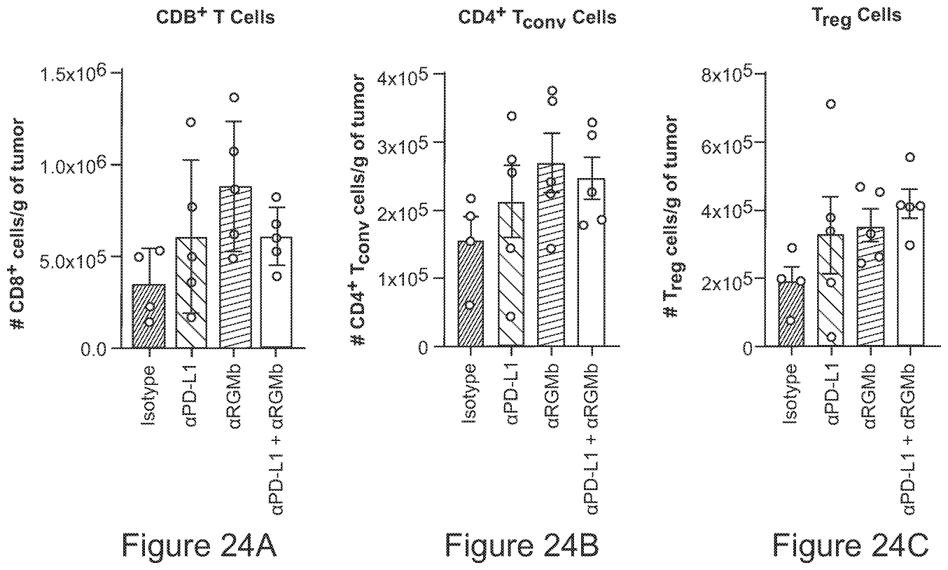

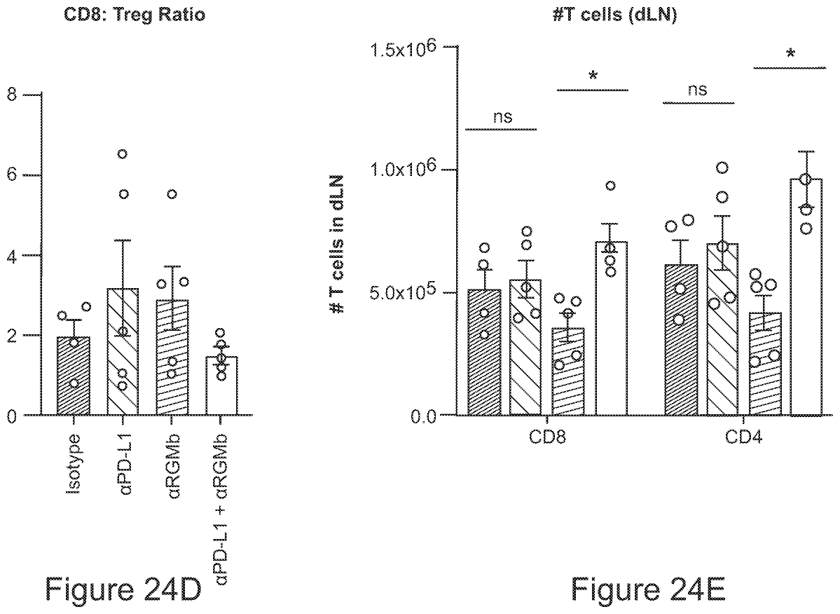

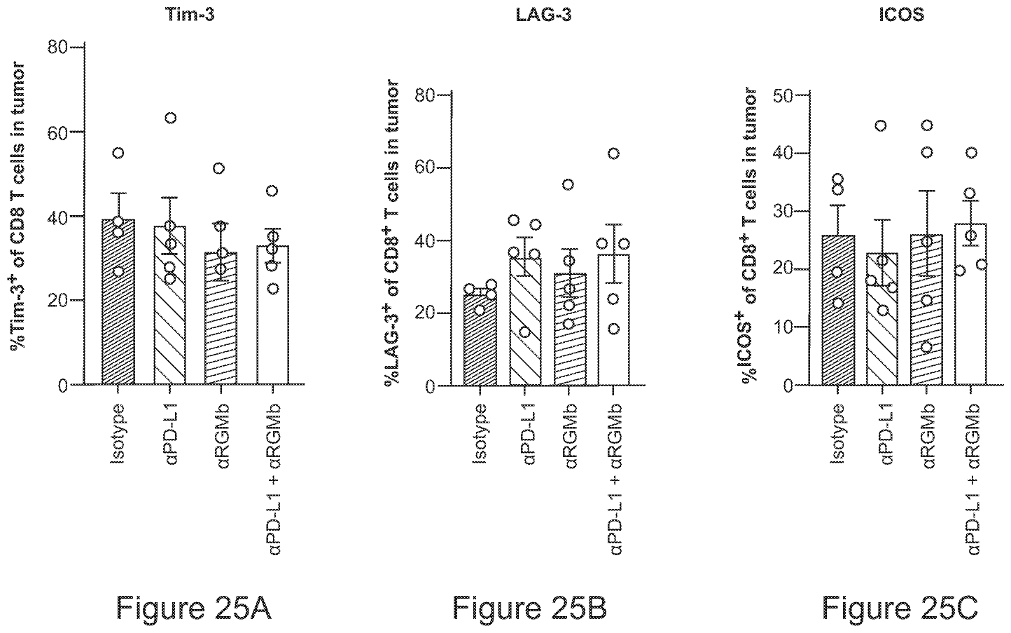

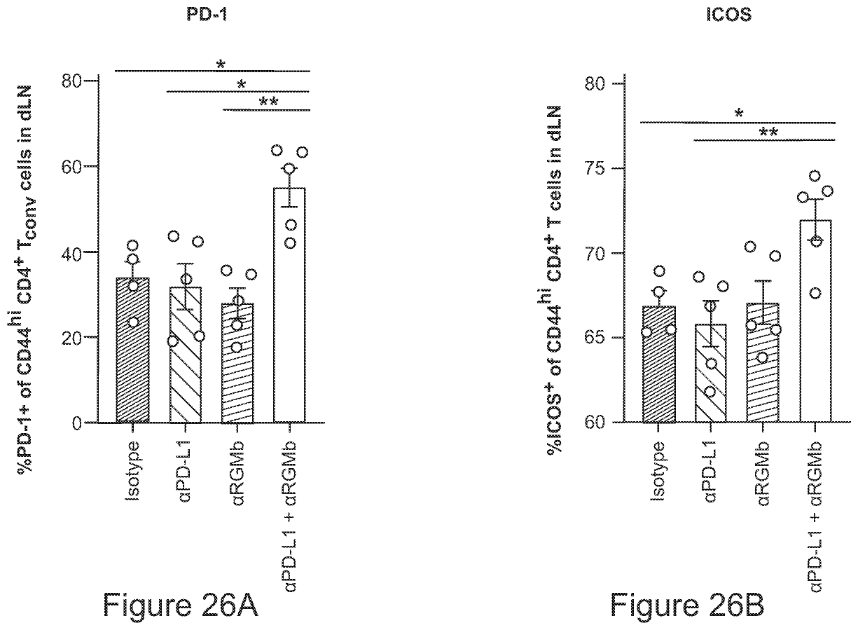

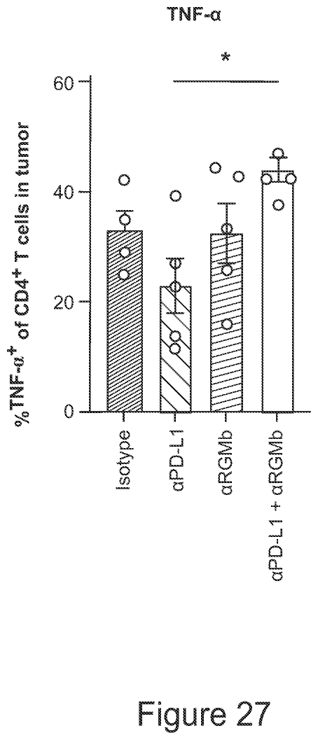

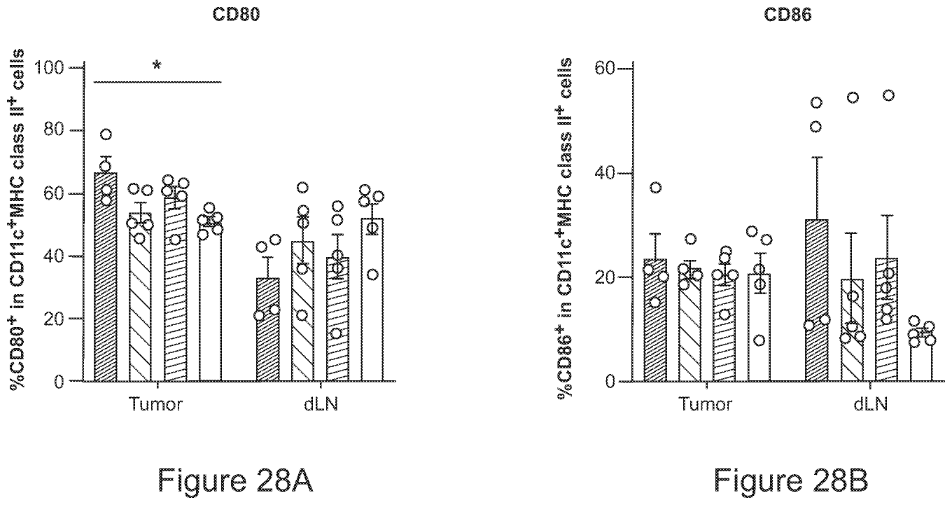

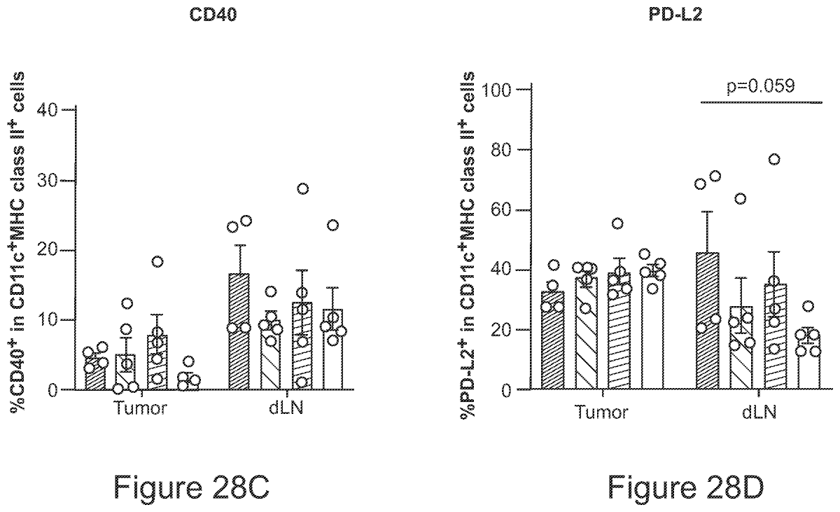

To characterize the change in immune profiles induced by αRGMb treatment in GF mice, immune cell subsets at p.i. day 11 were analyzed. Overall T cell numbers in tumors were not significantly changed by either αPD-L1 or αRGMb at this time point (FIG. 24). Notably, PD-1 expression on CD8⁺ tumor-infiltrating cells was increased in αRGMb treated groups without upregulation of other co-inhibitory molecules including TIM-3 and LAG-3 (FIG. 21C, FIG. 25). Since the ICOS pathway has been implicated in positively regulating the efficacy of checkpoint blockade (16), the expression of ICOS in tumor and dLN after treatment of αPD-L1 and αRGMb was examined (FIG. 21D,E). ICOS expression on T-bet⁺ CD8⁺ T cells, which were previously shown to be increased in mice treated with anti-tumor gut bacteria and checkpoint inhibitors, was up-regulated by αPD-L1 and αRGMb treatment in the dLNs of GF mice (FIG. 21E), but not in the tumor (FIG. 21D). Of note, the CD4⁺ T cell compartment also exhibited changes in ICOS expression in dLN. αPD-L1 and αRGMb treatment led to elevated PD-1 and ICOS expression on CD4 T⁺ cells in a synergistic manner (FIG. 26), suggesting that CD4⁺ T cells might be also involved in anti-tumor immunity regulated by RGMb. In addition, Tumor Necrosis Factor γ (TNF-γ) production by CD4⁺ T cells was significantly increased by αRGMb treatment (FIG. 27), suggesting a potential role of RGMb in T cell mediated pro-inflammatory cytokine expression. Patterns of expression of co-stimulatory ligands such as CD80, CD86 and CD40 on CD11c⁺MHC class II⁺ cells did not show a notable or consistent difference between αRGMb and/or αPD-L1 treated groups (FIG. 28). Collectively, the data suggests that targeting RGMb during immune checkpoint inhibition might have a positive impact on T cell signaling capacity for potent T cell activation.

### Example 11: Clinical Trial Design for Anti-PD-1/Anti-RGMB Combination

Clinical evaluation of an anti-PD-1 and anti-RGMB combination therapy is conducted in patients suffering from head and neck, breast, endometrial, ovarian, and prostate or bladder cancers, with trials designed to confirm the efficacy and safety of the combination therapy in humans. Such studies in patients would comprise three phases. First, a Phase I safety and pharmacokinetics study would be conducted to determine the maximum tolerated dose (MTD) and to characterize the dose-limiting toxicity, pharmacokinetics and preliminary pharmacodynamics of the single agents and the combination in humans. The scheme of the phase I study would be to use single escalating doses of each of the agents measure the biochemical, PK, and clinical parameters, permitting the determination of the MTD and the threshold and maximum concentrations in dosage and in circulating drug that constitute the therapeutic window to be used in subsequent Phase II and Phase III trials, as well as defining potential toxicities and adverse events to be tracked in future studies.

Phase II clinical studies of human patients would be independently conducted in breast, endometrial, ovarian, and prostate or bladder cancer patients. Failure or lack of response to a previous anti-PD-1/anti-PD-L1 therapy would be an enrollment criteria. The trial would evaluate the efficacy and safety of the combination alone and in combination with a current chemotherapy employed in the specific indication. Patients will be administered the combination at a dose level and regimen pre-determined in Phase I with or without the standard chemotherapeutic agent. A control arm comprising of the chemotherapeutic agent plus placebo would be included. The primary endpoint would be response rate as defined by the Response Evaluation Criteria in Solid Tumors (RECIST). Secondary endpoints will include safety and tolerability, time-to-progression and overall survival.

A phase III efficacy and safety study is conducted in breast, endometrial, ovarian, and prostate or bladder cancer patients to test ability to reach statistically significant clinical endpoints such as progression-free-survival as measured by RECIST. The trial will also be statistically powered for overall survival as a secondary endpoint with projected enrollment in excess of 400 patients. Efficacy outcomes are determined using standard statistical methods. Toxicity and adverse event markers are also followed in the study to verify that the compound is safe when used in the manner described.

### Example 12: Effects of Anti-PD-L1/Anti-PD-L2 Therapy in VNMA Mice Implanted with MB49 (Bladder Carcinoma) Cells

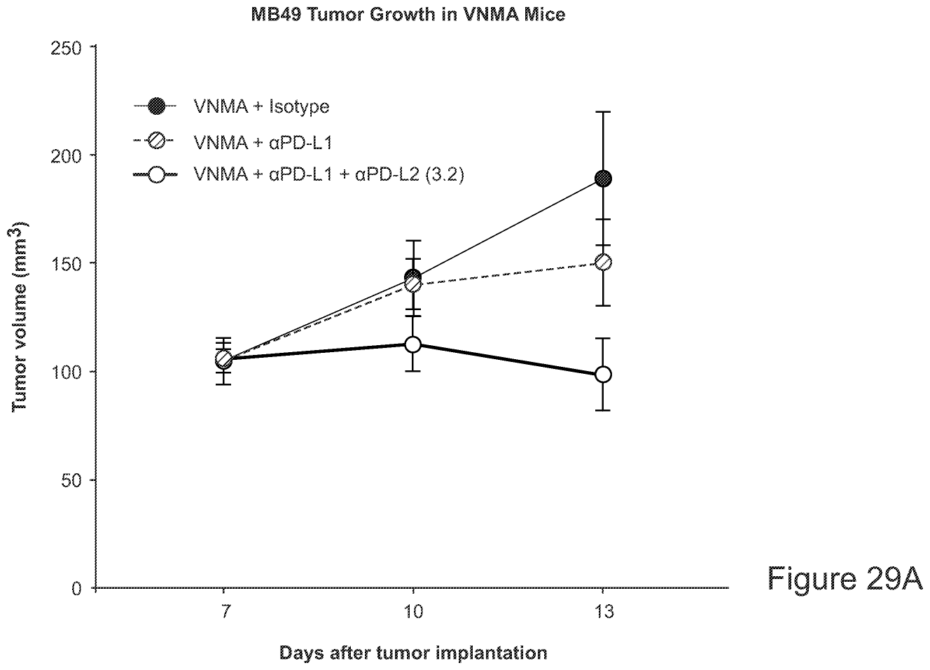

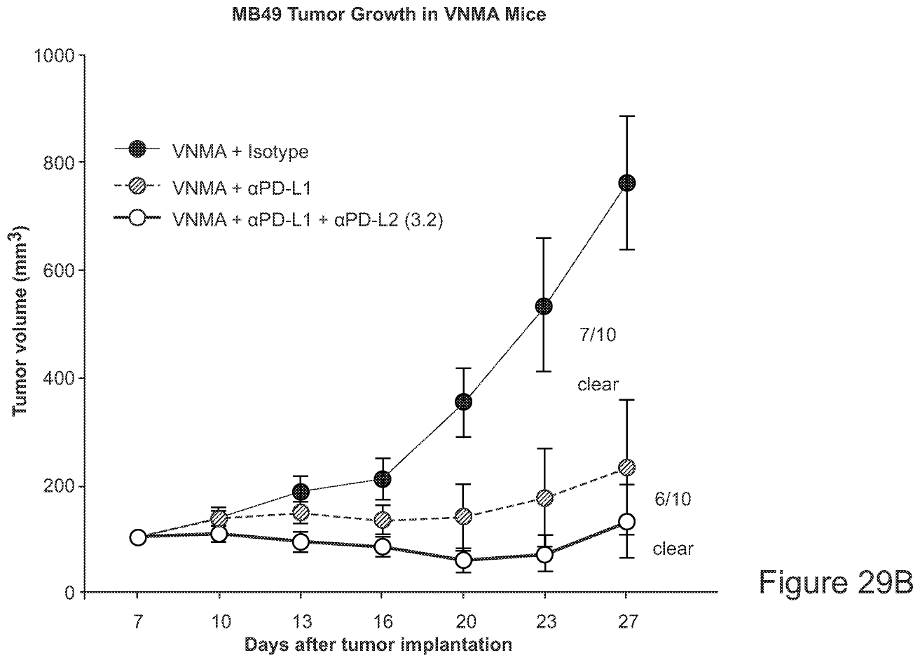

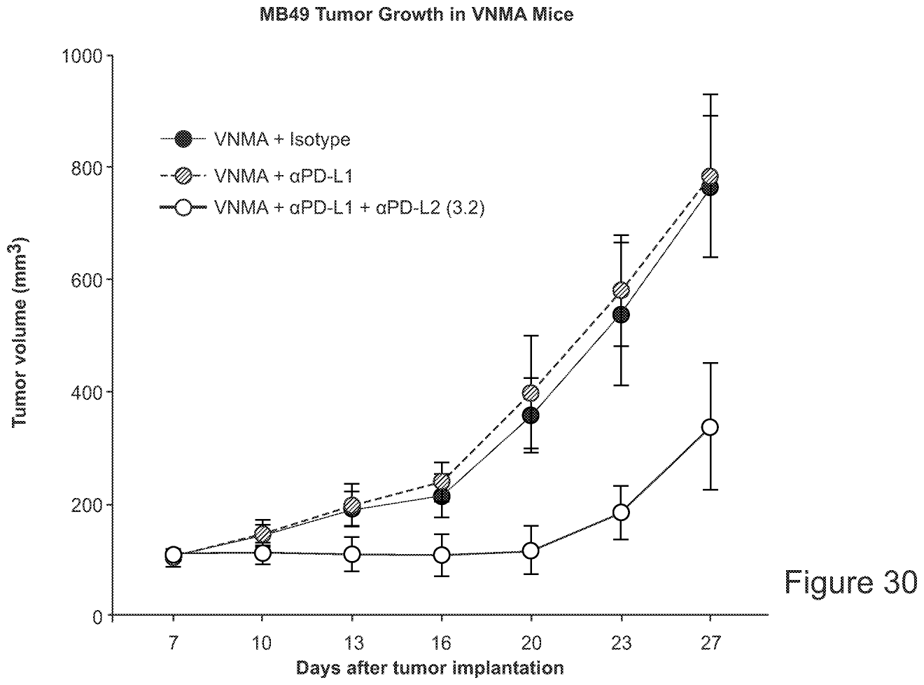

The effects of anti-PD-L1 therapy versus anti-PD-L1/anti-PD-L2 therapy on antibiotic treated mice were analyzed. Mice were given antibiotics (0.5 mg/ml Vancomycin, 1 mg/ml Neomycin, 0.25 mg/ml metronidazole benzoate, 1 mg/ml Ampicillin) in drinking water before being implanted with MB49 (bladder carcinoma) cells. On day zero, 250k of MB49 tumor cells were implanted subcutaneously on the abdomen of 6 week old female mice. On days 7, 10, 13, and 16 mice were treated with a control, an anti-PD-L1 antibody or a combination of anti-PD-L1 and anti-PD-L2 (3.2) antibodies. Tumors were measured every three to four days, and tumor volume over time produced by each treatment is depicted in FIGS. 29A-B and 30, which shows that combined anti-PD-L1 and anti-PD-L2 therapy promotes a more durable anti-tumor response in antibiotic treated mice than anti-PD-L1 therapy alone. FIG. 30 further illustrates how the PD-L1 and PD-L2 combination therapy is capable of reducing the size of tumors that continued to grow with therapy.

### Example 13: Effects of Anti-PD-1/Anti-PD-L2 Therapy in VNMA Mice Implanted with MB49 (Bladder Carcinoma) Cells

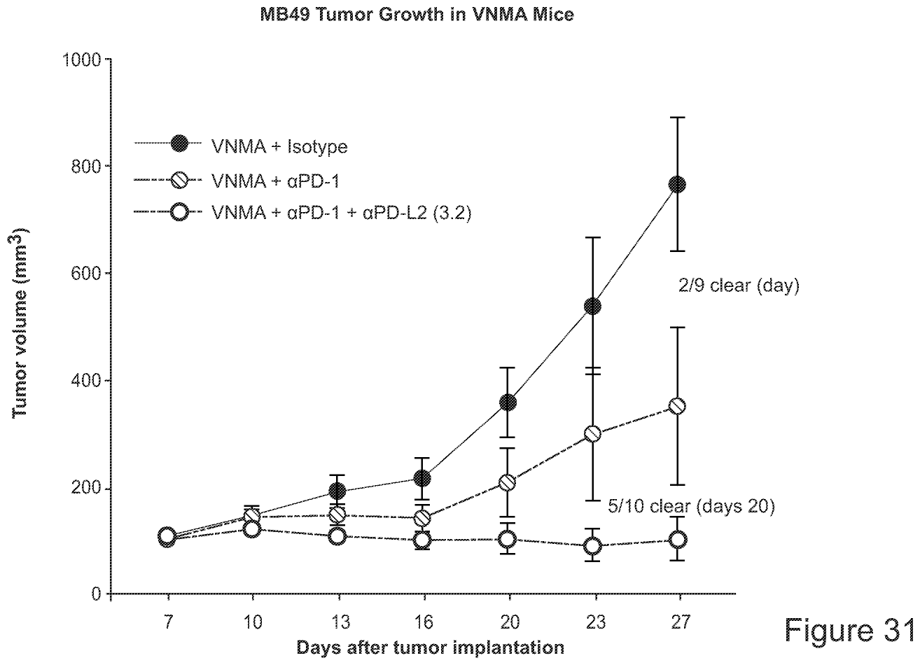

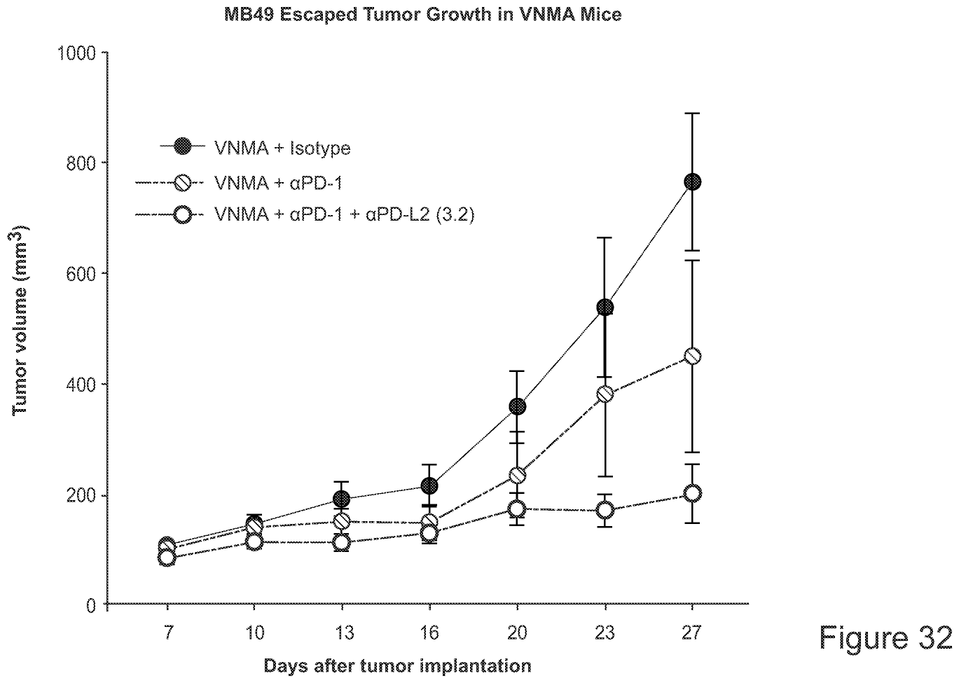

The effects of anti-PD-1 therapy versus anti-PD-1/anti-PD-L2 therapy on antibiotic treated mice were analyzed. Mice were given antibiotics (0.5 mg/ml Vancomycin, 1 mg/ml Neomycin, 0. 25 mg/ml metronidazole benzoate, 1 mg/ml Ampicillin) in drinking water before being implanted with MB49 (bladder carcinoma) cells. On day zero, 250k of MB49 tumor cells were implanted subcutaneously on the abdomen of 6 week old female mice. On days 7, 10, 13, and 16 mice were treated with a control, an anti-PD-1 antibody (RMP1-14) or a combination of anti-PD-1 and anti-PD-L2 (3.2) antibodies. Tumors were measured every three to four days, and tumor volume over time produced by each treatment is depicted in FIGS. 31 and 32, which shows that combined anti-PD-1 and anti-PD-L2 therapy promotes a more durable anti-tumor response in antibiotic treated mice than anti-PD-1 therapy alone. FIG. 30 further illustrates how the PD-1 and PD-L2 combination therapy is capable of reducing the size in tumor that continued to grow with therapy.

### Example 14: Effects of Anti-PD-1/Anti-PD-L2 Therapy in Mice Colonized with Stool from Patients with Melanoma

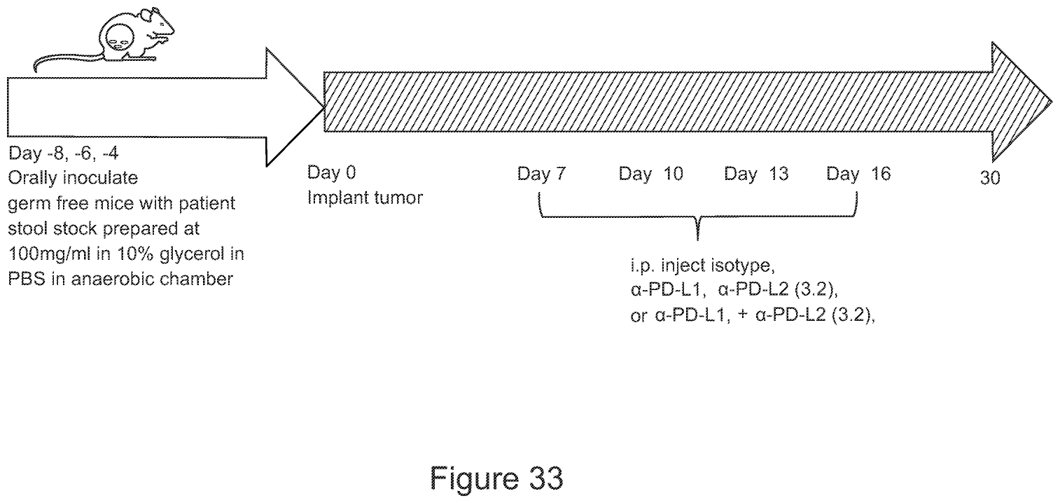

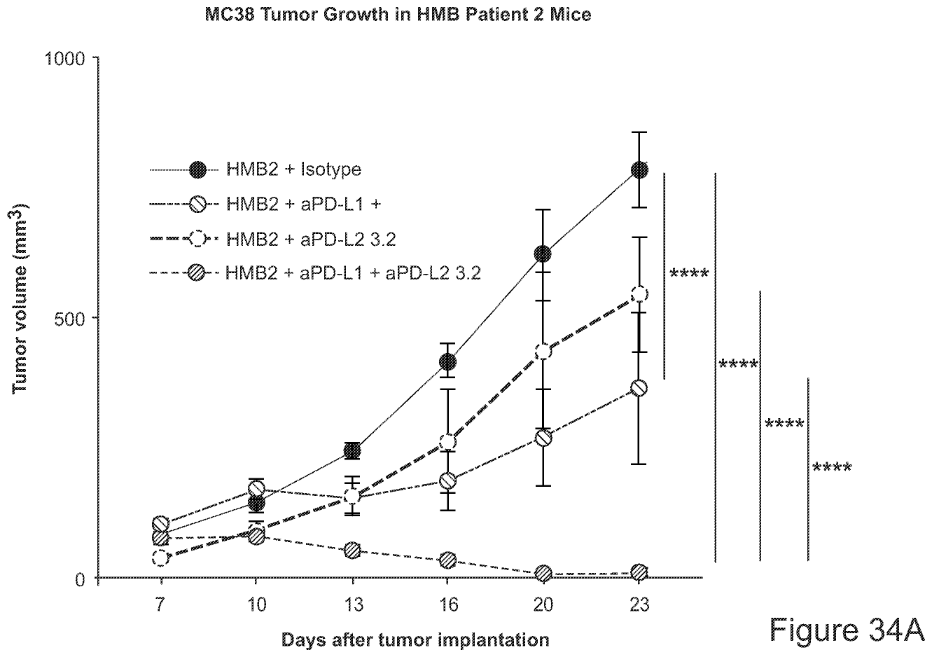

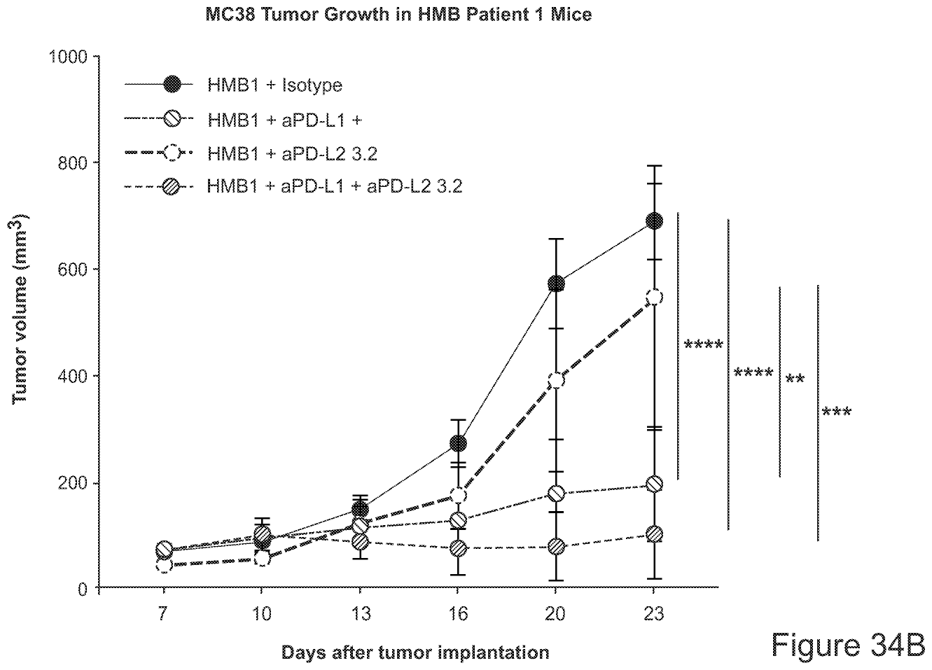

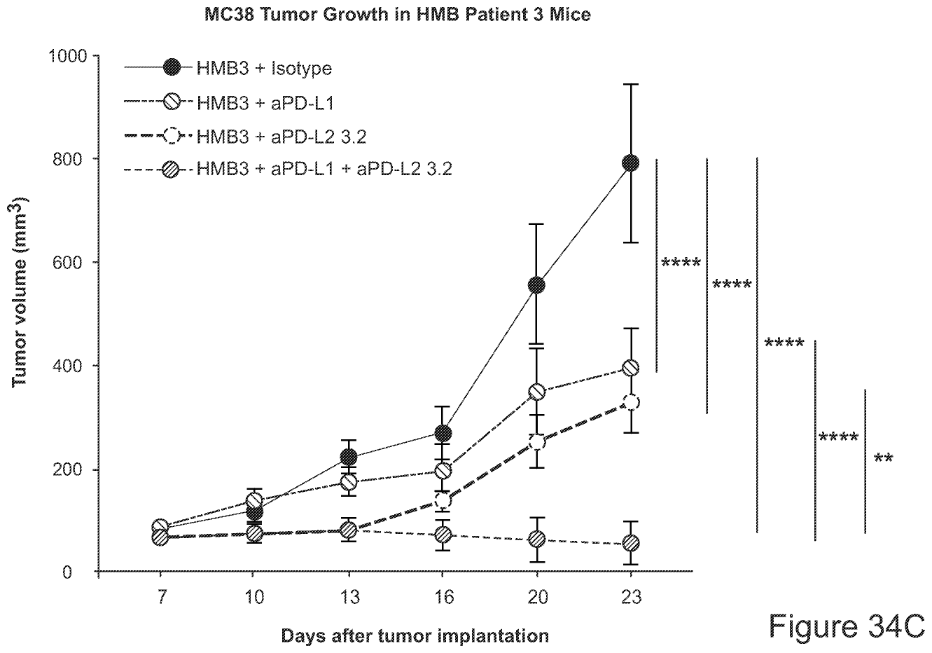

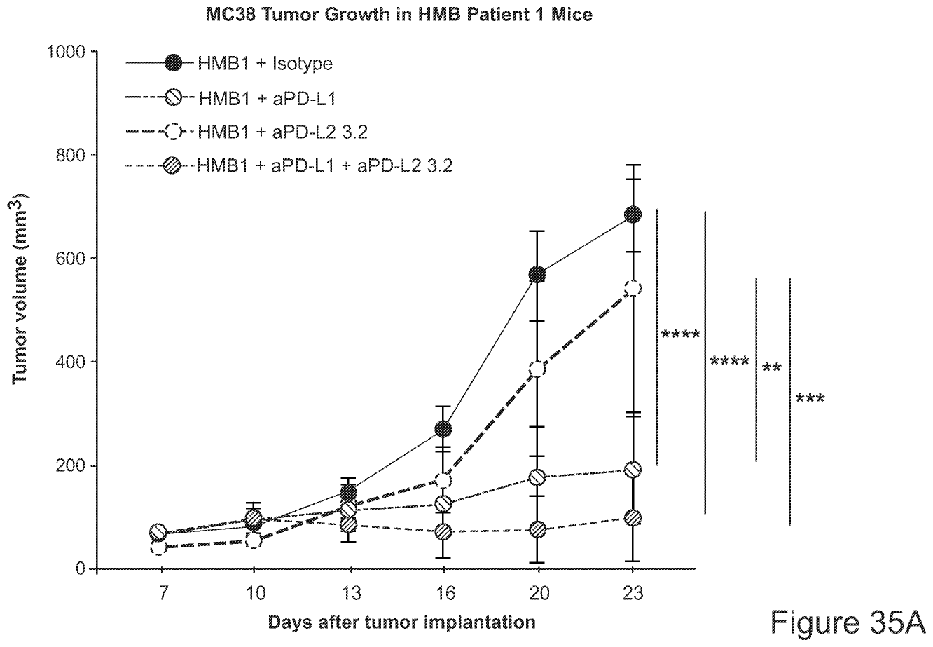

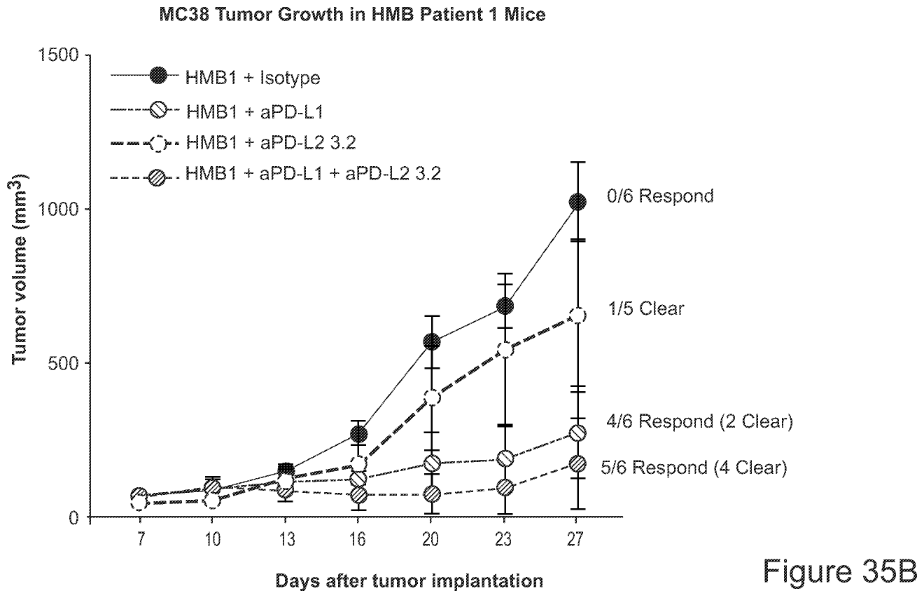

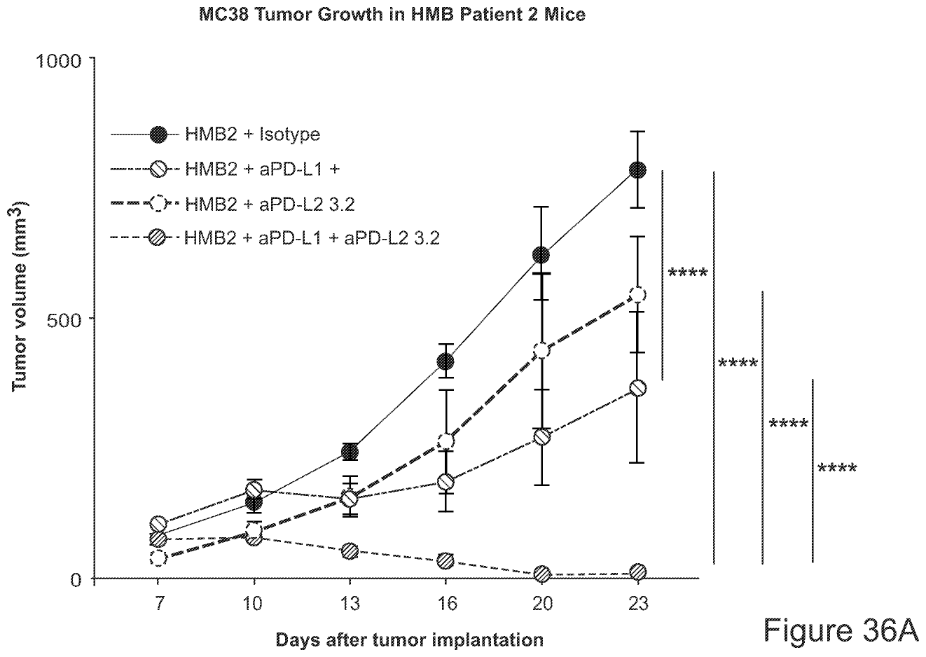

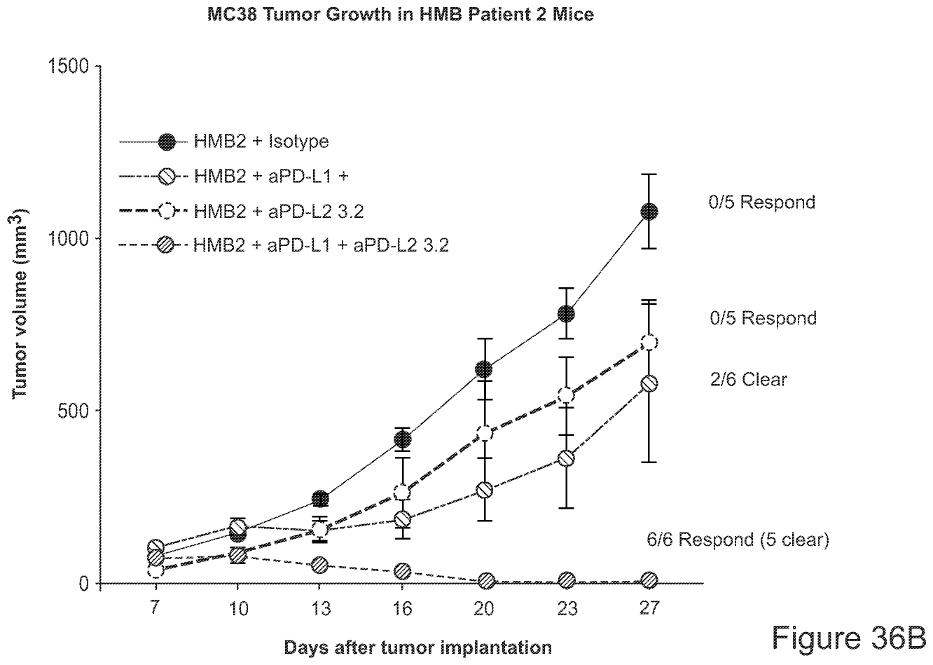

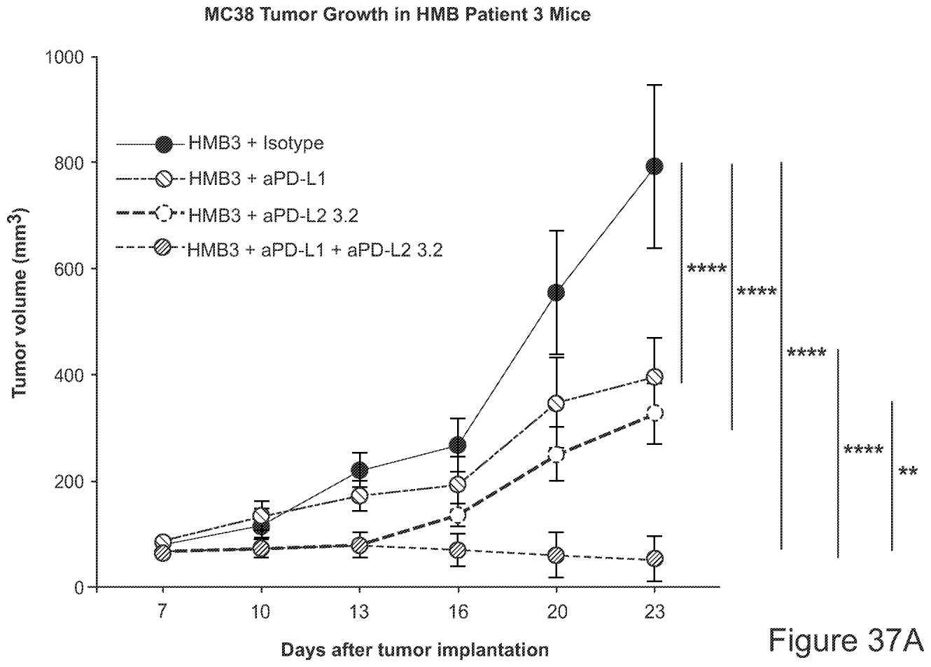

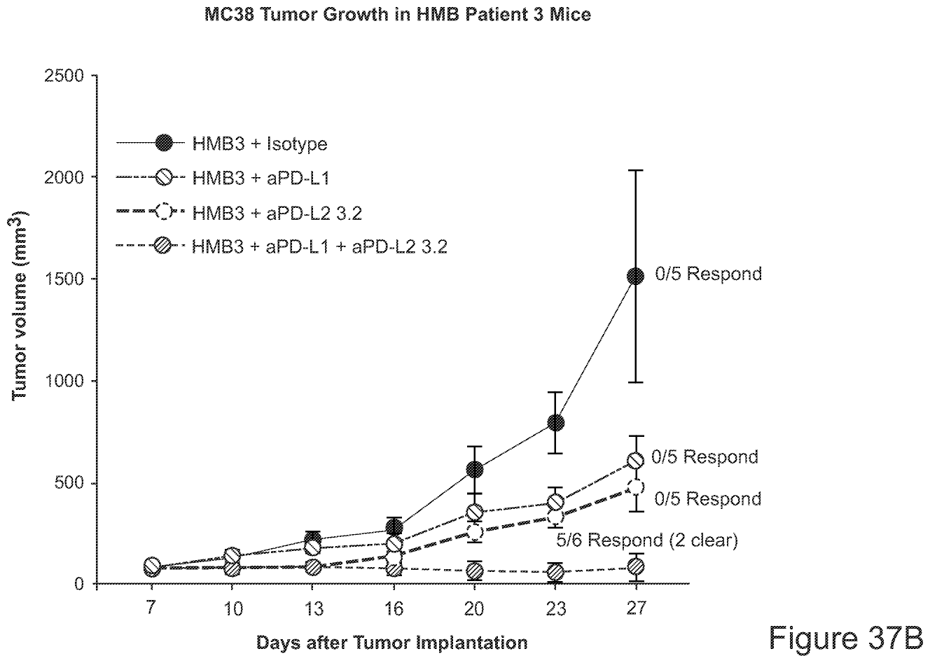

To investigate the effect of combination anti-PD-L1 plus anti-PD-L2 therapy in mice colonized with stool from patients with melanoma germ free mice were orally inoculated with stool stock from three melanoma patients. The experimental timeline is illustrated in FIG. 33. Briefly, germ-free mice were orally inoculated with patient stool stock from three patients with melanoma prepared at 100 mg/ml in 10% glycerol in PBS in an anaerobic chamber. Two of the patients were considered non-responders to anti-PD-1 therapy and one was a considered a responder to the same therapy. The specimens were blinded as to patient information. Inoculations occurred three times in dosage intervals of two days. Patient stool stock samples were obtained from Wargo/Watowich labs. Four days after the last dose of patient stool, mice were implanted with MC38 (colon adenocarcinoma) tumor cells subcutaneously. On days 7, 10, 13, and 16 mice were treated with a control, an anti-PD-L1 antibody, an anti-PD-L2 antibody or a combination of anti-PD-L1 and anti-PD-L2 (3.2) antibodies. Tumors were measured every three to five days. Tumor volume over time produced by each treatment is depicted in FIGS. 34-37. FIGS. 34A-34C depict tumor volume over time for mice treated the stool from patients 1, 2, and 3 respectively. 2 way ANOVA Tukey's multiple comparisons * P<0.05, ** P<0.01, ***P<0.001, **** P<0.00001. FIGS. 35A-B depict tumor volume over time for mice treated the stool from patient 1, a responder to PD-L1 therapy. FIGS. 36A-B depict tumor volume over time for mice treated the stool from patient 2, a non-responder to PD-L1 therapy. FIGS. 37A-B depict tumor volume over time for mice treated the stool from patient 3, a non-responder to PD-L1 therapy. FIGS. 34-37 shows that combined anti-PD-L1 and anti-PD-L2 therapy promotes a more durable anti-tumor response than anti-PD-L1 therapy alone.

### Example 15: Effects of Anti-PD-L1/Anti-PD-L2 Therapy on Patients with Melanoma

The clinical effects of an anti-PD-L2/anti-PD-L1 therapy on the patients from which the stool stock described in Example 12 were obtained was investigated. Patient 1, a responder to a PD-L1 therapy, and patients 2 and 3, non-responders to the PD-L1 therapy, were treated with a combination therapy of anti-PD-L1 and anti-PD-L2 antibodies. The combination therapy produced a significant therapeutic effect in all three patients.

### II. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various examples may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “administering” means providing a pharmaceutical agent or composition to a subject, and includes, but is not limited to, administering by a medical professional and self-administering.

As used herein, the term “about” a number refers to that number plus or minus 10% of that number. The term “about” a range refers to that range minus 10% of its lowest value and plus 10% of its greatest value.

The term “agent” is used herein to denote a chemical compound, a small molecule, a mixture of chemical compounds and/or a biological macromolecule (such as an antibody, nucleic acid, a protein, or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen-binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes, for example, monoclonal antibodies, polyclonal antibodies, chimeric antibodies, humanized antibodies, human antibodies, multi-specific antibodies (e.g., bispecific antibodies), single-chain antibodies and antigen-binding antibody fragments. An “isolated antibody,” as used herein, refers to an antibody which is substantially free of other antibodies having different antigenic specificities. An isolated antibody may, however, have some cross-reactivity to other, related antigens.

The terms “antigen-binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “PD-1” refers to a member of the immunoglobulin gene superfamily that functions as a coinhibitory receptor having PD-L1 and PD-L2 as known ligands. In some examples, PD-1 has an extracellular region containing immunoglobulin superfamily domain, a transmembrane domain, and an intracellular region including an immunoreceptor tyrosine-based inhibitory motif (ITIM).

The term “PD-L1” refers to a specific binding partner to the PD-1 receptor. Various forms of human PD-L1 molecules have been identified and are well known in the art.

The term “PD-L2” refers to another specific binding partner to the PD-1 receptor. PD-L2 is a B7 family member expressed on various antigen presenting cells, including dendritic cells, macrophages and bone-marrow derived mast cells. Various forms of human PD-L2 molecules have been identified and are well known in the art.

The term “RGMb” refers to a glycosylphophatidylinositol (GPI)-anchored member of the repulsive guidance molecule family. The nucleic acid and amino acid sequences of representative human RGMb biomarkers are well known in the art and are also available to the public at the GenBank database under NM 025239.3 and NP 079515.2. In some examples RGMb proteins are characterized by common structural elements. In some examples. RGMb proteins comprise conserved domains with homology to notch-3, phosphatidylinositol-4-phosphate-5-kinase type II beta, insulin-like growth factor binding protein-2, thrombospondin, ephrin type-B receptor 3 precursor, and Slit-2, all of which are known to influence axonal guidance, neurite outgrowth, and other neuronal developmental functions. In some examples, the C-terminus of RGMb also contains a hydrophobic domain indicative of a 21 amino acid extracellular GPI anchoring.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “humanized antibody” refers to an antibody that has at least one CDR derived from a mammal other than a human, and a FR region and the constant region of a human antibody. A humanized antibody is useful as an effective component in a therapeutic agent since antigenicity of the humanized antibody in human body is lowered.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The “tumor microenvironment” is an art-recognized term and refers to the cellular environment in which the tumor exists, and includes, for example, interstitial fluids surrounding the tumor, surrounding blood vessels, immune cells, other cells, fibroblasts, signaling molecules, and the extracellular matrix.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| # | SEQUENCE | ANNOTATION |
|---|---|---|
| 3 | gatgtgcagc ttcaggagtc gggacctggc ctggtgaaac cttctcagtc tctgtccctc | 1B9 Heavy |
| | acctgcactg tcactggcta ctcaatcacc agtgatcatg cctggaactg gatccggcag | Chain Variable |
| | gttccaggaa acaaactgga gtggatgggc tacataacct accgtggtag cactacctat | DNA |
| | agcccatctc tcaaaagtcg aatttctatc actcgagaca catccaagaa ccagttcttc | |
| | ctgcagttga attctgtgac tactgaggac acagccacat attactgtgc aagatctatg | |
| | attacgacgg ggtactatgt tatggactac tggggtcaag gaacctcagt caccgtctcc | |
| | tca | |
| 4 | gacattgtga tgacccagtc tcacaaattc atgtccacat cactaggaga cagggtcacc | 4H1 Light |
| | atcacctgca aggccagtca ggatgtgggt atttctgtag tttggtatca acagaaacca | Chain Variable |
| | gggcaatctc ctaaactact gatttactgg gcatccaccc ggcacactgg agtccctgat | DNA |
| | cgcttcacag gcagtggatc tcggacagat ttcactctga ccattaacaa tgtgcagtct | |
| | gaagacttgg cagattattt ctgtcagcaa tatagcagct atccgctcac ggtcggtgct | |
| | gggaccaagc tggagctgaa a | |
| 5 | gatgtgcagc ttcaggagtc gggacctggc ctggtgaaac cttctcagtc tctgtccctc | 4H1 Heavy |
| | acctgcactg tcactgacta ctcaatcacc agtgattatg cctggacctg gatccggcag | Chain Variable |
| | tttccgggaa acaaactgga gtggatgggc tacataacct acagaggtac cactcgctac | DNA |
| | aacccatctc tcacaagtcg aatctctttc actcgagaca catccaagaa ccagctcttc | |
| | ctgcagttga attctgtgac tactgaggac acaggcacat attgctgtgc aagatctatg | |
| | attacgacgg ggtactatgc tatggactac tggggtcaag gaacctcagt caccgtctcc | |
| | tca | |
| 6 | gacattgtga tgacccagtc tcacaaattc atgtccacat cagtaggaga cagggtcagc | 1B9 Light |
| | atctcctgca aggccagtca ggatgtgggt atttctgtag cctggtatca acagaaacca | Chain Variable |
| | gggcaatctc ctaaactact gatttactgg gcatccaccc ggcacactgg agtccctgtt | DNA |
| | cgcttcacag gcagtggatc tcggacagat ttcactctca ccataagcaa tgtgcagtct | |
| | gaagacttgg cagattattt ttgtcagcag tatagcagtt atccgcccac gttcggtgct | |
| | gggaccaagc tggagctgaa a | |
| 7 | caggtccagc tggtgcagtc tggagctgaa ctgaagaaac ctggggcctc agtgaagatg | 24F.10C12 |
| | tcctgcaagg cttctggcta cacctttact ggctacacga tgcactgggt aaaacaggcc | VH1 |
| | cctggacagg gtctggaatg gattggatac attaatccta gaagtggata tactgagtat | |
| | aatcagaagt tcaaggacag gaccacattg actgcagaca aatctaccag cacagcctac | |
| | atggaactga gcagcctgag atctgaggac tctgcggtct attattgtgc aagaccctgg | |
| | tttgcttact ggggccaagg gactctggtc actgtctctt ca | |
| 8 | caggtccagc tggtgcagtc tggagctgaa gtgaagaaac ctggggcctc | 24F.10C12 |
| | agtgaagatg tcctgcaagg cttctggcta cacctttact ggctacacga tgcactgggt | VH2 |
| | aaaacaggcc cctggacagg gtctggaatg gattggatac attaatccta gaagtggata | |
| | tactgagtat aatcagaagt tcaaggacag gaccacattg actgcagaca aatctaccag | |
| | cacagcctac atggaactga gcagcctgag atctgaggac actgcggtct attattgtgc | |
| | aagaccctgg tttgcttact ggggccaagg gactctggtc actgtctctt ca | |
| 9 | caggtccagc tggtgcagtc tggagctgaa gtgaagaaac ctggggcctc | 24F.10C12 |
| | agtgaagatg tcctgcaagg cttctggcta cacctttact ggctacacga tgcactgggt | VH3 |
| | aagacaggcc cctggacagg gtctggaatg gattggatac attaatccta gaagtggata | |
| | tactgagtat aatcagaagt tcaaggacag gaccacattg actgcagaca aatctaccag | |
| | cacagcctac atggaactga gcagcctgag atctgaggac actgcggtct attattgtgc | |
| | aagaccctgg tttgcttact ggggccaagg gactctggtc actgtctctt ca | |
| 10 | caggtccagc tggtgcagtc tggagctgaa gtgaagaaac ctggggcctc | 24F.10C12 |
| | agtgaaggtg tcctgcaagg cttctggcta cacctttact ggctacacga tgcactgggt | VH4 |
| | aagacaggcc cctggacagg gtctggaatg gattggatac attaatccta gaagtggata | |
| | tactgagtat aatcagaagt tcaaggacag gaccacattg actgcagaca aatctaccag | |
| | cacagcctac atggaactga gcagcctgag atctgaggac actgcggtct attattgtgc | |
| | aagaccctgg tttgcttact ggggccaagg gactctggtc actgtctctt ca | |
| 11 | caggtccagc tggtgcagtc tggagctgaa gtgaagaaac ctggggcctc | 24F.10C12 |
| | agtgaaggtg tcctgcaagg cttctggcta cacctttact ggctacacga tgcactgggt | VH5 |
| | aagacaggcc cctggacagg gtctggaatg gattggatac attaatccta gaagtggata | |
| | tactgagtat aatcagaagt tcaaggacag gaccacaatc actgcagaca aatctaccag | |
| | cacagcctac atggaactga gcagcctgag atctgaggac actgcggtct attattgtgc | |
| | aagaccctgg tttgcttact ggggccaagg gactctggtc actgtctctt ca | |
| 12 | gacattgtga tgacacagtc tccagcctcc ctgactgtga caccaggaga gaaggtcact | 24F.10C12 |
| | atcacctgca agtccagtca gagtctgtta aacagtggaa atcaaaagaa ctacttgacc | VL1 |
| | tggtaccagc agaaaccagg gcagcctcct aaactgttga tctactgggc atccactagg | |
| | gaatctgggg tccctgatcg cttcacaggc agtggatctg gaacagattt cactctcacc | |
| | atcagcagtc tgcaggctga agacgtggca gtttattact gtcagaatga ttatagttat | |
| | cctctcacgt tcggtcaggg gaccaagctg gagatcaaa | |
| 13 | gacattgtga tgacacagtc tccagcctcc ctgtctgtga caccaggaga gaaggtcact | 24F.10C12 |
| | atcacctgca agtccagtca gagtctgtta aacagtggaa atcaaaagaa ctacttgacc | VL2 |
| | tggtaccagc agaaaccagg gcagcctcct aaactgttga tctactgggc atccactagg | |
| | gaatctgggg tccctgatcg cttcacaggc agtggatctg gaacagattt cactctcacc | |
| | atcagcagtc tgcaggctga agacgtggca gtttattact gtcagaatga ttatagttat | |
| | cctctcacgt tcggtcaggg gaccaagctg gagatcaaa | |
| 14 | gacattgtga tgacacagtc tccagccttc ctgtctgtga caccaggaga gaaggtcact | 24F.10C12 |
| | atcacctgca agtccagtca gagtctgtta aacagtggaa atcaaaagaa ctacttgacc | VL3 |
| | tggtaccagc agaaaccagg gcagcctcct aaactgttga tctactgggc atccactagg | |
| | gaatctgggg tccctgatcg cttcacaggc agtggatctg gaacagattt cactctcacc | |
| | atcagcagtc tgcaggctga agacgtggca gtttattact gtcagaatga ttatagttat | |
| | cctctcacgt tcggtcaggg gaccaagctg gagatcaaa | |
| 15 | gacattgtga tgacacagtc tccagccttc ctgtctgtga caccaggaga gaaggtcact | 24F.10C12 |
| | atcacctgca agtccagtca gagtctgtta aacagtggaa atcaaaagaa ctacttgacc | VL4 |
| | tcgtaccagc agaaaccagg gcagcctcct aaactgttga tctactgggc atccactagg | |
| | gaatctgggg tccctgatcg cttctccggc agtggatctg gaacagattt cactctcacc | |
| | atcagcagtc tgcaggctga agacgtggca gtttattact gtcagaatga ttatagttat | |
| | cctctcacgt tcggtcaggg gaccaagctg gagatcaaa | |
| 16 | atgatggctg cagttcagct cttagggctt ttgctgctct gcctccgagc catgagatgt | 9D1 VL |
| | gacatccaga tgacccagtc tccttcacac ctgtcagcat ctgtgggaga cagagtcact | |
| | ctcagctgca aagtaagtca gaatatttac aagtacttaa actggtatca gcaaaaactt | |
| | ggagaagctc ccaaactcct gatatattat acaagctttt tgcaaacggg catcccgtca | |
| | aggttcagtg gcagtggatc tggtacagat tacacactca ccatcagcag cctgcagcct | |
| | gaagatgttg ccacatattt ctgccagaag tattatagcg ggtggacgtt cggtggaggc | |
| | accaagctgg aattgaaa | |
| 17 | atgggatgga gccagatcat tctctttctg gtggcagcaa ctacatgtgt ccactcccag | 9D1 VH |
| | gtacagctac agcaatcagg gactgaactg gtgaagcctg ggtcctcagt gaaaatttcc | |
| | tgcaaggctt ctggcgacac cttcaccagt gactatatgc actggataag gcagcagcct | |
| | ggaagtggcc ttgagtggat tgggtggatt tatcctggaa atggtaatac taagtacaat | |
| | caaaagttcg atgggaaggc aacactcact gcagacaaat cctccagcac agcctatttg | |
| | cagctcagcc tcctgacatc tgaggactat gcagtctatt tctgtgcaag acagacggag | |
| | gggtactttg attactgggg ccaaggagtc atggtcacag tctcctca | |

1. A method for treating cancer in an individual that has failed an anti-PD1/PD-L1 therapy, comprising:
a) selecting an individual that has failed a prior anti-PD1/PD-L1 therapy; and
b) administering to the individual i) a first agent that blocks or disrupts PD-L2, RGMb, or a combination thereof, and ii) a second agent that blocks or disrupts PD-L1, PD-1 or a combination thereof.

2. The method of claim 1, wherein the first agent is an antibody, a non-activating form of PD-L2 or RGMb, a fusion protein, a nucleic acid molecule that blocks PD-L2 or RGMb transcription or translation, a small molecule, or a polypeptide.

3. The method of claim 1 or claim 2, wherein the first agent is AMP-224 or CA-170.

4. The method of any of claims 1-3, wherein the first agent is an antibody.

5. The method of any of claims 1-4, wherein the first agent is an antibody that blocks or disrupts PD-L2.

6. The method of claim 5, wherein the antibody that blocks or disrupts PD-L2 is a monoclonal antibody, or an antigen binding fragment thereof.

7. The method of claim 5 or 6, wherein the antibody that blocks or disrupts PD-L2 binds the peptide sequence CFTVTVPKDLYVVEYGSN or CYRSMISYGGADYKRITV.

8. The method of claim 5, wherein the antibody that blocks or disrupts PD-L2 comprises the heavy chain variable domain amino acid sequence encoded by SEQ ID NO: 3 and the light chain variable domain amino acid sequence encoded by SEQ ID NO: 5.

9. The method of claim 5, wherein the antibody that blocks or disrupts PD-L2 comprises the heavy chain variable domain amino acid sequence encoded by SEQ ID NO: 4 and the light chain variable domain amino acid sequence encoded by SEQ ID NO: 6.

10. The method of claim 5, wherein the antibody that blocks or disrupts PD-L2 is a humanized or fully human antibody.

11. The method of any of claims 4-10, wherein the antibody that blocks or disrupts PD-L2 is a human anti-PD-L2 antibody that is structurally related to antibodies 24F.10C12, GF17.2C9, MIH37, 3.2, or TY25.

12. The method of any of claims 4-10, wherein the antibody that blocks or disrupts PD-L2 comprises a heavy chain variable region sequence comprising SEQ ID NOS:7-11 and/or a light chain variable region sequence comprising SEQ ID NO:12-14.

13. The method of any of claims 4-12, wherein the antibody that blocks or disrupts PD-L2 is a bispecific antibody.

14. The method of claim 1 or claim 2, wherein the first agent is an antibody that disrupts or blocks RGMb.

15. The method of claim 14, wherein the antibody that disrupts or blocks RGMb is a monoclonal antibody.

16. The method of claim 15, wherein the antibody that blocks or disrupts RGMb is a humanized antibody.

17. The method of any of claims 15-16, wherein the antibody that disrupts or blocks RGMb, comprises the heavy chain variable domain amino acid sequence encoded by SEQ ID NO: 17 and the light chain variable domain amino acid sequence encoded by SEQ ID NO: 16.

18. The method any of claims 15-17, wherein the antibody that blocks or disrupts RGMb is a bispecific antibody.

19. The method of any one of claims 1 to 18, wherein the second agent is an antibody.

20. The method any of claims 1 to 19, wherein the second agent is an antibody, a non-activating form of PD-L1, a fusion protein, a nucleic acid molecule that blocks PD-L1 transcription or translation, or a small molecule PD-L1 antagonist.

21. The method of any one of claims 1 to 20, wherein the second agent is an antibody that blocks PD-1.

22. The method of claim 21, wherein the antibody that blocks PD-1 is a monoclonal antibody.

23. The method of claim 21, wherein the antibody that blocks PD-1 is a humanized antibody.

24. The method of claim 21, wherein the antibody that blocks PD-1 is a bispecific antibody.

25. The method of claim 21, wherein the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188.

26. The method of any one of claims 1 to 25, wherein the second agent is an antibody that blocks PD-L1.

27. The method of claim 26, wherein the antibody that blocks PD-L1 is a monoclonal antibody.

28. The method of claim 26, wherein the antibody that blocks PD-L1 is a humanized antibody.

29. The method of claim 26, wherein the antibody that blocks PD-L1 is a bispecific antibody.

30. The method of claim 26, wherein the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

31. The method of any one of the previous claims, wherein the first agent is administered to the subject systemically.

32. The method of any one of claims 1 to 31, wherein the first agent is administered orally.

33. The method of any one of claims 1 to 31, wherein the first agent is administered parenterally.

34. The method of any one of claims 1 to 31, wherein the first agent is administered intravenously.

35. The method of any one of claims 1 to 34, wherein the second agent is administered to the subject systemically.

36. The method of any one of claims 1 to 35, wherein the second agent is administered orally.

37. The method of any one of claims 1 to 35, wherein the second agent is administered parenterally.

38. The method of any one of claims 1 to 35, wherein the second agent is administered intravenously.

39. The method of any one of claims 1-38, wherein the individual is a non-responder to an anti-PD1 or anti-PD-L1 therapy.

40. The method of any one of claims 1-38, wherein the individual is a partial responder to an anti-PD1 or anti-PD-L1 therapy.

41. The method of any one of claims 1 to 40, wherein the cancer is a head and neck cancer, a lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, a ovarian cancer, a testicular cancer, a prostate cancer, a cervical cancer, a vaginal cancer, or a bladder cancer.

42. The method of any one of claims 1-41, wherein the cancer is a bladder cancer, a colon cancer, or a melanoma.

43. The method of any one of claims 1 to 42, wherein the cancer comprises a tumor.

44. The method of claim 43, wherein the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

45. The method of claim 44, wherein the tumor is a melanoma that has been partially responsive or unresponsive to an anti-PD-L1 therapy.

46. The method of claim 44, wherein the tumor is a colorectal tumor that has been partially responsive or unresponsive to an anti-PD-L1 therapy.

47. The method of claim 44, wherein the tumor is a bladder tumor that has been partially responsive or unresponsive to an anti-PD-L1 therapy.

48. A therapeutic composition for treating an individual with cancer comprising, comprising:
a) a first agent that blocks or disrupts PD-L2, RGMb, or a combination thereof, and
b) a second agent that blocks or disrupts PD-L1, PD-1 or a combination thereof.

49. The therapeutic composition of claim 48, for use in treating an individual that has failed an anti-PD1/PD-L1 therapy.

50. The therapeutic composition of claim 48 or claim 49, wherein the first agent is an antibody, a non-activating form of PD-L2 or RGMb, a fusion protein, a nucleic acid molecule that blocks PD-L2 or RGMb transcription or translation, a small molecule, or a polypeptide.

51. The therapeutic composition of any of claims 48-50, wherein the first agent is AMP-224, CA-170, or a combination thereof.

52. The therapeutic composition of any of claims 48-50, wherein the first agent is an antibody.

53. The therapeutic composition of any of claims 48-50, wherein the first agent is an antibody that blocks or disrupts PD-L2.

54. The therapeutic composition of claim 53, wherein the antibody that blocks or disrupts PD-L2 is a monoclonal antibody, or an antigen binding fragment thereof.

55. The therapeutic composition of claim 53 or 54, wherein the antibody that blocks or disrupts PD-L2 binds the peptide sequence CFTVTVPKDLYVVEYGSN or CYRSMISYGGADYKRITV.

56. The therapeutic composition of any of claims 53-55, wherein the antibody that blocks or disrupts PD-L2 comprises the heavy chain variable domain amino acid sequence encoded by SEQ ID NO: 3 and the light chain variable domain amino acid sequence encoded by SEQ ID NO: 5.

57. The therapeutic composition of any of claims 53-56, wherein the antibody that blocks or disrupts PD-L2 comprises the heavy chain variable domain amino acid sequence encoded by SEQ ID NO: 4 and the light chain variable domain amino acid sequence encoded by SEQ ID NO: 6.

58. The therapeutic composition of any of claims 53-57, wherein the antibody that blocks or disrupts PD-L2 is a humanized antibody.

59. The therapeutic composition of any of claims 53-58, wherein the antibody that blocks or disrupts PD-L2 is a human anti-PD-L2 antibody that is structurally related to antibodies 24F.10C12, GF17.2C9, MIH37, 3.2, or TY25.

60. The therapeutic composition of any of claims 53-59, wherein the antibody that blocks or disrupts PD-L2 comprises a heavy chain variable region sequence comprising an amino acid sequence encoded by SEQ ID NOS:7-11 and/or a light chain variable region sequence comprising SEQ ID NO:12-14.

61. The therapeutic composition of any of claims 53-60, wherein the antibody that blocks or disrupts PD-L2 is a bispecific antibody.

62. The therapeutic composition of claim 48 or claim 49, wherein the first agent is an antibody that disrupts or blocks RGMb.

63. The therapeutic composition of claim 62, wherein the antibody that disrupts or blocks RGMb is a monoclonal antibody.

64. The therapeutic composition of any of claims 62-63, wherein the antibody that blocks or disrupts RGMb is a humanized antibody.

65. The therapeutic composition of any of claims 62-63, wherein the antibody that disrupts or blocks RGMb, comprises the heavy chain variable domain amino acid sequence encoded by SEQ ID NO: 17 and the light chain variable domain amino acid sequence encoded by SEQ ID NO: 16.

66. The therapeutic composition of any of claims 62-63, wherein the antibody that blocks or disrupts RGMb is a bispecific antibody.

67. The therapeutic composition of any one of claims 62-66, wherein the second agent is an antibody.

68. The therapeutic composition of any one of claims 48 to 67, wherein the second agent is an antibody, a non-activating form of PD-L1, a nucleic acid molecule that blocks PD-L1 transcription or translation, or a small molecule PD-L1 antagonist.

69. The therapeutic composition of any one of claims 48 to 67, wherein the second agent is an antibody that blocks PD-1.

70. The therapeutic composition of claim 69, wherein the antibody that blocks PD-1 is a monoclonal antibody.

71. The therapeutic composition of claim 69, wherein the antibody that blocks PD-1 is a humanized antibody.

72. The therapeutic composition of claim 69, wherein the antibody that blocks PD-1 is a bispecific antibody.

73. The therapeutic composition of claim 69, wherein the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188.

74. The therapeutic composition of any one of claims 48 to 67, wherein the second agent is an antibody that blocks PD-L1.

75. The therapeutic composition of claim 74, wherein the antibody that blocks PD-L1 is a monoclonal antibody.

76. The therapeutic composition of claim 74, wherein the antibody that blocks PD-L1 is a humanized antibody.

77. The therapeutic composition of claim 74, wherein the antibody that blocks PD-L1 is a bispecific antibody.

78. The therapeutic composition of claim 74, wherein the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

79. The therapeutic composition of any one of claims 48-78, wherein the composition is administered to the subject systemically.

80. The therapeutic composition of any one of claims 48-78, wherein the composition is administered orally.

81. The therapeutic composition of any one of claims 48-78, wherein the composition is administered parenterally.

82. The therapeutic composition of any one of claims 48-78, wherein the composition is administered intravenously.

83. The therapeutic composition of any of claims 48 to 82, wherein the cancer is a head and neck cancer lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, a ovarian cancer, a testicular cancer, a prostate cancer, a cervical cancer, a vaginal cancer, or a bladder cancer.

84. The therapeutic composition any one of claims 48 to 82, wherein the cancer comprises a tumor.

85. The therapeutic composition of claim 84, wherein the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

86. A kit comprising:
a) a first agent that blocks or disrupts PD-L2, RGMb, or a combination thereof;
b) a second agent that disrupts PD-L1, PD-1 or a combination thereof; and
c) instructions for use of the first agent and the second agent in treating a cancer in an individual.

87. The kit of claim 86, wherein the first agent is an antibody, a non-activating form of PD-L2 or RGMb, a fusion protein, a nucleic acid molecule that blocks PD-L2 or RGMb transcription or translation, a small molecule, or a polypeptide.

88. The kit of any claim 86 or claim 87, wherein the first agent is AMP-224, CA-170, or a combination thereof.

89. The kit of any of claims 86-88, wherein the first agent is an antibody.

90. The kit of any of claims 86-89, wherein the first agent is an antibody that blocks or disrupts PD-L2.

91. The kit of claim 90, wherein the antibody that blocks or disrupts PD-L2 is a monoclonal antibody, or an antigen binding fragment thereof.

92. The kit of claim 90, wherein the antibody that blocks or disrupts PD-L2 binds the peptide sequence CFTVTVPKDLYVVEYGSN or CYRSMISYGGADYKRITV.

93. The kit of any of claims 91-92, wherein the antibody that blocks or disrupts PD-L2 comprises the heavy chain variable domain amino acid sequence encoded by SEQ ID NO: 3 and the light chain variable domain amino acid sequence encoded by SEQ ID NO: 5.

94. The kit of any of claims 91-92, wherein the antibody that blocks or disrupts PD-L2 comprises the heavy chain variable domain amino acid sequence encoded by SEQ ID NO: 4 and the light chain variable domain amino acid sequence encoded by SEQ ID NO: 6.

95. The kit of any of claims 91-92, wherein the antibody that blocks or disrupts PD-L2 is a humanized antibody.

96. The kit of any of claims 91-92, wherein the antibody that blocks or disrupts PD-L2 is a human anti-PD-L2 antibody that is structurally related to antibodies 24F.10C12, GF17.2C9, MIH37, 3.2, or TY25.

97. The kit of any of claims 91-92, wherein the antibody that blocks or disrupts PD-L2 comprises a heavy chain variable region sequence comprising SEQ ID NOS:7-11 and/or a light chain variable region sequence comprising SEQ ID NOS:12-15.

98. The kit of any of claims 91-92, wherein the antibody that blocks or disrupts PD-L2 comprises a heavy chain variable region sequence of SEQ ID NO: 13 or 14, and a light chain variable region sequence of SEQ ID NO: 15, 16, or 17.

99. The kit of any of claims 91-92, wherein the antibody that blocks or disrupts PD-L2 is a bispecific antibody.

100. The kit of claim 86 or 87, wherein the first agent is an antibody that disrupts or blocks RGMb.

101. The kit of claim 100, wherein the antibody that disrupts or blocks RGMb is a monoclonal antibody.

102. The kit of claim 101, wherein the antibody that blocks or disrupts RGMb is a humanized antibody.

103. The kit of any of claims 100-102, wherein the antibody that disrupts or blocks RGMb is a human anti-RGMb antibody that is structurally related to 307.9D1, 307.8B2, 307.1H6, 307.9D3, or 307.5G1.

104. The kit of any of claims 100-103, wherein the antibody that disrupts or blocks RGMb, comprises the heavy chain variable domain amino acid sequence encoded by SEQ ID NO: 17 and the light chain variable domain amino acid sequence encoded by SEQ ID NO: 16.

105. The kit of any of claims 100-104, wherein the antibody that disrupts or blocks RGMb, wherein the antibody that blocks or disrupts RGMb is a bispecific antibody.

106. The kit of any one of claims 100-105, wherein the second agent is an antibody, a non-activating form of PD-L1, a fusion protein, a nucleic acid molecule that blocks PD-L1 or transcription or translation, a small molecule, or a polypeptide.

107. The kit of any one of claims 86-105, wherein the second agent is an antibody that blocks PD-1.

108. The kit of claim 107, wherein the antibody that blocks PD-1 is a monoclonal antibody.

109. The kit of claim 107, wherein the antibody that blocks PD-1 is a humanized antibody.

110. The kit of claim 107, wherein the antibody that blocks PD-1 is a bispecific antibody.

111. The kit of claim 107, wherein the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188.

112. The kit of any one of claims 86-105, wherein the second agent is an antibody that blocks PD-L1.

113. The kit of claim 112, wherein the antibody that blocks PD-L1 is a monoclonal antibody.

114. The kit of claim 112, wherein the antibody that blocks PD-L1 is a humanized antibody.

115. The kit of claim 112, wherein the antibody that blocks PD-L1 is a bispecific antibody.

116. The kit of claim 112, wherein the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.

117. The kit of any one of the previous claims, wherein the first agent is administered to the subject systemically.

118. The kit of any of claims 86-117, wherein the first agent is administered orally.

119. The kit of any of claims 86-117, wherein the first agent is administered parenterally.

120. The kit of any of claims 86-117, wherein the first agent is administered intravenously.

121. The kit of any of claims 86-120, wherein the second agent is administered to the subject systemically.

122. The kit of any of claims 86-120, wherein the second agent is administered orally.

123. The kit of any of claims 86-120, wherein the second agent is administered parenterally.

124. The kit of any of claims 86-120, wherein the second agent is administered intravenously.

125. The kit of any of claims 86-124, wherein the cancer is a head and neck cancer lung cancer, a breast cancer, a colon cancer, a cervical cancer, a pancreatic cancer, a renal cancer, a stomach cancer, a GI cancer, a liver cancer, a bone cancer, a hematological cancer, a neural tissue cancer, a melanoma, a thyroid cancer, a ovarian cancer, a testicular cancer, a prostate cancer, a cervical cancer, a vaginal cancer, or a bladder cancer.

126. The kit of any of claims 86-125, wherein the cancer comprises a tumor.

127. The kit of any of claims 86-125, wherein the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

128. A method for treating cancer in an individual that has failed a therapy selected from an anti-PD1 therapy or an anti-PD-L1 therapy, comprising administering to the individual i) a first agent that blocks or disrupts PD-L2, RGMb, or a combination thereof, and ii) a second agent that blocks or disrupts PD-L1, PD-1 or a combination thereof.

129. The method of claim 128, wherein the cancer is refractory to an anti-PD1 therapy or an anti-PD-L1 therapy.

130. The method of claim 128, wherein the cancer is not responsive to an anti-PD1 therapy or an anti-PD-L1 therapy.

131. The method of claim 128, wherein the cancer has relapsed following an anti-PD1 therapy or an anti-PD-L1 therapy.

132. The method of any of claims 128-131, wherein the patient has only been treated with the anti-PD1 therapy.

133. The method of any of claims 129-131, wherein the patient has only been treated with the anti-PD-L1 therapy.

134. The method of any of claim 128-131, wherein the patient has been treated with both the anti-PD1 therapy and the anti-PD-L1 therapy.

135. The method of any of claims 128-131, wherein the anti-PD1 therapy is an antibody therapy.

136. The method of any of claims 128-131, wherein the anti-PD-L1 therapy is selected from an antibody, a non-activating form of PD-L1, a fusion protein, a nucleic acid molecule that blocks PD-L1 transcription or translation, or a small molecule PD-L1 antagonist.

137. The method of claim 136, wherein the anti-PD-L1 therapy is an antibody therapy.

138. The method of any of claims 128-137, wherein the first agent is an antibody, a non-activating form of PD-L2 or RGMb, a fusion protein, a nucleic acid molecule that blocks PD-L2 or RGMb transcription or translation, a small molecule, or a polypeptide.

139. The method of any of claims 128-138, wherein the first agent is AMP-224 or CA-170.

140. The method of any of claims 128-139, wherein the first agent is an antibody.

141. The method of any of claims 128-140, wherein the first agent is an antibody that blocks or disrupts PD-L2.

142. The method of claim 141, wherein the antibody that blocks or disrupts PD-L2 is a monoclonal antibody, or an antigen binding fragment thereof.

143. The method of claim 141 or 142, wherein the antibody that blocks or disrupts PD-L2 binds the peptide sequence CFTVTVPKDLYVVEYGSN or CYRSMISYGGADYKRITV.

144. The method of claim 141, wherein the antibody that blocks or disrupts PD-L2 comprises the heavy chain variable domain amino acid sequence encoded by SEQ ID NO: 3 and the light chain variable domain amino acid sequence encoded by SEQ ID NO: 5.

145. The method of claim 141, wherein the antibody that blocks or disrupts PD-L2 comprises the heavy chain variable domain amino acid sequence encoded by SEQ ID NO: 4 and the light chain variable domain amino acid sequence encoded by SEQ ID NO: 6.

146. The method of claim 141, wherein the antibody that blocks or disrupts PD-L2 is a humanized or fully human antibody.

147. The method of any of claims 128-146, wherein the antibody that blocks or disrupts PD-L2 is a human anti-PD-L2 antibody that is structurally related antibodies 24F.10C12, GF17.2C9, MIH37, 3.2, or TY25.

148. The method of any of claims 128-147, wherein the antibody that blocks or disrupts PD-L2 comprises a heavy chain variable region sequence comprising SEQ ID NOS:7-11 and/or a light chain variable region sequence comprising SEQ ID NO:12-14.

149. The method of any of claims 128-148, wherein the antibody that blocks or disrupts PD-L2 is a bispecific antibody.

150. The method of claim 128, wherein the first agent is an antibody that disrupts or blocks RGMb.

151. The method of claim 150, wherein the antibody that disrupts or blocks RGMb is a monoclonal antibody.

152. The method of claim 150 or claim 151, wherein the antibody that blocks or disrupts RGMb is a humanized antibody.

153. The method of any of claims 150-152, wherein the antibody that disrupts or blocks RGMb comprises the heavy chain variable domain amino acid sequence encoded by SEQ ID NO: 17 and the light chain variable domain amino acid sequence encoded by SEQ ID NO: 16.

154. The method any of claims 150-153, wherein the antibody that blocks or disrupts RGMb is a bispecific antibody.

155. The method of any one of claim 154, wherein the second agent is an antibody.

156. The method any of claims 128 to 155, wherein the second agent is an antibody, a non-activating form of PD-L1, a fusion protein, a nucleic acid molecule that blocks PD-L1 transcription or translation, or a small molecule PD-L1 antagonist.

157. The method of any one of claims 128-156, wherein the second agent is an antibody that blocks PD-1.

158. The method of claim 157, wherein the antibody that blocks PD-1 is a monoclonal antibody.

159. The method of claim 157, wherein the antibody that blocks PD-1 is a humanized antibody.

160. The method of claim 157, wherein the antibody that blocks PD-1 is a bispecific antibody.

161. The method of claim 157, wherein the antibody that blocks PD-1 is selected from cemiplimab (REGN2810), nivolumab (BMS-936558, MDX-1106, ONO-4538), pembrolizumab (MK-3475, SCH 900475), SHR1210, sintilimab (IBI308), spartalizumab (PDR001), tislelizumab (BGB-A317), pidilizumab, BCD-100, toripalimab (JS001), PF-06801591, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, and CX-188.

162. The method of any one of claims 128 to 156, wherein the second agent is an antibody that blocks PD-L1.

163. The method of claim 162, wherein the antibody that blocks PD-L1 is a monoclonal antibody.

164. The method of claim 162, wherein the antibody that blocks PD-L1 is a humanized antibody.

165. The method of claim 162, wherein the antibody that blocks PD-L1 is a bispecific antibody.

166. The method of claim 162, wherein the antibody that blocks PD-L1 is selected from atezolizumab (MPDL3280A, RG7446, R05541267), durvalumab (MEDI4736, MEDI-4736), avelumab (MSB0010718C), FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, and LY3415244.


SEQUENCE LISTING (17 sequences)

SEQ ID NO: 1 - protein, 18 aa, Unknown
  source: /note=“Description of Unknown PD-L2 sequence”
CFTVTVPKDLYVVEYGSN

SEQ ID NO: 2 - protein, 18 aa, Unknown
  source: /note=“Description of Unknown PD-L2 sequence”
CYRSMISYGGADYKRITV

SEQ ID NO: 3 - DNA, 363 bp, Artificial Sequence
  source: /note=“Description of Artificial Sequence Synthetic polynucleotide”
gatgtgcagcttcaggagtcgggacctggcctggtgaaaccttctcagtctctgtccctcacctgcactgtcactggctactcaatcaccagtgatcatgcctggaactggatccggcaggttccaggaaacaaactggagtggatgggctacataacctaccgtggtagcactacctatagcccatctctcaaaagtcgaatttctatcactcgagacacatccaagaaccagttcttcctgcagttgaattctgtgactactgaggacacagccacatattactgtgcaagatctatgattacgacggggtactatgttatggactactggggtcaaggaacctcagtcaccgtctcctca

SEQ ID NO: 4 - DNA, 321 bp, Artificial Sequence
  source: /note=“Description of Artificial Sequence Synthetic polynucleotide”
gacattgtgatgacccagtctcacaaattcatgtccacatcactaggagacagggtcaccatcacctgcaaggccagtcaggatgtgggtatttctgtagtttggtatcaacagaaaccagggcaatctcctaaactactgatttactgggcatccacccggcacactggagtccctgatcgcttcacaggcagtggatctgggacagatttcactctgaccattaacaatgtgcagtctgaagacttggcagattatttctgtcagcaatatagcagctatccgctcacggtcggtgctgggaccaagctggagctgaaa

SEQ ID NO: 5 - DNA, 363 bp, Artificial Sequence
  source: /note=“Description of Artificial Sequence Synthetic polynucleotide”
gatgtgcagcttcaggagtcgggacctggcctggtgaaaccttctcagtctctgtccctcacctgcactgtcactgactactcaatcaccagtgattatgcctggacctggatccggcagtttccgggaaacaaactggagtggatgggctacataacctacagaggtaccactcgctacaacccatctctcacaagtcgaatctctttcactcgagacacatccaagaaccagctcttcctgcagttgaattctgtgactactgaggacacaggcacatattgctgtgcaagatctatgattacgacggggtactatgctatggactactggggtcaaggaacctcagtcaccgtctcctca

SEQ ID NO: 6 - DNA, 321 bp, Artificial Sequence
  source: /note=“Description of Artificial Sequence Synthetic polynucleotide”
gacattgtgatgacccagtctcacaaattcatgtccacatcagtaggagacagggtcagcatctcctgcaaggccagtcaggatgtgggtatttctgtagcctggtatcaacagaaaccagggcaatctcctaaactactgatttactgggcatccacccggcacactggagtccctgttcgcttcacaggcagtggatctgggacagatttcactctcaccataagcaatgtgcagtctgaagacttggcagattatttttgtcagcagtatagcagttatccgcccacgttcggtgctgggaccaagctggagctgaaa

SEQ ID NO: 7 - DNA, 342 bp, Artificial Sequence
  source: /note=“Description of Artificial Sequence Synthetic polynucleotide”
caggtccagctggtgcagtctggagctgaactgaagaaacctggggcctcagtgaagatgtcctgcaaggcttctggctacacctttactggctacacgatgcactgggtaaaacaggcccctggacagggtctggaatggattggatacattaatcctagaagtggatatactgagtataatcagaagttcaaggacaggaccacattgactgcagacaaatctaccagcacagcctacatggaactgagcagcctgagatctgaggactctgcggtctattattgtgcaagaccctggtttgcttactggggccaagggactctggtcactgtctcttca

SEQ ID NO: 8 - DNA, 342 bp, Artificial Sequence
  source: /note=“Description of Artificial Sequence Synthetic polynucleotide”
caggtccagctggtgcagtctggagctgaagtgaagaaacctggggcctcagtgaagatgtcctgcaaggcttctggctacacctttactggctacacgatgcactgggtaaaacaggcccctggacagggtctggaatggattggatacattaatcctagaagtggatatactgagtataatcagaagttcaaggacaggaccacattgactgcagacaaatctaccagcacagcctacatggaactgagcagcctgagatctgaggacactgcggtctattattgtgcaagaccctggtttgcttactggggccaagggactctggtcactgtctcttca

SEQ ID NO: 9 - DNA, 342 bp, Artificial Sequence
  source: /note=“Description of Artificial Sequence Synthetic polynucleotide”
caggtccagctggtgcagtctggagctgaagtgaagaaacctggggcctcagtgaagatgtcctgcaaggcttctggctacacctttactggctacacgatgcactgggtaagacaggcccctggacagggtctggaatggattggatacattaatcctagaagtggatatactgagtataatcagaagttcaaggacaggaccacattgactgcagacaaatctaccagcacagcctacatggaactgagcagcctgagatctgaggacactgcggtctattattgtgcaagaccctggtttgcttactggggccaagggactctggtcactgtctcttca

SEQ ID NO: 10 - DNA, 342 bp, Artificial Sequence
  source: /note=“Description of Artificial Sequence Synthetic polynucleotide”
caggtccagctggtgcagtctggagctgaagtgaagaaacctggggcctcagtgaaggtgtcctgcaaggcttctggctacacctttactggctacacgatgcactgggtaagacaggcccctggacagggtctggaatggattggatacattaatcctagaagtggatatactgagtataatcagaagttcaaggacaggaccacattgactgcagacaaatctaccagcacagcctacatggaactgagcagcctgagatctgaggacactgcggtctattattgtgcaagaccctggtttgcttactggggccaagggactctggtcactgtctcttca

SEQ ID NO: 11 - DNA, 342 bp, Artificial Sequence
  source: /note=“Description of Artificial Sequence Synthetic polynucleotide”
caggtccagctggtgcagtctggagctgaagtgaagaaacctggggcctcagtgaaggtgtcctgcaaggcttctggctacacctttactggctacacgatgcactgggtaagacaggcccctggacagggtctggaatggattggatacattaatcctagaagtggatatactgagtataatcagaagttcaaggacaggaccacaatcactgcagacaaatctaccagcacagcctacatggaactgagcagcctgagatctgaggacactgcggtctattattgtgcaagaccctggtttgcttactggggccaagggactctggtcactgtctcttca

SEQ ID NO: 12 - DNA, 339 bp, Artificial Sequence
  source: /note=“Description of Artificial Sequence Synthetic polynucleotide”
gacattgtgatgacacagtctccagcctccctgactgtgacaccaggagagaaggtcactatcacctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtctgcaggctgaagacgtggcagtttattactgtcagaatgattatagttatcctctcacgttcggtcaggggaccaagctggagatcaaa

SEQ ID NO: 13 - DNA, 339 bp, Artificial Sequence
  source: /note=“Description of Artificial Sequence Synthetic polynucleotide”
gacattgtgatgacacagtctccagcctccctgtctgtgacaccaggagagaaggtcactatcacctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtctgcaggctgaagacgtggcagtttattactgtcagaatgattatagttatcctctcacgttcggtcaggggaccaagctggagatcaaa

SEQ ID NO: 14 - DNA, 339 bp, Artificial Sequence
  source: /note=“Description of Artificial Sequence Synthetic polynucleotide”
gacattgtgatgacacagtctccagccttcctgtctgtgacaccaggagagaaggtcactatcacctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtctgcaggctgaagacgtggcagtttattactgtcagaatgattatagttatcctctcacgttcggtcaggggaccaagctggagatcaaa

SEQ ID NO: 15 - DNA, 339 bp, Artificial Sequence
  source: /note=“Description of Artificial Sequence Synthetic polynucleotide”
gacattgtgatgacacagtctccagccttcctgtctgtgacaccaggagagaaggtcactatcacctgcaagtccagtcagagtctgttaaacagtggaaatcaaaagaactacttgacctggtaccagcagaaaccagggcagcctcctaaactgttgatctactgggcatccactagggaatctggggtccctgatcgcttctccggcagtggatctggaacagatttcactctcaccatcagcagtctgcaggctgaagacgtggcagtttattactgtcagaatgattatagttatcctctcacgttcggtcaggggaccaagctggagatcaaa

SEQ ID NO: 16 - DNA, 378 bp, Artificial Sequence
  source: /note=“Description of Artificial Sequence Synthetic polynucleotide”
atgatggctgcagttcagctcttagggcttttgctgctctgcctccgagccatgagatgtgacatccagatgacccagtctccttcacacctgtcagcatctgtgggagacagagtcactctcagctgcaaagtaagtcagaatatttacaagtacttaaactggtatcagcaaaaacttggagaagctcccaaactcctgatatattatacaagctttttgcaaacgggcatcccgtcaaggttcagtggcagtggatctggtacagattacacactcaccatcagcagcctgcagcctgaagatgttgccacatatttctgccagaagtattatagcgggtggacgttcggtggaggcaccaagctggaattgaaa

SEQ ID NO: 17 - DNA, 408 bp, Artificial Sequence
  source: /note=“Description of Artificial Sequence Synthetic polynucleotide”
atgggatggagccagatcattctctttctggtggcagcaactacatgtgtccactcccaggtacagctacagcaatcagggactgaactggtgaagcctgggtcctcagtgaaaatttcctgcaaggcttctggcgacaccttcaccagtgactatatgcactggataaggcagcagcctggaagtggccttgagtggattgggtggatttatcctggaaatggtaatactaagtacaatcaaaagttcgatgggaaggcaacactcactgcagacaaatcctccagcacagcctatttgcagctcagcctcctgacatctgaggactatgcagtctatttctgtgcaagacagacggaggggtactttgattactggggccaaggagtcatggtcacagtctcctca